(19) United States
(12) Patent Application Publication
(10) Pub. No.: US 2025/0108017 A1
(43) Pub. Date: Apr. 3, 2025

(54) ENHANCED DELIVERY EPINEPHRINE AND PRODRUG COMPOSITIONS

(71) Applicant: Aquestive Therapeutics, Inc., Warren, NJ (US)

(72) Inventors: Alexander Mark Schobel, Vero Beach, FL (US); Stephanie Marie Varjan, Oradell, NJ (US); Stephen Paul Wargacki, Pittstown, NJ (US)

(73) Assignee: Aquestive Therapeutics, Inc., Warren, NJ (US)

(21) Appl. No.: 18/778,562

(22) Filed: Jul. 19, 2024

Related U.S. Application Data
(60) Provisional application No. 63/514,743, filed on Jul. 20, 2023.
(60) Provisional application No. 63/564,767, filed on Mar. 13, 2024.

(51) Int. Cl. A61K 31/137; A61K 9/00; A61K 31/015; A61K 31/085; A61K 31/201; A61K 31/235; A61K 31/25; A61K 31/36; A61K 36/61; A61K 47/10; A61K 47/38

(52) U.S. Cl. CPC A61K 31/137; A61K 9/006; A61K 31/015; A61K 31/085; A61K 31/201; A61K 31/235; A61K 31/25; A61K 31/36; A61K 36/61; A61K 47/10; A61K 47/38

(57) ABSTRACT

Pharmaceutical compositions of epinephrine and its prodrugs are described and the compositions having enhanced active component permeation properties are described as being administered with specific residence times.

### CLAIM OF PRIORITY

This application claims priority to U.S. Provisional Application No. 63/514,743, filed July 2023, and U.S. Provisional Patent Application No. 63/564,767, filed Mar. 13, 2024, each of which is incorporated by reference in its entirety.

### TECHNICAL FIELD

This invention relates to pharmaceutical compositions.

### BACKGROUND

Active ingredients, such as drugs or pharmaceuticals, are delivered to patients in deliberate fashion. Delivery of drugs or pharmaceuticals using film transdermally or transmucosally can require that the drug or pharmaceutical permeate or otherwise cross a biological membrane in an effective and efficient manner.

### SUMMARY

In general, a method of administering a pharmaceutical composition, can include providing an oral film including: a polymeric matrix; a pharmaceutically active component including epinephrine or its prodrug in the polymeric matrix; and an adrenergic receptor interacter, positioning the film in an oral mucosa for a residence time and allowing the film to deliver the pharmaceutically active component.

In general, a method of administering a pharmaceutical composition, includes providing an oral film including: a polymeric matrix; a pharmaceutically active component including epinephrine or its prodrug in the polymeric matrix; and an adrenergic receptor interacter; positioning the film in an oral mucosa for a residence time; and allowing the film to deliver the pharmaceutically active component.

In certain embodiments, the residence time is no less than 4 minutes. In certain embodiments, the residence time is no less than 2 minutes. In certain embodiments, the residence time is no less than 0 minutes.

In certain embodiments, the residence time is no more than 2 minutes. In certain embodiments, the residence time is no more than 4 minutes. In certain embodiments, the residence time is no more than 6 minutes. In certain embodiments, the residence time is no more than 8 minutes.

In certain embodiments, the AUC for 0-10 minutes is equal to or greater than an intramuscular injection of a composition having a same pharmaceutically active component.

In certain embodiments, the AUC for 0-10 minutes is greater than 10 hr*pg/mL for a 4-minutes residence time. In certain embodiments, the AUC for 0-10 minutes is about 9 hr*pg/mL for a 2-minute residence time. In certain embodiments, the AUC for 0-10 minutes is about 9 hr*pg/mL for a 0-minute residence time.

In certain embodiments, the AUC for 0-20 minutes is equal to or greater than an intramuscular injection of a composition having a same pharmaceutically active component. In certain embodiments, the AUC for 0-20 minutes is about 50 hr*pg/mL for a 4-minute residence time.

In certain embodiments, the AUC for 0-20 minutes is about 45 hr*pg/mL for a 2-minute residence time. In certain embodiments, the AUC for 0-20 minutes is about 30 hr*pg/mL for a 0-minute residence time.

In certain embodiments, the AUC for 0-30 minutes is equal to or greater than an intramuscular injection of a composition having a same pharmaceutically active component.

In certain embodiments, the AUC for 0-30 minutes is about 80 hr*pg/mL for a 4-minute residence time. In certain embodiments, the AUC for 0-30 minutes is about 50 hr*pg/mL for a 2-minute residence time. In certain embodiments, the AUC for 0-30 minutes is about 50 hr*pg/mL for a 0-minute residence time.

In certain embodiments, therapeutic levels of active can be achieved in less than 15 minutes, less than 10 minutes or less than 5 minutes.

In certain embodiments, the composition is a film further comprising a polymeric matrix, the pharmaceutically active component being contained in the polymeric matrix.

In certain embodiments, the adrenergic receptor interacter includes a phenylpropanoid.

In certain embodiments, the adrenergic receptor interacter includes farnesol or Labrasol. In certain embodiments, the adrenergic receptor interacter includes linoleic acid. In certain embodiments, the adrenergic receptor interacter includes an aromatic compound, such as a substituted aromatic alcohol, for example, 8-hydroxy-p-cymene. In certain embodiments, the phenylpropanoid is eugenol or eugenol acetate. In certain embodiments, the phenylpropanoid is a cinnamic acid, cinnamic acid ester, cinnamic aldehyde or hydrocinnamic acid. In certain embodiments, the phenylpropanoid is chavicol. In certain embodiments, the phenylpropanoid is safrole. In certain embodiments, the adrenergic receptor interacter is a phytoextract. In certain embodiments, the phytoextract is synthetic or biosynthetic. In certain embodiments, the phytoextract further includes an essential oil extract of a clove plant.

In certain embodiments, the phytoextract further includes 40-95% eugenol. In certain embodiments, the adrenergic receptor interacter includes a terpenoid, terpene or a sesquiterpene.

In certain embodiments, the polymer matrix includes a polymer. In certain embodiments, the polymer is a water soluble polymer. In certain embodiments, the polymer includes a polyethylene oxide. In certain embodiments, the polymer includes a cellulosic polymer is selected from the group of: hydroxypropylmethyl cellulose, hydroxyethyl cellulose, hydroxyethylmethyl cellulose, hydroxypropyl cellulose, methylcellulose and carboxymethyl cellulose. In certain embodiments, the polymeric matrix comprises a cellulosic polymer, polyethylene oxide and polyvinyl pyrrolidone, polyethylene oxide and a polysaccharide, polyethylene oxide, hydroxypropyl methylcellulose and a polysaccharide, or polyethylene oxide, hydroxypropyl methylcellulose, polysaccharide and polyvinylpyrrolidone.

In certain embodiments, the polymeric matrix comprises at least one polymer selected from the group of: pullulan, polyvinyl pyrrolidone, polyvinyl alcohol, sodium alginate, polyethylene glycol, xanthan gum, tragancanth gum, guar gum, acacia gum, arabic gum, polyacrylic acid, methylmethacrylate copolymer, carboxyvinyl copolymers, starch, gelatin, ethylene oxide-propylene oxide co-polymers, collagen, albumin, poly-amino acids, polyphosphazenes, polysaccharides, chitin, chitosan, and derivatives thereof.

In certain embodiments, the method further comprises a stabilizer. In certain embodiments, the polymeric matrix comprises a dendritic polymer or a hyperbranched polymer.

In certain embodiments, the prodrug is dipifevrin.

In certain embodiments, the Tmax is 25 minutes or faster.

In certain embodiments, the Tmax is 20 minutes or faster.

In certain embodiments, the Tmax is 15 minutes or faster.

In certain embodiments, swallowing is permitted after the residence time. In certain embodiments, the pharmaceutically active component includes a prodrug of epinephrine.

In certain embodiments, the prodrug of epinephrine has a half-life of less than one minute.

In general, a method of administering epinephrine to a subject includes administering a composition including a prodrug of epinephrine to the oral cavity of the subject to deliver epinephrine through a digestive tract of the subject. In certain embodiments, the composition is an oral film. In certain embodiments, the composition further includes an adrenergic receptor interacter. In certain embodiments, the composition further includes a mixture of two adrenergic receptor interacters. In certain embodiments, the composition further includes a mixture of three adrenergic receptor interacters.

In certain embodiments, the oral film can include an absorbant component, an adsorbant component, or a desiccant. In certain embodiments, a suitable absorbant component, adsorbant component, or desiccant can include a silica, fumed silica or a mesoporous silica. In certain embodiments, the absorbant component, adsorbant component, or desiccant can be an amorphous silicon dioxide. In certain embodiments, the absorbant component, adsorbant component, or desiccant can include silicon dioxide variants such as a Cab-o-Sil or a Syloid product. In certain embodiments, the absorbant component, adsorbant component, or desiccant can be 1-15% by weight of the pharmaceutical composition. For example, the desiccant can be 2%, 3%, 4%, 5%, 6%, 7%, 8%, 9%, 10%, 11%, 12%, 13%, 14%, or 15% w/w of the pharmaceutical composition.

In certain embodiments, the silicon dioxide can have an average surface area of about 25-600 m²/g, for example 25 m²/g, 50 m²/g, 100 m²/g, 200 m²/g, 300 m²/g, 400 m²/g, 500 m²/g, or 600 m²/g. In certain embodiments, the silicon dioxide can have an average particle size of about 1-10 microns, for example 1 micron, 2 microns, 4 microns, 5 microns, 8 microns, about 10 microns or greater.

In certain embodiments, one or more oil components of the composition can be adsorbed on the desiccant. In certain embodiments, the absorbant component, adsorbant component, or desiccant can adsorb up to about 0.5 mL, 1.0 mL, 1.5 mL, or 2.0 mL of liquid per gram of desiccant.

In certain embodiments, the composition is formulated to be absorbed through the stomach, intestine or gastrointestinal tract. In certain embodiments, the composition is administered up to 2 minutes after consuming food. In certain embodiments, the composition is administered up to 4 minutes after consuming food. In certain embodiments, the composition is administered up to 6 minutes after consuming food. In certain embodiments, the composition is administered up to 8 minutes after consuming food. In certain embodiments, the composition is administered up to 10 minutes after consuming food. In certain embodiments, the composition is administered up to 15 minutes after consuming food. In certain embodiments, the composition is administered up to 20 minutes after consuming food. In certain embodiments, the composition is administered up to 30 minutes after consuming food. In certain embodiments, the composition is administered up to 60 minutes after consuming food. In certain embodiments, the composition is administered without food or drink. In certain embodiments, the composition is swallowed after an oral cavity residence or retention time. In certain embodiments, the composition is swallowed without a residence or retention time.

In certain embodiments, the residence or retention time is no less than 15 minutes. In certain embodiments, the residence or retention time is no less than 10 minutes. In certain embodiments, the residence or retention time is no less than 8 minutes. In certain embodiments, the residence or retention time is no less than 6 minutes. In certain embodiments, the residence or retention time is no less than 4 minutes. In certain embodiments, the residence or retention time is no less than 2 minutes. In certain embodiments, the residence or retention time is less than 2 minutes. In certain embodiments, the AUC for 0-10 minutes is greater than 10 hr*pg/mL for a 4-minutes residence time.

In certain embodiments, the AUC for 0-10 minutes is about 15 hr*pg/mL after food intake. In certain embodiments, the AUC for 0-10 minutes is about 2 hr*pg/mL with no residence time. In certain embodiments, the AUC for 0-20 minutes is about 50 hr*pg/mL for a 4-minute residence time. In certain embodiments, the AUC for 0-20 minutes is about 49 hr*pg/mL after food intake. In certain embodiments, the AUC for 0-20 minutes is about 28 hr*pg/mL with no residence time. In certain embodiments, the AUC for 0-30 minutes is about 79 hr*pg/mL for a 4-minute residence time. In certain embodiments, the AUC for 0-30 minutes is about 65 hr*pg/mL after food intake. In certain embodiments, the AUC for 0-30 minutes is about 73 hr*pg/mL with no residence time.

Other aspects, embodiments, and features will be apparent from the following description, the drawings, and the claims.

### BRIEF DESCRIPTION OF THE FIGURES

Referring to FIG. 1A, a Franz diffusion cell 100 includes a donor compound 101, a donor chamber 102, a membrane 103, sampling port 104, receptor chamber 105, stir bar 106, and a heater/circulator 107.

Referring to FIG. 1B, a pharmaceutical composition is a film 100 comprising a polymeric matrix 200, the pharmaceutically active component 300 being contained in the polymeric matrix.

The film can include a permeation enhancer 400.

Referring to FIGS. 2A and 2B, the graphs show the permeation of an active material from a composition.

Referring to FIG. 2A, this graph shows average amount of active material permeated vs. time, with 8.00 mg/mL epinephrine bitartrate and 4.4 mg/mL epinephrine base solubilized.

Referring to FIG. 2B, this graph shows average flux vs. time, with 8.00 mg/mL epinephrine bitartrate and 4.4 mg/mL epinephrine base solubilized.

Referring to FIG. 3, this graph shows ex-vivo permeation of epinephrine bitartrate as a function of concentration.

Referring to FIG. 4, this graph shows permeation of epinephrine bitartrate as a function of solution pH.

Referring to FIG. 5, this graph shows the influence of enhancers on permeation of epinephrine, indicated as amount permeated as a function of time.

Referring to FIGS. 6A and 6B, these graphs show the release of epinephrine on polymer platforms (6A) and the effect of enhancers on its release (6B), indicated as amount permeated (in pg) vs. time.

Referring to FIG. 7, this graph shows a pharmacokinetic model in the male Yucatan, miniature swine. The study compares a 0.3 mg Epipen, a 0.12 mg Epinephrine IV and a placebo film.

Referring to FIG. 8, this graph shows the impact of no enhancer on the concentration profiles of a 40 mg epinephrine film vs 0.3 mg Epipen.

Referring to FIG. 9, this graph shows the impact of Enhancer A (Labrasol) on the concentration profiles of a 40 mg epinephrine film vs 0.3 mg Epipen.

Referring to FIG. 10, this graph shows the impact of Enhancer L (clove oil) on the concentration profiles of two 40 mg Epinephrine films (10-1-1) and (11-1-1) vs. a 0.3 mg Epipen.

Referring to FIG. 11, this graph shows the impact of Enhancer L (clove oil) and film dimension (10-1-1 thinner, bigger film and 11-1-1 thicker, smaller film) on the concentration profiles of 40 mg Epinephrine films vs. a 0.3 mg Epipen.

Referring to FIG. 12, this graph shows the concentration profiles for varying doses of Epinephrine films in a constant matrix for Enhancer L (clove oil) vs. a 0.3 mg Epipen.

Referring to FIG. 13, this graph shows the concentration profiles for varying doses of epinephrine films in a constant matrix for Enhancer L (clove oil) vs. a 0.3 mg Epipen.

Referring to FIG. 14, this graph shows the concentration profiles for varying doses of epinephrine films in a constant matrix for Enhancer A (Labrasol) vs. a 0.3 mg Epipen.

Referring to FIG. 15, this graph shows the impact of Farnesol and Farnesol in combination with Linoleic Acid on plasma concentration profiles of 40 mg Epinephrine Films vs. a 0.3 mg Epipen.

Referring to FIG. 16, this graph shows the impact of Farnesol on plasma concentration profiles of 40 mg epinephrine films vs. a 0.3 mg Epipen.

Referring to FIG. 17, this graph shows the impact of Farnesol in combination with Linoleic Acid on plasma concentration profiles of 40 mg epinephrine films vs. a 0.3 mg Epipen.

Referring to FIG. 18, this graph shows the impact of Farnesol and Farnesol in combination with Linoleic Acid on plasma concentration profiles of 40 mg Epinephrine Films vs. a 0.3 mg Epipen.

Referring to FIG. 19, this graph shows the impact of Enhancer A (Labrasol) in combination with Enhancer L (clove oil) on the concentration profiles of a 40 mg Epinephrine film (also shown in FIG. 20), in logarithmic view.

Referring to FIG. 20, this graph shows the impact of Enhancer A (Labrasol) in combination with Enhancer L (clove oil) on the concentration profiles of a 40 mg Epinephrine film vs. the average data collected from 0.3 mg Epipens.

Referring to FIG. 21, this graph shows the impact of Enhancer A (Labrasol) in combination with Enhancer L (clove oil) on the concentration profiles of a 40 mg Epinephrine films, shown as separate animal subjects.

Referring to FIG. 22A and FIG. 22B, the data shows average epinephrine plasma concentration vs. time.

Referring to FIG. 23A and FIG. 23B, the data shows average dipivefrin plasma concentration vs. time.

Referring to FIG. 24, the data shows average plasma concentration of epinephrine administered as dipivefrin.

Referring to FIG. 25, the graph shows the conversion of dipivefrin to epinephrine.

Referring to FIG. 26, the data shows average epinephrine profiles across all matrix studies.

Referring to FIG. 27, the data shows additional data comparing plasma concentration of dipivefrin and epinephrine.

Referring to FIG. 28, this graph shows an epinephrine concentration vs. time profile of a film treated with phentolamine.

Referring to FIG. 29, this graph shows mean baseline adjusted epinephrine concentration over 0-2 hours with varying residence times.

Referring to FIG. 30, this graph shows mean baseline adjusted epinephrine concentration over 0-2 hours by treatment, comparing a 4 minute hold, direct swallowing (no residence time), and oral administration 2 minutes after food intake (peanut butter sandwich test).

Referring to FIG. 31A, this shows baseline adjusted epinephrine Cmax values for the 0.3 mg IM, prodrug and Epipen.

Referring to FIG. 31B, this shows the baseline adjusted epinephrine Cmax values within a timeframe critical to abate the cascade of anaphylaxis.

Referring to FIG. 31C, this shows the baseline adjusted epinephrine AUC over time.

Referring to FIG. 31D, this shows the baseline adjusted epinephrine Tmax over time.

Referring to FIG. 31E, this shows the fasted median Tmax compared to both 0.3 mg IM and the 0.3 mg EpiPen.

Referring to FIG. 32, shows a comparison of percentage of subjects to reach 100 μg/mL in 15 minutes.

Referring to FIG. 33A and FIG. 33B, these graphs represent mean baseline adjusted systolic blood pressure (SBP) over time.

Referring to FIG. 34, this shows the results from the repeat administration show all subjects receiving a second dose of AQST-109 received additional epinephrine.

Referring to FIG. 35, this shows the post hoc analysis of boxplots of baseline adjusted Cmax.

Referring to FIG. 36, this shows the impact of a manual residence study and improved pharmacokinetics.

Referring to FIG. 37A, this shows the study of a pharmaceutical film concentration over time in 240 minutes.

Referring to FIG. 37B, this shows this shows the study of a pharmaceutical film concentration over time in 15 minutes.

Referring to FIGS. 38A-38B, these show the Cmax of a pharmaceutical film compared with manual injection and available autoinjectors.

Referring to FIG. 38C, this shows the partial AUC's of a pharmaceutical film compared with manual injection and available autoinjectors.

Referring to FIG. 39, this shows the uncorrected epinephrine concentration (pg/mL) over time within 10 minutes.

Referring to FIG. 40A, this shows a study of mean adjusted changes in systolic blood pressure (SBP) after administering a pharmaceutical film compared with Epinephrine IM 0.3 mg (manual) and Epinephrine IM 0.3 mg (EpiPen),

Referring to FIG. 40B, this shows a study of mean adjusted changes in pulse (PR) after administering a pharmaceutical film compared with Epinephrine IM 0.3 mg (manual) and Epinephrine IM 0.3 mg (EpiPen)

Referring to FIG. 40C, this shows a repeat dose study comparing the concentration (pg/mL) over time of a pharmaceutical film and 0.3 mg IM Epinephrine.

Referring to FIG. 41A, this shows an exemplary process of making the pharmaceutical composition.

Referring to FIG. 41B depicts a comparison of individual adrenergic receptor interacters in terms of amount permeated as a function of time

Referring to FIG. 41C, this depicts ex vivo permeation as a function of time of different adrenergic receptor interacters.

Referring to FIG. 41D, this shows prodrug flux as a function of time different adrenergic receptor interacters.

Referring to FIG. 41E, this shows amount permeated as % of total load as function of time with different adrenergic receptor interacters.

Referring to FIG. 42A, this shows the impact of administration in concentration as a function of time (0-60 minutes).

Referring to FIG. 42B, this shows the impact of administration in concentration as a function of time (0-20 minutes).

Referring to FIG. 43, this shows pharmacokinetic parameters by treatment with the pivotal dosing instructions (pharmacokinetic parameters compared to a manual hold).

Referring to FIG. 44, this shows the administration effect on low responders, indicating an improved proportion of subjects reaching 100 μg/mL threshold.

Referring to FIG. 45, this shows the results of a bridge study with concordant outcomes using the same administration conditions of the pharmaceutical film.

Referring to FIG. 46, this shows a geometric mean plasma profile as a comparison of epinephrine plasma concentrations (in mg/mL) 10 minutes after dosing of the claimed pharmaceutical film and various approved auto-injectors at 0.3 mg.

Referring to FIG. 47, this shows a pAUC comparison of the claimed pharmaceutical film in 14 mg (top yellow line) and 12 mg (lower yellow line) compared to autoinjectors (shaded regions), showing that longer exposure data supports a dose selection of 12 mg based on upper bracket excursion with a 14 mg dose).

Referring to FIG. 48A, this shows Cmax (pg/mL) for the claimed pharmaceutical film bracketed by current FDA approved products (bars showing highest and lowest 75% of approved products).

Referring to FIG. 48B, this shows Tmax (minutes) for the claimed pharmaceutical film bracketed by current FDA approved products (bars showing highest and lowest 75% of approved products).

Referring to FIG. 49A, this shows the increase in systolic blood pressure within 2 minutes (following administration) between the claimed pharmaceutical film and autoinjectors.

Referring to FIG. 49B, this shows the increase in pulse within 2 minutes (following administration) between the claimed pharmaceutical film and autoinjectors.

Referring to FIG. 50, this shows the results from a study of the pharmacokinetic bracketing at a 12 mg dose between adrenaline 0.3 mg and Epipen 0.3 mg in healthy subjects.

FIG. 51 shows film delivery as a biocomparable to injector delivery.

FIG. 52 shows a graph of time to maximum concentration (Tmax) of film is significantly more consistent compared to autoinjectors.

FIG. 53 shows film exposure overtime (pAUC) is consistent and bracketed between injectors through 60 minutes.

FIG. 54 shows film reaches 100 μg/mL threshold as rapidly as EpiPen and faster than Adrenalin.

FIG. 55 shows film with comparators at later timepoints (PK Sustainability).

FIG. 56A-C shows graphs of blood pressure over time. FIG. 56A shows median changes in systolic blood pressure. FIG. 56B shows median change in diastolic blood pressure.

FIG. 56C shows median changes =in pulse.

FIG. 57 shows concentration with time with a repeat dose at 15 minutes.

FIG. 58 shows a graph of time post-dosing to maximum concentration of epinephrine.

FIG. 59 shows data for exposure over time.

FIG. 60 shows film subjects reach or exceed 100 μg/mL five minutes after second dosing.

FIG. 61A-C shows graphs of blood pressure over time. FIG. 61A shows median changes in systolic blood pressure. FIG. 61B shows median change in diastolic blood pressure.

FIG. 61C shows median changes in pulse.

FIG. 62 shows means baseline corrected epinephrine concentration over time by treatment with the film under different pH and temperatures conditions.

FIG. 63A shows the least squares geometric mean ratio and p-value for baseline corrected epinephrine primary PK parameters and secondary partial AUCs for the study shown in FIG. 62.

FIG. 63B shows the least squares geometric mean ratio and p-value for baseline corrected epinephrine primary PK parameters and secondary partial AUCs, for the study shown in FIG. 62.

### DETAILED DESCRIPTION

Mucosal surfaces, such as the oral mucosa, are a convenient route for delivering drugs to the body due to the fact that they are highly vascularized and permeable, providing increased bioavailability and rapid onset of action because it does not pass through the digestive system and thereby avoids first pass metabolism. In particular, the buccal and sublingual tissues offer advantageous sites for drug delivery because they are highly permeable regions of the oral mucosa, allowing drugs diffusing from the oral mucosa to have direct access to systemic circulation. This also offers increased convenience and therefore increased compliance in patients. For certain drugs, or pharmaceutically active components, a permeation enhancer can help to overcome the mucosal barrier and improve permeability. Permeation enhancers reversibly modulate the penetrability of the barrier layer in favor of drug absorption. Permeation enhancers facilitate transport of molecules through the epithelium. Absorption profiles and their rates can be controlled and modulated by a variety of parameters, such as but not limited to film size, drug loading, enhancer type/loading, polymer matrix release rate and mucosal residence time.

A pharmaceutical composition can be designed to deliver a pharmaceutically active component in a deliberate and tailored way. However, solubility and permeability of the pharmaceutically active component in vivo, in particular, in the mouth of a subject, can vary tremendously. A particular class of permeation enhancer can improve the uptake and bioavailability of the pharmaceutically active component in vivo. In particular, when delivered to the mouth via a film, the permeation enhancer can improve the permeability of the pharmaceutically active component through the mucosa and into the blood stream of the subject. The permeation enhancer can improve absorption rate and amount of the pharmaceutically active component by more than 5%, more than 10%, more than 20%, more than 30%, more than 40%, more than 50%, more than 60%, more than 70%, more than 80%, more than 90%, more than 100%, more than 150%, about 200% or more, or less than 200%, less than 150%, less than 100%, less than 90%, less than 80%, less than 70%, less than 60%, less than 50%, less than 40%, less than 30%, less than 20%, less than 10%, or less than 5%, or a combination of these ranges, depending on the other components in the composition.

In certain embodiments, a pharmaceutical composition has a suitable nontoxic, nonionic alkyl glycoside having a hydrophobic alkyl group joined by a linkage to a hydrophilic saccharide in combination with a mucosal delivery-enhancing agent selected from: (a) an aggregation inhibitory agent; (b) a charge-modifying agent; (c) a pH control agent; (d) a degradative enzyme inhibitory agent; (e) a mucolytic or mucus clearing agent; (f) a ciliostatic agent; (g) a membrane penetration-enhancing agent selected from: (i) a surfactant; (ii) a bile salt; (ii) a phospholipid additive, mixed micelle, liposome, or carrier; (iii) an alcohol; (iv) an enamine; (v) an NO donor compound; (vi) a long chain amphipathic molecule; (vii) a small hydrophobic penetration enhancer; (viii) sodium or a salicylic acid derivative; (ix) a glycerol ester of acetoacetic acid; (x) a cyclodextrin or beta-cyclodextrin derivative; (xi) a medium-chain fatty acid; (xii) a chelating agent; (xiii) an amino acid or salt thereof, (xiv) an N-acetylamino acid or salt thereof, (xv) an enzyme degradative to a selected membrane component; (ix) an inhibitor of fatty acid synthesis; (x) an inhibitor of cholesterol synthesis; and (xi) any combination of the membrane penetration enhancing agents recited in (i)-(x); (h) a modulatory agent of epithelial junction physiology; (i) a vasodilator agent; (j) a selective transport-enhancing agent; and (k) a stabilizing delivery vehicle, carrier, mucoadhesive, support or complex-forming species with which the compound is effectively combined, associated, contained, encapsulated or bound resulting in stabilization of the compound for enhanced transmucosal delivery, wherein the formulation of the compound with the transmucosal delivery-enhancing agents provides for increased bioavailability of the compound in blood plasma of a subject. Penetration enhancers have been described in J. Nicolazzo, et al., J. of Controlled Disease, 105 (2005) 1-15, which is incorporated by reference herein.

There are many reasons why the oral mucosa might be an attractive site for the delivery of therapeutic agents into the systemic circulation. Due to the direct drainage of blood from the buccal epithelium into the internal jugular vein first-pass metabolism in the liver and intestine may be avoided. First-pass effect can be a major reason for the poor bioavailability of some compounds when administered orally. Additionally, the mucosa lining the oral cavity is easily accessible, which ensures that a dosage form can be applied to the required site and can be removed easily in the case of an emergency. However, like the skin, the buccal mucosa acts as a barrier to the absorption of xenobiotics, which can hinder the permeation of compounds across this tissue. Consequently, the identification of safe and effective penetration enhancers has become a major goal in the quest to improve oral mucosal drug delivery.

A residence time refers to a time period in which a pharmaceutical composition, such as a pharmaceutical film, may be held in a given position on an oral mucosa before swallowing or without swallowing. The residence time can allow a fast dissolving dosage form to be a versatile platform for drug delivery, and can result in systemic and local action via oral, buccal, and/or sublingual, routes.

Chemical penetration enhancers are substances that control the permeation rate of a coadministered drug through a biological membrane. While extensive research has focused on obtaining an improved understanding of how penetration enhancers might alter intestinal and transdermal permeability, far less is known about the mechanisms involved in buccal and sublingual penetration enhancement.

The buccal mucosa delineates the inside lining of the cheek as well as the area between the gums and upper and lower lips and it has an average surface area of 100 cm². The surface of the buccal mucosa consists of a stratified squamous epithelium which is separated from the underlying connective tissue (lamina propria and submucosa) by an undulating basement membrane (a continuous layer of extracellular material approximately 1-2 μm in thickness). This stratified squamous epithelium consists of differentiating layers of cells which change in size, shape, and content as they travel from the basal region to the superficial region, where the cells are shed. There are approximately 40-50 cell layers, resulting in a buccal mucosa which is 500-600 μm thick.

Structurally the sublingual mucosa is comparable to the buccal mucosa but the thickness of this epithelium is 100-200 μm. This membrane is also non-keratinised and being relatively thinner has been demonstrated to be more permeable than buccal mucosa. Blood flow to the sublingual mucosal is slower compared with the buccal mucosa and is of the order of 1.0 ml/min-1/cm-2.

The permeability of the buccal mucosa is greater than that of the skin, but less than that of the intestine. The differences in permeability are the result of structural differences between each of the tissues. The absence of organized lipid lamellae in the intercellular spaces of the buccal mucosa results in greater permeability of exogenous compounds, compared to keratinized epithelia of the skin; while the increased thickness and lack of tight junctions results in the buccal mucosa being less permeable than intestinal tissue.

The primary barrier properties of the buccal mucosa have been attributed to the upper one-third to one-quarter of the buccal epithelium. Researchers have learned that beyond the surface epithelium, the permeability barrier of nonkeratinized oral mucosa could also be attributed to contents extruded from the membrane-coating granules into the epithelial intercellular spaces.

The intercellular lipids of the nonkeratinized regions of the oral cavity are of a more polar nature than the lipids of the epidermis, palate, and gingiva, and this difference in the chemical nature of the lipids may contribute to the differences in permeability observed between these tissues. Consequently, it appears that it is not only the greater degree of intercellular lipid packing in the stratum corneum of keratinized epithelia that creates a more effective barrier, but also the chemical nature of the lipids present within that barrier.

The existence of hydrophilic and lipophilic regions in the oral mucosa has led researchers to postulate the existence of two routes of drug transport through the buccal mucosa paracellular (between the cells) and transcellular (across the cells).

Since drug delivery through the buccal mucosa is limited by the barrier nature of the epithelium and the area available for absorption, various enhancement strategies are required in order to deliver therapeutically relevant amounts of drug to the systemic circulation. Various methods, including the use of chemical penetration enhancers, prodrugs, and physical methods may be employed to overcome the barrier properties of the buccal mucosa.

A chemical penetration enhancer, or absorption promoter, is a substance added to a pharmaceutical formulation in order to increase the membrane permeation or absorption rate of the coadministered drug, without damaging the membrane and/or causing toxicity. There have been many studies investigating the effect of chemical penetration enhancers on the delivery of compounds across the skin, nasal mucosa, and intestine. In recent years, more attention has been given to the effect of these agents on the permeability of the buccal mucosa. Since permeability across the buccal mucosa is considered to be a passive diffusion process the steady state flux (Jss) should increase with increasing donor chamber concentration (CD) according to Fick's first law of diffusion.

Surfactants and bile salts have been shown to enhance the permeability of various compounds across the buccal mucosa, both in vitro and in vivo. The data obtained from these studies strongly suggest that the enhancement in permeability is due to an effect of the surfactants on the mucosal intercellular lipids.

Fatty acids have been shown to enhance the permeation of a number of drugs through the skin, and this has been shown by differential scanning calorimetry and Fourier transform infrared spectroscopy to be related to an increase in the fluidity of intercellular lipids.

Additionally, pretreatment with ethanol has been shown to enhance the permeability of tritiated water and albumin across ventral tongue mucosa, and to enhance caffeine permeability across porcine buccal mucosa. There are also several reports of the enhancing effect of Azone® on the permeability of compounds through oral mucosa. Further, chitosan, a biocompatible and biodegradable polymer, has been shown to enhance drug delivery through various tissues, including the intestine and nasal mucosa.

Oral transmucosal drug delivery (OTDD) is the administration of pharmaceutically active agents through the oral mucosa to achieve systemic effects. Permeation pathways and predictive models for OTDD are described, e.g. in M. Sattar, Oral transmucosal drug delivery—Current status and future prospects, Int′l. Journal of Pharmaceutics, 47(2014) 498-506, which is incorporated by reference herein. OTDD continues to attract the attention of academic and industrial scientists. Despite limited characterization of the permeation pathways in the oral cavity compared with skin and nasal routes of delivery, recent advances in our understanding of the extent to which ionized molecules permeate the buccal epithelium, as well as the emergence of new analytical techniques to study the oral cavity, and the progressing development of in silico models predictive of buccal and sublingual permeation, prospects are encouraging.

In order to deliver broader classes of drugs across the buccal mucosa, reversible methods of reducing the barrier potential of this tissue should be employed. This requisite has fostered the study of penetration enhancers that will safely alter the permeability restrictions of the buccal mucosa. It has been shown that buccal penetration can be improved by using various classes of transmucosal and transdermal penetration enhancers such as bile salts, surfactants, fatty acids and their derivatives, chelators, cyclodextrins and chitosan. Among these chemicals used for the drug permeation enhancement, bile salts are the most common.

In vitro studies on enhancing effect of bile salts on the buccal permeation of compounds is discussed in Sevda Senel, Drug permeation enhancement via buccal route: possibilities and limitations, Journal of Controlled Release 72 (2001) 133-144, which is incorporated by reference herein. That article also discusses recent studies on the effects of buccal epithelial permeability of dihydroxy bile salts, sodium glycodeoxycholate (SGDC) and sodium taurodeoxycholate (TDC) and tri-hydroxy bile salts, sodium glycocholate (GC) and sodium taurocholate (TC) at 100 mM concentration including permeability changes correlated with the histological effects. Fluorescein isothiocyanate (FITC), morphine sulfate were each used as the model compound.

Chitosan has also been shown to promote absorption of small polar molecules and peptide/protein drugs through nasal mucosa in animal models and human volunteers. Other studies have shown an enhancing effect on penetration of compounds across the intestinal mucosa and cultured Caco-2 cells.

The permeation enhancer can be a phytoextract. A phytoextract can be an essential oil or composition including essential oils extracted by distillation of the plant material. In certain circumstances, the phytoextract can include synthetic analogues of the compounds extracted from the plant material (i.e., compounds made by organic synthesis). The phytoextract can include a phenylpropanoid, for example, phenyl alanine, eugenol, eugenol acetate, a cinnamic acid, a cinnamic acid ester, a cinnamic aldehyde, a hydrocinnamic acid, chavicol, 8-hydroxy-p-cymene, or safrole, or a combination thereof. The phytoextract can be an essential oil extract of a clove plant, for example, from the leaf, stem or flower bud of a clove plant. The clove plant can be Syzygium aromaticum. The phytoextract can include 20-95% eugenol, including 40-95% eugenol, including 60-95% eugenol, and for example, 80-95% eugenol. The extract can also include 5% to 15% eugenol acetate. The extract can also include caryophyllene. The extract can also include up to 2.1% α-humulen. Other volatile compounds included in lower concentrations in clove essential oil can be β-pinene, limonene, farnesol, benzaldehyde, 2-heptanone and ethyl hexanoate. Other permeation enhancers may be added to the composition to improve absorption of the drug. Suitable permeation enhancers include natural or synthetic bile salts such as sodium fusidate; glycocholate or deoxycholate and their salts; fatty acids and derivatives such as sodium laurate, oleic acid, oleyl alcohol, monoolein, and palmitoylcarnitine; chelators such as disodium EDTA, sodium citrate and sodium laurylsulfate, azone, sodium cholate, sodium 5-methoxysalicylate, sorbitan laurate, glyceryl monolaurate, octoxynonyl-9, laureth-9, polysorbates, sterols, or glycerides, such as caprylocaproyl polyoxylglycerides, e.g., Labrasol. The permeation enhancer can include phytoextract derivatives and/or monolignols. The permeation enhancer can also be a fungal extract.

Some natural products of plant origin have been known to have a vasodilatory effect. There are several mechanisms or modes by which plant-based products can evoke vasodilation. For review, see McNeill J. R. and Jurgens, T. M., Can. J. Physiol. Pharmacol. 84:803-821 (2006), which is incorporated by reference herein. Specifically, vasorelaxant effects of eugenol have been reported in a number of animal studies. See, e.g., Lahlou, S., et al., J. Cardiovasc. Pharmacol. 43:250-57 (2004), Damiani, C. E. N., et al., Vascular Pharmacol. 40:59-66 (2003), Nishijima, H., et al., Japanese J. Pharmacol. 79:327-334 (1998), and Hume W. R., J. Dent Res. 62(9):1013-15 (1983), each of which is incorporated by reference herein. Calcium channel blockade was suggested to be responsible for vascular relaxation induced by a plant essential oil, or its main constituent, eugenol. See, Interaminense L. R. L. et al., Fundamental & Clin. Pharmacol. 21: 497-506 (2007), which is incorporated by reference herein.

Fatty acids can be used as inactive ingredients in drug preparations or drug vehicles. Fatty acids can also be used as formulation ingredients due to their certain functional effects and their biocompatible nature. Fatty acid, both free and as part of complex lipids, are major metabolic fuel (storage and transport energy), essential components of all membranes and gene regulators. For review, see Rustan A. C. and Drevon, C. A., Fatty Acids: Structures and Properties, Encyclopedia of Life Sciences (2005), which is incorporated by reference herein. There are two families of essential fatty acids that are metabolized in the human body: ω-3 and ω-6 polyunsaturated fatty acids (PUFAs). If the first double bond is found between the third and the fourth carbon atom from the ω carbon, they are called ω-3 fatty acids. If the first double bond is between the sixth and seventh carbon atom, they are called ω-6 fatty acids. PUFAs are further metabolized in the body by the addition of carbon atoms and by desaturation (extraction of hydrogen). Linoleic acid, which is a ω-6 fatty acid, is metabolized to γ-linolenic acid, dihomo-γ-linolinic acid, arachidonic acid, adrenic acid, tetracosatetraenoic acid, tetracosapentaenoic acid and docosapentaenoic acid. α-linolenic acid, which is a ω-3 fatty acid is metabolized to octadecatetraenoic acid, eicosatetraenoic acid, eicosapentaenoic acid (EPA), docosapentaenoic acid, tetracosapentaenoic acid, tetracosahexaenoic acid and docosahexaenoic acid (DHA).

It has been reported that fatty acids, such as palmitic acid, oleic acid, linoleic acid and eicosapentaenoic acid, induced relaxation and hyperpolarization of porcine coronary artery smooth muscle cells via a mechanism involving activation of the Na⁺K⁺-APTase pump and the fatty acids with increasing degrees of cis-unsaturation had higher potencies. See, Pomposiello, S. I. et al., Hypertension 31:615-20 (1998), which is incorporated by reference herein. Interestingly, the pulmonary vascular response to arachidonic acid, a metabolite of linoleic acid, can be either vasoconstrictive or vasodilative, depending on the dose, animal species, the mode of arachidonic acid administration, and the tones of the pulmonary circulation. For example, arachidonic acid has been reported to cause cyclooxygenase-dependent and -independent pulmonary vasodilation. See, Feddersen, C. O. et al., J. Appl. Physiol. 68(5):1799-808 (1990); and see, Spannhake, E. W., et al., J. Appl. Physiol. 44:397-495 (1978) and Wicks, T. C. et al., Circ. Res. 38:167-71 (1976), each of which is incorporated by reference herein.

Many studies have reported effects of EPA and DHA on vascular reactivity after being administered as ingestible forms. Some studies found that EPA-DHA or EPA alone suppressed the vasoconstrictive effect of norepinephrine or increased vasodilatory responses to acetylcholine in the forearm microcirculation. See, Chin, J. P. F, et al., Hypertension 21:22-8 (1993), and Tagawa, H. et al., J Cardiovasc Pharmacol 33:633-40 (1999), each of which is incorporated by reference herein. Another study found that both EPA and DHA increased systemic arterial compliance and tended to reduce pulse pressure and total vascular resistance. See, Nestel, P. et al., Am J. Clin. Nutr. 76:326-30 (2002), which is incorporated by reference herein.

Meanwhile, a study found that DHA, but not EPA, enhanced vasodilator mechanisms and attenuates constrictor responses in forearm microcirculation in hyperlipidemic overweight men. See, Mori, T. A., et al., Circulation 102:1264-69 (2000), which is incorporated by reference herein. Another study found vasodilator effects of DHA on the rhythmic contractions of isolated human coronary arteries in vitro. See Wu, K.-T. et al., Chinese J. Physiol. 50(4):164-70 (2007), which is incorporated by reference herein.

The adrenergic receptors (or adrenoceptors) are a class of G protein-coupled receptors that are a target of catecholamines, especially norepinephrine (noradrenaline) and epinephrine (adrenaline). Epinephrine (adrenaline) interacts with both α- and β-adrenoceptors, causing vasoconstriction and vasodilation, respectively. Although a receptors are less sensitive to epinephrine, when activated, they override the vasodilation mediated by p-adrenoceptors because there are more peripheral al receptors than β-adrenoceptors. The result is that high levels of circulating epinephrine cause vasoconstriction. At lower levels of circulating epinephrine, β-adrenoceptor stimulation dominates, producing vasodilation followed by decrease of peripheral vascular resistance. The α1-adrenoreceptor is known for smooth muscle contraction, mydriasis, vasoconstriction in the skin, mucosa and abdominal vicera and sphincter contraction of the gastrointestinal (GI) tract and urinary bladder. The α1-adrenergic receptors are member of the Gq protein-coupled receptor superfamily. Upon activation, a heterotrimeric G protein, Gq, activates phospholipase C (PLC). The mechanism of action involves interaction with calcium channels and changing the calcium content in a cell. For review, see Smith R. S. et al., Journal of Neurophysiology 102(2): 1103-14 (2009), which is incorporated by reference herein. Many cells possess these receptors.

α1-adrenergic receptors can be a main receptor for fatty acids. For example, saw palmetto extract (SPE), widely used for the treatment of benign prostatic hyperplasia (BPH), has been reported to bind α1-adrenergic, muscarinic and 1,4-dihydropyridine (1,4-DHP) calcium channel antagonist receptors. See, Abe M., et al., Biol. Pharm. Bull. 32(4) 646-650 (2009), and Suzuki M. et al., Acta Pharmacologica Sinica 30:271-81 (2009), each of which is incorporated by reference herein. SPE includes a variety of fatty acids including lauric acid, oleic acid, myristic acid, palmitic acid and linoleic acid. Lauric acid and oleic acid can bind noncompetitively to α1-adrenergic, muscarinic and 1,4-DHP calcium channel antagonist receptors.

In certain embodiments, a permeation enhancer can be an adrenergic receptor interacter. An adrenergic receptor interacter refers to a compound or substance that modifies and/or otherwise alters the action of an adrenergic receptor. For example, an adrenergic receptor interacter can prevent stimulation of the receptor by increasing, or decreasing their ability to bind. Such interacters can be provided in either short-acting or long-acting forms. Certain short-acting interacters can work quickly, but their effects last only a few hours. Certain long-acting interacters can take longer to work, but their effects can last longer. The interacter can be selected and/or designed based on, e.g., on one or more of the desired delivery and dose, active pharmaceutical ingredient, permeation modifier, permeation enhancer, matrix, and the condition being treated. An adrenergic receptor interacter can be an adrenergic receptor blocker. The adrenergic receptor interacter can be a terpene (e.g. volatile unsaturated hydrocarbons found in the essential oils of plants, derived from units of isoprenes) or a C3-C22 alcohol or acid, preferably a C7-C18 alcohol or acid. In certain embodiments, the adrenergic receptor interacter can include an aromatic compound, for example, an aromatic alcohol. In certain embodiments, the aromatic alcohol can be a benzyl alcohol. In certain cirumstances, the benzyl alcohol can be a substituted benzyl alcohol. The substituted benzyl alcohol can be a C1-C4 substituted phenyl methanol, ethanol, or propanol. For example, the substituted benzyl alcohol can be 2-(4-methylphenyl)propan-2-ol, or cherry propanol. In certain embodiments, the adrenergic receptor interacter can include farnesol, linoleic acid, arachidonic acid, docosahexanoic acid, eicosapentanoic acid, and/or docosapentanoic acid. The acid can be a carboxylic acid, phosphoric acid, sulfuric acid, hydroxamic acid, or derivatives thereof. The derivative can be an ester or amide. For example, the adrenergic receptor interacter can be a fatty acid or fatty alcohol.

The C3-C22 alcohol or acid can be an alcohol or acid having a straight C3-C22 hydrocarbon chain, for example a C3-C22 hydrocarbon chain optionally containing at least one double bond, at least one triple bond, or at least one double bond and one triple bond; said hydrocarbon chain being optionally substituted with C1.4 alkyl, C₂₋₄ alkenyl, C₂₋₄ alkynyl, C₁₋₄ alkoxy, hydroxyl, halo, amino, nitro, cyano, C₃₋₅ cycloalkyl, 3-5 membered heterocycloalkyl, monocyclic aryl, 5-6 membered heteroaryl, C1.4 alkylcarbonyloxy, C₁₋₄ alkyloxycarbonyl, C₁₋₄ alkylcarbonyl, or formyl; and further being optionally interrupted by —O—, —N(R^{a})—, —N(R^{a})—C(O)—O—, —O—C(O)—N(R^{a})—, —N(R^{a})—C(O)—N(R^{b})—, or —O—C(O)—O—. Each of R^{a} and R^{b}, independently, is hydrogen, alkyl, alkenyl, alkynyl, alkoxy, hydroxylalkyl, hydroxyl, or haloalkyl. In certain embodiments, the alcohol or acid forming an ester can be a C3-C22 or C3-C16 group.

Fatty acids with a higher degree of unsaturation are effective candidates to enhance the permeation of drugs. Unsaturated fatty acids showed higher enhancement than saturated fatty acids, and the enhancement increased with the number of double bonds. See, A. Mittal, et al. Status of Fatty Acids as Skin Penetration Enhancers—A Review, Current Drug Delivery, 2009, 6, pp. 274-279, which is incorporated by reference herein. Position of double bond also affects the enhancing activity of fatty acids. Differences in the physicochemical properties of fatty acid which originate from differences in the double bond position most likely determine the efficacy of these compounds as skin penetration enhancers. Skin distribution increases as the position of the double bond is shifted towards the hydrophilic end. It has also been reported that fatty acid which has a double bond at an even number position more rapidly effects the perturbation of the structure of both the stratum corneum and the dermis than a fatty acid which has double bond at an odd number position. Cis-unsaturation in the chain can tend to increase activity.

An adrenergic receptor interacter can be a terpene. Hypotensive activity of terpenes in essential oils has been reported. See, Menezes I. A. et al., Z. Naturforsch. 65c:652-66 (2010), which is incorporated by reference herein. In certain embodiments, the permeation enhancer can be a sesquiterpene. Sesquiterpenes are a class of terpenes that consist of three isoprene units and have the empirical formula C₁₅H₂₄. Like monoterpenes, sesquiterpenes may be acyclic or contain rings, including many unique combinations. Biochemical modifications such as oxidation or rearrangement produce the related sesquiterpenoids. In certain embodiments, the adrenergic receptor interactor can be an active drug moiety, or a derivative or prodrug thereof, for example, an ester.

An adrenergic receptor interacter can be an unsaturated fatty acid such as linoleic acid. In certain embodiments, the permeation enhancer can be farnesol. Farnesol is a 15-carbon organic compound which is an acyclic sesquiterpene alcohol, which is a natural dephosphorylated form of farnesyl pyrophosphate. Under standard conditions, it is a colorless liquid. It is hydrophobic, and thus insoluble in water, but miscible with oils. Farnesol can be extracted from oils of plants such as citronella, neroli, cyclamen, and tuberose. It is an intermediate step in the biological synthesis of cholesterol from mevalonic acid in vertebrates. It has a delicate floral or weak citrus-lime odor and is used in perfumes and flavors. It has been reported that farnesol selectively kills acute myeloid leukemia blasts and leukemic cell lines in preference to primary hemopoietic cells. See, Rioja A. et al., FEBS Lett 467 (2-3): 291-5 (2000), which is incorporated by reference herein. Vasoactive properties of farnesyl analogues have been reported. See, Roullet, J.-B., et al., J. Clin. Invest., 1996, 97:2384-2390, which is incorporated by reference herein. Both Farnesol and N-acetyl-S-trans, trans-farnesyl-L-cysteine (AFC), a synthetic mimic of the carboxyl terminus of farnesylated proteins inhibited vasoconstriction in rat aortic rings.

In certain embodiments, an interacter can be an aporphine alkaloid. For example, an interacter can be a dicentrine.

In an embodiment, the pharmaceutical film can include at least one adrenergic receptor interacter. In certain embodiments, more than one adrenergic receptor interacters can be combined. In certain embodiments, two or three different adrenergic receptor interacters can be combined. A combination of adrenergic receptor interacters can modulate, alter or improve the pharmaceutical film's absorption profiles and organoleptic properties.

In general, an interacter can also be a vasodilator or a therapeutic vasodilator. Vasodilators are drugs that open or widen blood vessels. They are typically used to treat hypertension, heart failure and angina, but can be used to treat other conditions as well, including glaucoma for example. Some vasodilators that act primarily on resistance vessels (arterial dilators) are used for hypertension, and heart failure, and angina; however, reflex cardiac stimulation makes some arterial dilators unsuitable for angina. Venous dilators are very effective for angina, and sometimes used for heart failure, but are not used as primary therapy for hypertension. Vasodilator drugs can be mixed (or balanced) vasodilators in that they dilate both arteries and veins and therefore can have wide application in hypertension, heart failure and angina. Some vasodilators, because of their mechanism of action, also have other important actions that can in some cases enhance their therapeutic utility or provide some additional therapeutic benefit. For example, some calcium channel blockers not only dilate blood vessels, but also depress cardiac mechanical and electrical function, which can enhance their antihypertensive actions and confer additional therapeutic benefit such as blocking arrhythmias.

Vasodilator drugs can be classified based on their site of action (arterial versus venous) or by mechanism of action. Some drugs primarily dilate resistance vessels (arterial dilators; e.g., hydralazine), while others primarily affect venous capacitance vessels (venous dilators; e.g., nitroglycerine). Many vasodilator drugs have mixed arterial and venous dilator properties (mixed dilators; e.g., alpha-adrenoceptor antagonists, angiotensin converting enzyme inhibitors), such as phentolamine.

It is more common, however, to classify vasodilator drugs based on their primary mechanism of action. The figure to the right depicts important mechanistic classes of vasodilator drugs. These classes of drugs, as well as other classes that produce vasodilation, include: alpha-adrenoceptor antagonists (alpha-blockers); Angiotensin converting enzyme (ACE) inhibitors; Angiotensin receptor blockers (ARBs); beta₂-adrenoceptor agonists (O₂-agonists); calcium-channel blockers (CCBs); centrally acting sympatholytics; direct acting vasodilators; endothelin receptor antagonists; ganglionic blockers; nitrodilators; phosphodiesterase inhibitors; potassium-channel openers; renin inhibitors.

In general, the active or inactive components or ingredients can be substances or compounds that create an increased blood flow or flushing of the tissue to enable a modification or difference (increase or decrease) in transmucosal uptake of the API(s), and/or have a positive or negative heat of solution which are used as aids to modify (increase or decrease) transmucosal uptake.

### Sequence of Permeation Enhancer(s) and Active Pharmaceutical Ingredient(s)

The arrangement, order, or sequence of penetration enhancer(s) and active pharmaceutical ingredient(s)(API(s)) delivered to the desired mucosal surface can vary in order to deliver a desired pharmacokinetic profile. For example, one can apply the permeation enhancer(s) first by a film, by swab, spray, gel, rinse or by a first layer of a film then apply the API(s) by single film, by swab, or by a second layer of a film. The sequence can be reversed or modified, for example, by applying the API(s) first by film, by swab, or by a first layer of a film, and then applying the permeation enhancer(s) by a film, by swab, spray, gel, rinse or by a second layer of a film. In another embodiment, one may apply a permeation enhancer(s) by a film, and a drug by a different film. For example, the permeation enhancer(s) film positioned under a film containing the API(s), or the film containing the API(s) positioned under a film containing the permeation enhancer(s), depending on the desired pharmacokinetic profile.

For example, the penetration enhancer(s) can be used as a pretreatment alone or in combination with at least one API to precondition the mucosa for further absorption of the API(s). The treatment can be followed by another treatment with neat penetration enhancer(s) to follow the at least one API mucosal application. The pretreatment can be applied as a separate treatment (film, gel, solution, swab etc.) or as a layer within a multilayered film construction of one or more layers. Similarly, the pretreatment may be contained within a distinct domain of a single film, designed to dissolve and release to the mucosa prior to release of the secondary domains with or without penetration enhancer(s) or API(s). The active ingredient may then be delivered from a second treatment, alone or in combination with additional penetration enhancer(s). There may also be a tertiary treatment or domain that delivers additional penetration enhancer(s) and/or at least one API(s) or prodrug(s), either at a different ratio relative to each other or relative to the overall loading of the other treatments. This allows a custom pharmacokinetic profile to be obtained. In this way, the product may have single or multiple domains, with penetration enhancer(s) and API(s) that can vary in mucosal application order, composition, concentration, or overall loading that leads to the desired absorption amounts and/or rates that achieve the intended pharmacokinetic profile and/or pharmacodynamic effect.

The film format can be oriented such that no distinct sides, or such that the film has at least one side of a multiple layer film where the edges are co-terminus (having or meeting at a shared border or limit).

The pharmaceutical composition can be a chewable or gelatin based dosage form, spray, gum, gel, cream, tablet, liquid or film. The composition can include textures, for example, at the surface, such as microneedles or micro-protrusions. Recently, the use of micron-scale needles in increasing skin permeability has been shown to significantly increase transdermal delivery, including and especially for macromolecules. Most drug delivery studies have emphasized solid microneedles, which have been shown to increase skin permeability to a broad range of molecules and nanoparticles in vitro. In vivo studies have demonstrated delivery of oligonucleotides, reduction of blood glucose level by insulin, and induction of immune responses from protein and DNA vaccines. For such studies, needle arrays have been used to pierce holes into skin to increase transport by diffusion or iontophoresis or as drug carriers that release drug into the skin from a microneedle surface coating. Hollow microneedles have also been developed and shown to microinject insulin to diabetic rats. To address practical applications of microneedles, the ratio of microneedle fracture force to skin insertion force (i.e. margin of safety) was found to be optimal for needles with small tip radius and large wall thickness. Microneedles inserted into the skin of human subjects were reported as painless. Together, these results suggest that microneedles represent a promising technology to deliver therapeutic compounds into the skin for a range of possible applications. Using the tools of the microelectronics industry, microneedles have been fabricated with a range of sizes, shapes and materials. Microneedles can be, for example, polymeric, microscopic needles that deliver encapsulated drugs in a minimally invasive manner, but other suitable materials can be used.

Applicants have found that microneedles could be used to enhance the delivery of drugs through the oral mucosa, particularly with the claimed compositions. The microneedles create micron sized pores in the oral mucosa which can enhance the delivery of drugs across the mucosa. Solid, hollow or dissolving microneedles can be fabricated out of suitable materials including, but not limited to, metal, polymer, glass and ceramics. The microfabrication process can include photolithography, silicon etching, laser cutting, metal electroplating, metal electro polishing and molding. Microneedles could be solid which is used to pretreat the tissue and are removed before applying the film. The drug loaded polymer film described in this application can be used as the matrix material of the microneedles itself. These films can have microneedles or micro protrusions fabricated on their surface which will dissolve after forming microchannels in the mucosa through which drugs can permeate.

The term “film” can include films and sheets, in any shape, including rectangular, square, or other desired shape. A film can be any desired thickness and size. In preferred embodiments, a film can have a thickness and size such that it can be administered to a user, for example, placed into the oral cavity of the user. A film can have a relatively thin thickness of from about 0.0025 mm to about 0.250 mm, or a film can have a somewhat thicker thickness of from about 0.250 mm to about 1.0 mm. For some films, the thickness may be even larger, i.e., greater than about 1.0 mm or thinner, i.e., less than about 0.0025 mm. A film can be a single layer or a film can be multi-layered, including laminated or multiple cast films. A permeation enhancer and pharmaceutically active component can be combined in a single layer, each contained in separate layers, or can each be otherwise contained in discrete regions of the same dosage form. In certain embodiments, the pharmaceutically active component contained in the polymeric matrix can be dispersed in the matrix. In certain embodiments, the permeation enhancer being contained in the polymeric matrix can be dispersed in the matrix.

Oral dissolving films can fall into three main classes: fast dissolving, moderate dissolving and slow dissolving. Oral dissolving films can also include a combination of any of the above categories. Fast dissolving films can dissolve in about 1 second to about 30 seconds in the mouth, including more than 1 second, more than 5 seconds, more than 10 seconds, more than 20 seconds, and less than 30 seconds. Moderate dissolving films can dissolve in about 1 to about 30 minutes in the mouth including more than 1 minute, more than 5 minutes, more than 10 minutes, more than 20 minutes or less than 30 minutes, and slow dissolving films can dissolve in more than 30 minutes in the mouth. As a general trend, fast dissolving films can include (or consist of) low molecular weight hydrophilic polymers (e.g., polymers having a molecular weight between about 1,000 to 9,000 daltons, or polymers having a molecular weight up to 200,000 daltons). In contrast, slow dissolving films generally include high molecular weight polymers (e.g., having a molecular weight in millions). Moderate dissolving films can tend to fall in between the fast and slow dissolving films.

It can be preferable to use films that are moderate dissolving films. Moderate dissolving films can dissolve rather quickly, but also have a good level of mucoadhesion. Moderate dissolving films can also be flexible, quickly wettable, and are typically non-irritating to the user. Such moderate dissolving films can provide a quick enough dissolution rate, most desirably between about 1 minute and about 20 minutes, while providing an acceptable mucoadhesion level such that the film is not easily removable once it is placed in the oral cavity of the user. This can ensure delivery of a pharmaceutically active component to a user.

The absorption of the active pharmaceutical ingredient through the mucosa can be governed by several factors including the ability of the molecule to permeate and traverse the oral mucosa to reach the systemic or vascular system. Despite significant improvements to the permeation rate, through the incorporation of a penetration enhancer into the formulation, only a portion of the active pharmaceutical ingredient is delivered transmucosally. The remainder of the the active pharmaceutical ingredient is washed away through salivary flow or swallowed and drains into the other mucosal areas such as the pharyngeal, esophogeal and gastrointestinal tract where it is absorbed into the body, for example, by a transpharyngeal, transesophogeal or transgastrointestinal mechanism. The combination of one or more of these absorption routes allows a composition such as a spray to deliver faster the active pharmaceutical ingredient (e.g. epinephrine) blood levels than can be achieved orally alone but due to the high oral bioavailability, also ensures a complete dose is delivered upon every application. The swallowed portion of the the active pharmaceutical ingredient however is also exposed to variations in the absorption rate due to, e.g., a food effect during oral administration.

The pharmaceutical composition can be formulated to deliver the active pharmaceutical ingredient via at least a gastrointestinal route. The active pharmaceutical ingredient can also be delivered via at least a pharyngeal route. Such delivery includes the part of the throat behind the mouth and nasal cavity, and above the esophagus and trachea (the tubes going down to the stomach and the lungs). This mode of delivery can result in rapid drug uptake. In certain embodiments, the active pharmaceutical ingredient is delivered via at least a esophageal route. This delivery includes the esophagus, a muscular tube connecting the throat (pharynx) with the stomach. Dosage forms that adhere to the esophageal mucosa and prolong contact have been investigated to improve the efficacy of locally acting agents. These modes of delivery can also result in rapid drug uptake, a large surface area for solute transport, improved drug bioavailability, and noninvasive manner of administering the drug.

A pharmaceutical composition can include one or more pharmaceutically active components. The pharmaceutically active component can be a single pharmaceutical component or a combination of pharmaceutical components. The pharmaceutically active component can be an anti-inflammatory analgesic agent, a steroidal anti-inflammatory agent, an antihistamine, a local anesthetic, a bactericide, a disinfectant, a vasoconstrictor, a hemostatic, a chemotherapeutic drug, an antibiotic, a keratolytic, a cauterizing agent, an antiviral drug, an antirheumatic, an antihypertensive, a bronchodilator, an anticholinergic, an anti-anxiety drug, an antiemetic compound, a hormone, a peptide, a protein or a vaccine. The pharmaceutically active component can be the compound, pharmaceutically acceptable salt of a drug, a prodrug, a derivative, a drug complex or analog of a drug. The term “prodrug” refers to a biologically inactive compound that can be metabolized in the body to produce a biologically active drug. For example, the pharmaceutically active component can be an ester of epinephirine, for example, dipivefrin. See, e.g., J. Anderson, et al., Site of ocular hydrolysis of a prodrug, dipivefrin, and a comparison of its ocular metabolism with that of the parent compounds, epinephrine, Invest., Ophthalmol. Vis. Sci. July 1980. The time it takes to convert 50% of the prodrug by an enzyme or multiple enzymes to yield neat epinephrine systemically in humans is referred to as the half-life. In certain embodiments, the half-life is less than 1 minute.

In certain embodiments, the composition including a prodrug includes more than one prodrug with each prodrug being a derivative of a pharmaceutically active ingredient. In some of these embodiments, one of the prodrugs is dipivefrin.

In certain embodiments, the first prodrug is a first ester of epinephrine and the second prodrug is a second ester of epinephrine, the first ester of epinephrine and the second ester of epinephrine being different.

In certain embodiments, the prodrug is a compound of formula (I), wherein

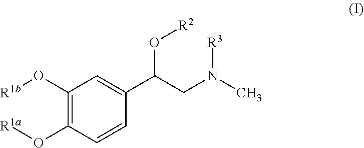

each of R^{1a}, R^{1b}, R² and R³, independently, can be H, C1-C16 acyl, alkyl aminocarbonyl, alkyloxycarbonyl, phenacyl, sulfate or phosphate, or R^{1a} and R^{1b} together, R^{1a} and R² together, R^{1a} and R³ together, R^{1b} and R² together, R^{1b} and R³ together, or R² and R³ together form a cyclic structure including a dicarbonyl, disulfate or diphosphate moiety, provided that one of R^{1a}, R^{1b}, R² and R³ is not H, or a pharmaceutically acceptable salt thereof.

In certain embodiments, R² and R³ are H and each R^{1a} and R^{1b}, independently, can be ethanoyl, n-propanoyl, isopropanoyl, n-butanoyl, isobutanoyl, sec-butanoyl, tert-butanoyl, n-pentanoyl, isopentanoyl, sec-pentanoyl, tert-pentanoyl, or neopentanoyl. In some embodiments, both of R^{1a} and R^{1b} can be ethanoyl, n-propanoyl, isopropanoyl, n-butanoyl, isobutanoyl, sec-butanoyl, tert-butanoyl, n-pentanoyl, isopentanoyl, sec-pentanoyl, tert-pentanoyl, or neopentanoyl. In some embodiments, one of R^{1a} and R^{1b} can be ethanoyl, n-propanoyl, isopropanoyl, n-butanoyl, isobutanoyl, sec-butanoyl, tert-butanoyl, n-pentanoyl, isopentanoyl, sec-pentanoyl, tert-pentanoyl, or neopentanoyl.

Exemplary prodrugs are provided in the table below.

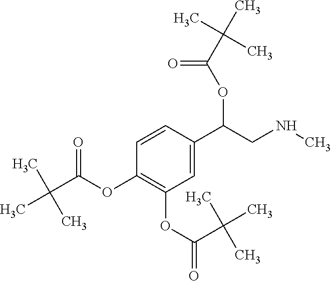 [SMILES: CNCC(OC(=O)C(C)(C)C)c1ccc(OC(=O)C(C)(C)C)c(OC(=O)C(C)(C)C)c1]

AQEP-01

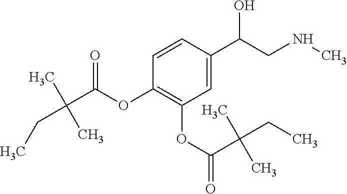 [SMILES: CCC(C)(C)C(=O)Oc1ccc(C(O)CNC)cc1OC(=O)C(C)(C)CC]

AQEP-02

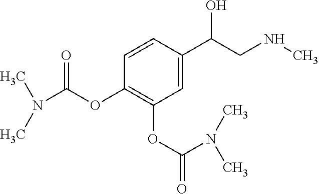

AQEP-03

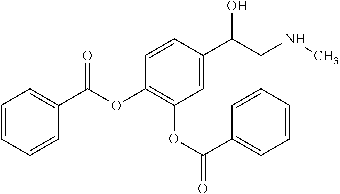 [SMILES: CNCC(O)c1ccc(OC(=O)c2ccccc2)c(OC(=O)c2ccccc2)c1]

AQEP-04

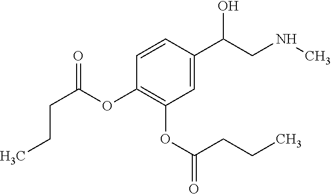 [SMILES: CCCC(=O)Oc1ccc(C(O)CNC)cc1OC(=O)CCC]

AQEP-05

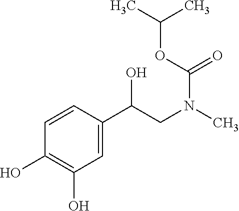 [SMILES: CC(C)OC(=O)N(C)CC(O)c1ccc(O)c(O)c1]

AQEP-06

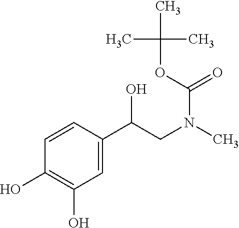 [SMILES: CN(CC(O)c1ccc(O)c(O)c1)C(=O)OC(C)(C)C]

AQEP-07

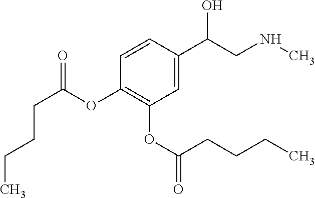 [SMILES: CCCCC(=O)Oc1ccc(C(O)CNC)cc1OC(=O)CCCC]

AQEP-08

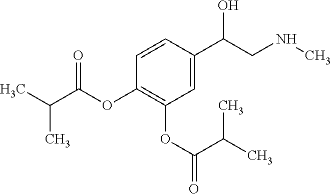 [SMILES: CNCC(O)c1ccc(OC(=O)C(C)C)c(OC(=O)C(C)C)c1]

AQEP-09

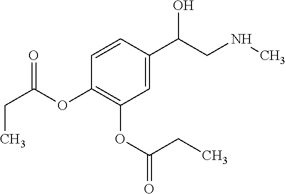 [SMILES: CCC(=O)Oc1ccc(C(O)CNC)cc1OC(=O)CC]

AQEP-10

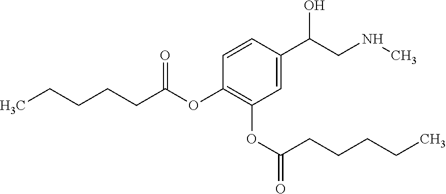 [SMILES: CCCCCC(=O)Oc1ccc(C(O)CNC)cc1OC(=O)CCCCC]

AQEP-11

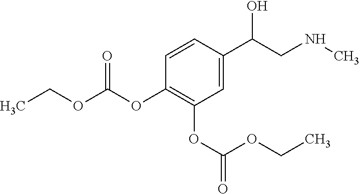 [SMILES: CCOC(=O)Oc1ccc(C(O)CNC)cc1OC(=O)OCC]

AQEP-12

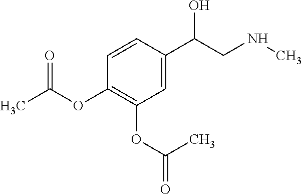 [SMILES: CNCC(O)c1ccc(OC(C)=O)c(OC(C)=O)c1]

AQEP-13

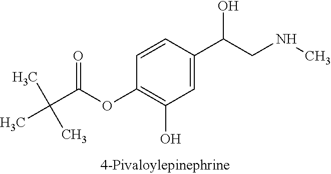 [SMILES: CNCC(O)c1ccc(OC(=O)C(C)(C)C)c(O)c1]

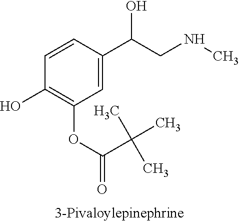 [SMILES: CNCC(O)c1ccc(O)c(OC(=O)C(C)(C)C)c1]

AQEP-14

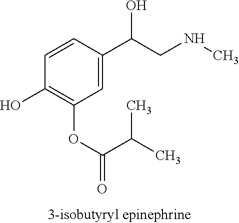 [SMILES: CNCC(O)c1ccc(O)c(OC(=O)C(C)C)c1]

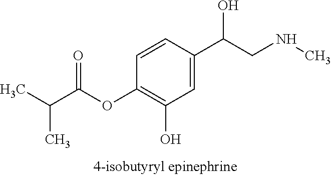 [SMILES: CNCC(O)c1ccc(OC(=O)C(C)C)c(O)c1]

AQEP-15

Administering epinephrine as a prodrug such as dipivefrin, or prodrugs AQEP-03, AQEP-04, AQEP-05, AQEP-06, AQEP-07, AQEP-08, AQEP-09, AQEP-10, AQEP-11, AQEP-12, AQEP-13, AQEP-14 or AQEP-15 confer certain advantages. For one, dipivefrin and prodrugs AQEP-03, AQEP-04, AQEP-05, AQEP-06, AQEP-07, AQEP-08, AQEP-09, AQEP-10, AQEP-11, AQEP-12, AQEP-13, AQEP-14 and AQEP-15 are lipophilic and therefore has a higher permeation through a mucosa. Dipivefrin and prodrugs AQEP-03, AQEP-04, AQEP-05, AQEP-06, AQEP-07, AQEP-08, AQEP-09, AQEP-10, AQEP-11, AQEP-12, AQEP-13, AQEP-14 and AQEP-15 each have a longer plasma half-life due to higher protein binding. Dipivefrin is capable of sustained blood levels, and has a reduced interaction with α-receptors, therefore minimizing or eliminating unwanted or harmful vasoconstriction. Prodrugs, for example, AQEP-09, can exhibit higher binding affinity for α- and β-receptors, with binding and activation profiles that are more similar to epinephrine than dipivefrin. Other prodrugs, and combinations of prodrugs, can exhibit binding affinities for α- and β-receptors that favor one or more receptor, similar to or different from epinephrine.

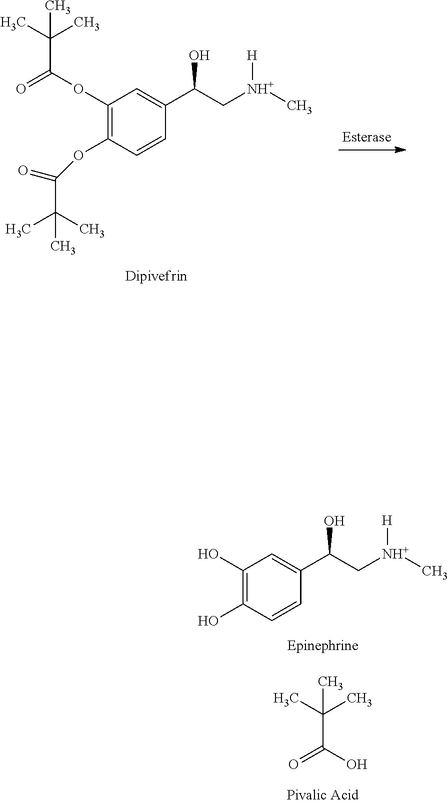

Dipivefrin or prodrugs AQEP-03, AQEP-04, AQEP-05, AQEP-06, AQEP-07, AQEP-08, AQEP-09, AQEP-10, AQEP-11, AQEP-12, AQEP-13, AQEP-14 or AQEP-15, alone or in combination, can be delivered in sublingual film in a similar manner as with epinephrine delivered by other methods, including injection.

The compound of formula I can be a pharmaceutically acceptable salt. The pharmaceutically acceptable salt can be an acid addition salt or a base addition salt. Acid addition salts can be prepared by reacting the purified compound in its free-based form with a suitable organic or inorganic acid and isolating the salt thus formed. Examples of pharmaceutically acceptable acid addition salts include, without limitations, salts of an amino group formed with inorganic acids such as hydrochloric acid, hydrobromic acid, phosphoric ac-id, sulfuric acid and perchloric acid, or with organic acids such as acetic acid, oxalic acid, maleic acid, tartaric acid, citric acid, succinic acid or malonic acid. Base addition salts can be prepared by reacting the purified compound in its acid form with a suitable organic or inorganic base and isolating the salt thus formed. Such salts include, without limitations, alkali metal (e.g., sodium, lithium, and potassium), alkaline earth metal (e.g., magnesium and calcium), ammonium, alkylammonium, substituted alkylammonium and N*(C₁₋₄alkyl)₄ salts. The alkyl can be a hydroxyalkyl. Other pharmaceutically acceptable salts of the compound can include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, and valerate salts.

To deliver epinephrine, a class of prodrug compounds was tested having modifications made to the R^{1a}, R^{1b}, R² and R³ groups of epinephrine as shown below. The R^{1a} and R^{1b} groups can include esters, amides, carbonates and carbamates, orthoesters or acetals. The groups can include for example, alkyl esters, chloroalkyl esters, amides, alkyl amides, chloroalkyl amides. The R² groups can include benzylic alcohol modification. The R³ group can include amine modification or oxazolidines. An ideal prodrug would have one or more of the following attributes, is biologically acceptable, penetrates a tissue, is stable and converts in the body, tissue or blood. In some cases, the prodrug may not need any permeation enhancers at all but rather permeate sufficiently by itself. The conversion of the prodrug to active is not predictable based on chain length of the R^{1a}, R^{1b}, R² and R³ groups. In particular, a tertiary group at the second atom of the R^{1a}, R^{1b}, R² or R³ group. The permeation of the prodrug is also unpredictable based on the R^{1a}, R^{1b}, R² and R³ groups.

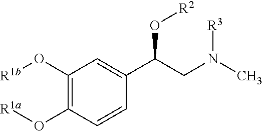

In general, a method of treating a medical condition can include administering a prodrug from a matrix, the prodrug being converted at controlled rate, for example, at a rate of 20 pg/ml to about 40 ng/ml of active compound. The prodrug can be converted at a rate where the active compound cannot be detected in plasma. For example, the prodrug conversion to active compound can be slow and sustained. In certain circumstances, the prodrug conversion to active can be less than 240 minutes, less than 180 minutes, less than 120 minutes, less than 60 minutes, or less than 30 minutes. In other circumstances, the prodrug conversion can be a slow conversion, for example, providing active compound exposure for 4 hours, 6 hours, 8 hours, 12 hours, 18 hours, 24 hours, or longer. For example, the composition can supply active compound for daily, 24 hour dosing.

In certain circumstances, the composition can be administered using microneedles or nanofibers. This type of administration can regulate delivery rates of the prodrug. In other circumstances, this type of administration can be used to direct the prodrug to specific structures in the skin tissue or other tissues.

The prodrug can be converted to 200 pg/ml to about 1200 pg/ml of active compound in less than 120 minutes. In certain embodiments, prodrug is converted to 200 pg/ml to about 1200 pg/ml of active compound in less than 100 minutes. The prodrug can also be converted to 200 pg/ml to about 600 pg/ml of active compound in less than 60 minutes. In certain embodiments, the prodrug is converted to 200 pg/ml to about 600 pg/ml of active compound in less than 45 minutes. In certain embodiments, the prodrug is converted to 200 pg/ml to about 600 pg/ml of active compound in less than 30 minutes.

In certain embodiments, the prodrug converts to create a sustained concentration of 200 pg/ml to about 600 pg/ml of active compound.

In certain embodiments, less than 100% of the prodrug is converted. In other embodiments, 100% of the prodrug is converted.

In general, a method of treating a medical condition comprising administering a prodrug, the prodrug being converted to produce a concentration of active from 20 pg/ml to about 40 ng/ml of active compound in less than 240 minutes and in which 100% of prodrug is converted. In certain circumstances, a method of treating a medical condition comprising administering a prodrug from a matrix, the prodrug being converted to produce a concentration of active from 20 pg/ml to about 40 ng/ml of active compound in less than 240 minutes and in which less than 100% of prodrug is converted. The prodrug can be administered from a matrix.

In certain embodiments, the prodrug can produce therapeutic levels over 100 pg/ml of epinephrine for a duration of at least 1 hour. In certain embodiments, the prodrug can produce therapeutic levels over 100 pg/ml of epinephrine for a duration of at least 2 hours. In certain embodiments, the prodrug produces therapeutic levels over 100 pg/ml of epinephrine for a duration of at least 3 hours. In certain embodiments, the prodrug produces therapeutic levels over 100/ml pg of epinephrine for a duration of at least 4 hours.

A prodrug can be metabolized, for example by hydrolysis. Metabolism can occur through enzymatic conversion, for example through hydrolytic enzymes, which convert a prodrug into an active compound. A prodrug can be converted at various times and in various ways in the body. A prodrug can be designed based on a targeted approach for in any suitable manner based on where and when conversion is desired. In some instances, prodrug conversion can occur systemically (e.g. in circulation). In some situations, prodrug conversion occurs intracellularly (e.g., antiviral nucleoside analogs, lipid-lowering statins). In some situations, prodrug conversion can occur extracellularly, for examples in digestive fluids or other extracellular body fluids).

In certain embodiments, at least half of the administered prodrug is converted in less than 240 minutes. In certain embodiments, at least half of the administered prodrug is converted in less than 120 minutes. In other embodiments, at least half of the administered prodrug is converted in less than 60 minutes. In other embodiments, at least half of the administered prodrug is converted in less than 30 minutes. In other embodiments, at least half of the administered prodrug is converted in less than 15 minutes. In other embodiments, at least half of the administered prodrug is converted in less than 10 minutes. In other embodiments, at least half of the administered prodrug is converted in less than 5 minutes. In other embodiments, at least half of the administered prodrug is converted in less than 1 minute.

For example, when the prodrug is an ester of epinephrine, the half-life of hydrolysis of the ester to form epinephrine in-vivo in a human subject can be less than one minute. For example, a review of pharmacokinetic data from a study in which 12 mg of an ester of epinephrine is delivered by a sublingual film as described herein, epinephrine plasma levels clearly indicate significant systemic absorption of epinephrine, however, the requisite parent prodrug concentration was undetectable. The absence of detectable prodrug in the plasma indicates a conversion half life of the prodrug that is not able to be calculated by traditional pharmacokinetic techniques. The detection limit of the prodrug was 2 pg/ml. By inferring the time between blood sampling timepoints (5 minutes) around the observed Cmax, it can be reasonably concluded that the half life must be less than 1 minute and is very likely to be less than 30 seconds based on the data, in order to adequately clear the requisite prodrug level before the next timepoint.

The half-life of hydrolysis of a prodrug or an ester of epinephrine in-vivo in a human subject should not be confused with the release or dissolution of epinephrine hydrochloride from a film into 500 mL of simulated saliva as described in Alayoubi et al. Pharm. Dev. Technol. 2017, 22(8), 1012-1016, which is incorporated by reference herein. Note also that the Alayoubi report did not investigate the ability of a film to deliver epinephrine in vivo and did not involve a prodrug or the hydrolysis of any prodrug systemically.

In some embodiments, more than one pharmaceutically active component may be included in the film. The pharmaceutically active components can be ace-inhibitors, anti-anginal drugs, anti-arrhythmias, anti-asthmatics, anti-cholesterolemics, analgesics, anesthetics, anti-convulsants, anti-depressants, anti-diabetic agents, anti-diarrhea preparations, antidotes, anti-histamines, anti-hypertensive drugs, anti-inflammatory agents, anti-lipid agents, anti-manics, anti-nauseants, anti-stroke agents, anti-thyroid preparations, amphetamines, anti-tumor drugs, anti-viral agents, acne drugs, alkaloids, amino acid preparations, anti-tussives, anti-uricemic drugs, anti-viral drugs, anabolic preparations, systemic and non-systemic anti-infective agents, anti-neoplastics, anti-parkinsonian agents, anti-rheumatic agents, appetite stimulants, blood modifiers, bone metabolism regulators, cardiovascular agents, central nervous system stimulates, cholinesterase inhibitors, contraceptives, decongestants, dietary supplements, dopamine receptor agonists, endometriosis management agents, enzymes, erectile dysfunction therapies, fertility agents, gastrointestinal agents, homeopathic remedies, hormones, hypercalcemia and hypocalcemia management agents, immunomodulators, immunosuppressives, migraine preparations, motion sickness treatments, muscle relaxants, obesity management agents, osteoporosis preparations, oxytocics, parasympatholytics, parasympathomimetics, prostaglandins, psychotherapeutic agents, respiratory agents, sedatives, smoking cessation aids, sympatholytics, tremor preparations, urinary tract agents, vasodilators, laxatives, antacids, ion exchange resins, anti-pyretics, appetite suppressants, expectorants, anti-anxiety agents, anti-ulcer agents, anti-inflammatory substances, coronary dilators, cerebral dilators, peripheral vasodilators, psycho-tropics, stimulants, anti-hypertensive drugs, vasoconstrictors, migraine treatments, antibiotics, tranquilizers, anti-psychotics, anti-tumor drugs, anti-coagulants, anti-thrombotic drugs, hypnotics, anti-emetics, anti-nauseants, anti-convulsants, neuromuscular drugs, hyper- and hypo-glycemic agents, thyroid and anti-thyroid preparations, diuretics, anti-spasmodics, uterine relaxants, anti-obesity drugs, erythropoietic drugs, anti-asthmatics, cough suppressants, mucolytics, DNA and genetic modifying drugs, diagnostic agents, imaging agents, dyes, or tracers, and combinations thereof.

For example, the pharmaceutically active component can be buprenorphine, naloxone, acetaminophen, riluzole, clobazam, Rizatriptan, propofol, methyl salicylate, monoglycol salicylate, aspirin, mefenamic acid, flufenamic acid, indomethacin, diclofenac, alclofenac, diclofenac sodium, ibuprofen, ketoprofen, naproxen, pranoprofen, fenoprofen, sulindac, fenclofenac, clidanac, flurbiprofen, fentiazac, bufexamac, piroxicam, phenylbutazone, oxyphenbutazone, clofezone, pentazocine, mepirizole, tiaramide hydrochloride, hydrocortisone, predonisolone, dexarnethasone, triamcinolone acetonide, fluocinolone acetonide, hydrocortisone acetate, predonisolone acetate, methylpredonisolone, dexamethasone acetate, betamethasone, betamethasone valerate, flumetasone, fluorometholone, beclomethasone diproprionate, fluocinonide, edaravone, lurasidone, esomeprazole, lumateperone, naldmedine, doxylamine, pyridoxine, diphenhydramine hydrochloride, diphenhydramine salicylate, diphenhydramine, chlorpheniramine hydrochloride, chlorpheniramine maleate isothipendyl hydrochloride, tripelennamine hydrochloride, promethazine hydrochloride, methdilazine hydrochloride dibucaine hydrochloride, dibucaine, lidocaine hydrochloride, lidocaine, benzocaine, p-buthylaminobenzoic acid 2-(die-ethylamino) ethyl ester hydrochloride, procaine hydrochloride, tetracaine, tetracaine hydrochloride, chloroprocaine hydrochloride, oxyprocaine hydrochloride, mepivacaine, cocaine hydrochloride, piperocaine hydrochloride, dyclonine, dyclonine hydrochloride, thimerosal, phenol, thymol, benzalkonium chloride, benzethonium chloride, chlorhexidine, povidone iodide, cetylpyridinium chloride, eugenol, trimethylammonium bromide, naphazoline nitrate, tetrahydrozoline hydrochloride, oxymetazoline hydrochloride, phenylephrine hydrochloride, tramazoline hydrochloride, thrombin, phytonadione, protamine sulfate, aminocaproic acid, tranexamic acid, carbazochrome, carbaxochrome sodium sulfanate, rutin, hesperidin, sulfamine, sulfathiazole, sulfadiazine, homosulfamine, sulfisoxazole, sulfisomidine, sulfamethizole, nitrofurazone, penicillin, meticillin, oxacillin, cefalotin, cefalordin, erythromcycin, lincomycin, tetracycline, chlortetracycline, oxytetracycline, metacycline, chloramphenicol, kanamycin, streptomycin, gentamicin, bacitracin, cycloserine, salicylic acid, podophyllum resin, podolifox, cantharidin, chloroacetic acids, silver nitrate, protease inhibitors, thymadine kinase inhibitors, sugar or glycoprotein synthesis inhibitors, structural protein synthesis inhibitors, attachment and adsorption inhibitors, and nucleoside analogues such as acyclovir, penciclovir, valacyclovir, and ganciclovir, heparin, insulin, LHRH, TRH, interferons, oligonuclides, calcitonin, octreotide, omeprazone, fluoxetine, ethinylestradiol, amiodipine, paroxetine, enalapril, lisinopril, leuprolide, prevastatin, lovastatin, norethindrone, risperidone, olanzapine, albuterol, hydrochlorothiazide, pseudoephridrine, warfarin, terazosin, cisapride, ipratropium, busprione, methylphenidate, levothyroxine, zolpidem, levonorgestrel, glyburide, benazepril, medroxyprogesterone, clonazepam, ondansetron, losartan, quinapril, nitroglycerin, midazolam versed, cetirizine, doxazosin, glipizide, vaccine hepatitis B, salmeterol, sumatriptan, triamcinolone acetonide, goserelin, beclomethasone, granisteron, desogestrel, alprazolam, estradiol, nicotine, interferon beta 1A, cromolyn, fosinopril, digoxin, fluticasone, bisoprolol, calcitril, captorpril, butorphanol, clonidine, premarin, testosterone, sumatriptan, clotrimazole, bisacodyl, dextromethorphan, nitroglycerine, nafarelin, dinoprostone, nicotine, bisacodyl, goserelin, and granisetron. In certain embodiments, the pharmaceutically active component can be epinephrine, a prodrug of epinephrine a benzodiazepine such as diazepam or lorazepam or alprazolam.

### Epinephrine/Dipivefrin Examples

In one example, a composition including epinephrine or its salts or esters (such as dipivefrin) can have a biodelivery profile similar to that of epinephrine administered by injection, for example, using an EpiPen. Epinephrine or its prodrug can be present in an amount of from about 0.01 mg to about 100 mg per dosage, for example, at a 0.1 mg, 5 mg, 10 mg, 20 mg, 30 mg, 40 mg, 50 mg, 60 mg, 70 mg, 80 mg, 90 mg or 100 mg dosage, including greater than 0.1 mg, more than 5 mg, more than 20 mg, more than 30 mg, more than 40 mg, more than 50 mg, more than 60 mg, more than 70 mg, more than 80 mg, more than 90 mg, or less than 100 mg, less than 90 mg, less than 80 mg, less than 70 mg, less than 60 mg, less than 50 mg, less than 40 mg, less than 30 mg, less than 20 mg, less than 10 mg, or less than 5 mg, or any combination thereof. In another example, a composition including diazepam can have a biodelivery profile similar to that of a diazepam tablet or gel, or better.

Dipivefrin can be present in an amount of from about 0.5 mg to about 100 mg per dosage, for example, at a 0.5 mg, 1 mg, 5 mg, 10 mg, 20 mg, 30 mg, 40 mg, 50 mg, 60 mg, 70 mg, 80 mg, 90 mg or 100 mg dosage including greater than 1 mg, more than 5 mg, more than 20 mg, more than 30 mg, more than 40 mg, more than 50 mg, more than 60 mg, more than 70 mg, more than 80 mg, more than 90 mg, or less than 100 mg, less than 90 mg, less than 80 mg, less than 70 mg, less than 60 mg, less than 50 mg, less than 40 mg, less than 30 mg, less than 20 mg, less than 10 mg, or less than 5 mg, or any combination thereof.

In another example, a composition (e.g., including epinephrine) can have a suitable nontoxic, nonionic alkyl glycoside having a hydrophobic alkyl group joined by a linkage to a hydrophilic saccharide in combination with a mucosal delivery-enhancing agent selected from: (a) an aggregation inhibitory agent; (b) a charge-modifying agent; (c) a pH control agent; (d) a degradative enzyme inhibitory agent; (e) a mucolytic or mucus clearing agent; (f) a ciliostatic agent; (g) a membrane penetration-enhancing agent selected from: (i) a surfactant; (ii) a bile salt; (ii) a phospholipid additive, mixed micelle, liposome, or carrier; (iii) an alcohol; (iv) an enamine; (v) an NO donor compound; (vi) a long chain amphipathic molecule; (vii) a hydrophobic penetration enhancer; (viii) sodium or a salicylic acid derivative; (ix) a glycerol ester of acetoacetic acid; (x) a cyclodextrin or beta-cyclodextrin derivative; (xi) a medium-chain fatty acid; (xii) a chelating agent; (xiii) an amino acid or salt thereof, (xiv) an N-acetylamino acid or salt thereof, (xv) an enzyme degradative to a selected membrane component; (ix) an inhibitor of fatty acid synthesis; (x) an inhibitor of cholesterol synthesis; and (xi) any combination of the membrane penetration enhancing agents recited in (i)-(x); (h) a modulatory agent of epithelial junction physiology; (i) a vasodilator agent; (j) a selective transport-enhancing agent; or (k) a stabilizing delivery vehicle, carrier, mucoadhesive, support or complex-forming species with which the compound is effectively combined, associated, contained, encapsulated or bound resulting in stabilization of the compound for enhanced mucosal delivery, wherein the formulation of the compound with the transmucosal delivery-enhancing agents provides for increased bioavailability of the compound in a blood plasma of a subject. The formulation can include approximately the same active pharmaceutical ingredient (API): enhancer ratio as in the other examples for epinephrine.

Administering epinephrine as a prodrug such as dipivefrin confers certain advantages. For one, dipivefrin is lipophilic and therefore has a higher permeation through a mucosa. It also has a longer plasma half life due to higher protein binding. It is capable of sustained blood levels, and does not interact with α-receptors, therefore minimizing or eliminating unwanted or harmful vasoconstriction.

A film and/or its components can be water-soluble, water swellable or water-insoluble. The term “water-soluble” can refer to substances that are at least partially dissolvable in an aqueous solvent, including but not limited to water. The term “water-soluble” may not necessarily mean that the substance is 100% dissolvable in the aqueous solvent. The term “water-insoluble” refers to substances that are not dissolvable in an aqueous solvent, including but not limited to water. A solvent can include water, or alternatively can include other solvents (preferably, polar solvents) by themselves or in combination with water.

The composition can include a polymeric matrix. Any desired polymeric matrix may be used, provided that it is orally dissolvable or erodible. The dosage should have enough bioadhesion to not be easily removed and it should form a gel like structure when administered. They can be moderate-dissolving in the oral cavity and particularly suitable for delivery of pharmaceutically active components, although both fast release, delayed release, controlled release and sustained release compositions are also among the various embodiments contemplated.

### Branched Polymers

The pharmaceutical composition film can include dendritic polymers which can include highly branched macromolecules with various structural architectures. The dendritic polymers can include dendrimers, dendronised polymers (dendrigrafted polymers), linear dendritic hybrids, multi-arm star polymers, or hyperbranched polymers.

Hyperbranched polymers are highly branched polymers with imperfections in their structure. However they can be synthesized in a single step reaction which can be an advantage over other dendritic structures and are therefore suitable for bulk volume applications. The properties of these polymers apart from their globular structure are the abundant functional groups, intramolecular cavities, low viscosity and high solubility. Dendritic polymers have been used in several drug delivery applications. See, e.g., Dendrimers as Drug Carriers: Applications in Different Routes of Drug Administration. J Pharm Sci, VOL. 97, 2008, 123-143, which is incorporated by reference herein.

The dendritic polymers can have internal cavities which can encapsulate drugs. The steric hindrance caused by the highly dense polymer chains might prevent the crystallization of the drugs. Thus, branched polymers can provide additional advantages in formulating crystallizable drugs in a polymer matrix.

Examples of suitable dendritic polymers include poly(ether) based dendrons, dendrimers and hyperbranched polymers, poly(ester) based dendrons, dendrimers and hyperbranched polymers, poly(thioether) based dendrons, dendrimers and hyperbranched polymers, poly(amino acid) based dendrons dendrimers and hyperbranched polymers, poly(arylalkylene ether) based dendrons, dendrimers and hyperbranched polymers, poly(alkyleneimine) based dendrons, dendrimers and hyperbranched polymers, poly(amidoamine) based dendrons, dendrimers or hyperbranched polymers.

Other examples of hyperbranched polymers include poly(amines)s, polycarbonates, poly(ether ketone)s, polyurethanes, polycarbosilanes, polysiloxanes, poly(ester amine)s, poly(sulfone amine)s, poly(urea urethane)s and polyether polyols such as polyglycerols.

A film can be produced by a combination of at least one polymer and a solvent, optionally including other components. The solvent may be water, a polar organic solvent including, but not limited to, ethanol, isopropanol, acetone, or any combination thereof. In some embodiments, the solvent may be a non-polar organic solvent, such as methylene chloride. The film may be prepared by utilizing a selected casting or deposition method and a controlled drying process. For example, the film may be prepared through a controlled drying processes, which include application of heat and/or radiation energy to the wet film matrix to form a visco-elastic structure, thereby controlling the uniformity of content of the film. The controlled drying processes can include air alone, heat alone or heat and air together contacting the top of the film or bottom of the film or the substrate supporting the cast or deposited or extruded film or contacting more than one surface at the same time or at different times during the drying process. Some of such processes are described in more detail in U.S. Pat. Nos. 8,765,167 and 8,652,378, each of which are incorporated by reference herein. Alternatively, the films may be extruded as described in U.S. Patent Publication No. 2005/0037055 A1, which is incorporated by reference herein.

A polymer included in the films may be water-soluble, water-swellable, water-insoluble, or a combination of one or more either water-soluble, water-swellable or water-insoluble polymers. The polymer may include cellulose, cellulose derivatives or gums. Specific examples of useful water-soluble polymers include, but are not limited to, polyethylene oxide, pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, xanthan gum, tragancanth gum, guar gum, acacia gum, arabic gum, polyacrylic acid, methylmethacrylate copolymer, carboxyvinyl copolymers, starch, gelatin, and combinations thereof. Specific examples of useful water-insoluble polymers include, but are not limited to, ethyl cellulose, hydroxypropyl ethyl cellulose, cellulose acetate phthalate, hydroxypropyl methyl cellulose phthalate and combinations thereof. For higher dosages, it may be desirable to incorporate a polymer that provides a high level of viscosity as compared to lower dosages.

As used herein the phrase “water-soluble polymer” and variants thereof refer to a polymer that is at least partially soluble in water, and desirably fully or predominantly soluble in water, or absorbs water. Polymers that absorb water are often referred to as being water-swellable polymers. The materials useful with the present invention may be water-soluble or water-swellable at room temperature and other temperatures, such as temperatures exceeding room temperature. Moreover, the materials may be water-soluble or water-swellable at pressures less than atmospheric pressure. In some embodiments, films formed from such water-soluble polymers may be sufficiently water-soluble to be dissolvable upon contact with bodily fluids.

Other polymers useful for incorporation into the films include biodegradable polymers, copolymers, block polymers or combinations thereof. It is understood that the term “biodegradable” is intended to include materials that chemically degrade, as opposed to materials that physically break apart (i.e., bioerodable materials). The polymers incorporated in the films can also include a combination of biodegradable or bioerodable materials. Among the known useful polymers or polymer classes which meet the above criteria are: poly(glycolic acid) (PGA), poly(lactic acid) (PLA), polydioxanes, polyoxalates, poly(alpha-esters), polyanhydrides, polyacetates, polycaprolactones, poly(orthoesters), polyamino acids, polyaminocarbonates, polyurethanes, polycarbonates, polyamides, poly(alkyl cyanoacrylates), and mixtures and copolymers thereof. Additional useful polymers include, stereopolymers of L- and D-lactic acid, copolymers of bis(p-carboxyphenoxy)propane acid and sebacic acid, sebacic acid copolymers, copolymers of caprolactone, poly(lactic acid)/poly(glycolic acid)/polyethyleneglycol copolymers, copolymers of polyurethane and (poly(lactic acid), copolymers of alpha-amino acids, copolymers of alpha-amino acids and caproic acid, copolymers of alpha-benzyl glutamate and polyethylene glycol, copolymers of succinate and poly(glycols), polyphosphazene, polyhydroxy-alkanoates or mixtures thereof. The polymer matrix can include one, two, three, four or more components.

Although a variety of different polymers may be used, it is desired to select polymers that provide mucoadhesive properties to the film, as well as a desired dissolution and/or disintegration rate. In particular, the time period for which it is desired to maintain the film in contact with the mucosal tissue depends on the type of pharmaceutically active component contained in the composition. Some pharmaceutically active components may only require a few minutes for delivery through the mucosal tissue, whereas other pharmaceutically active components may require up to several hours or even longer. Accordingly, in some embodiments, one or more water-soluble polymers, as described above, may be used to form the film. In other embodiments, however, it may be desirable to use combinations of water-soluble polymers and polymers that are water-swellable, water-insoluble and/or biodegradable, as provided above. The inclusion of one or more polymers that are water-swellable, water-insoluble and/or biodegradable may provide films with slower dissolution or disintegration rates than films formed from water-soluble polymers alone. As such, the film may adhere to the mucosal tissue for longer periods of time, such as up to several hours, which may be desirable for delivery of certain pharmaceutically active components.

Desirably, an individual film dosage of the pharmaceutical film can have a suitable thickness, and small size, which is between about 0.0625-3 inch by about 0.0625-3 inch. The film size can also be greater than 0.0625 inch, greater than 0.5 inch, greater than 1 inch, greater than 2 inches, about 3 inches, and greater than 3 inches, less than 3 inches, less than 2 inches, less than 1 inch, less than 0.5 inch, less than 0.0625 inch in at least one aspect, or greater than 0.0625 inch, greater than 0.5 inch, greater than 1 inch, greater than 2 inches, or greater than 3 inches, about 3 inches, less than 3 inches, less than 2 inches, less than 1 inch, less than 0.5 inch, less than 0.0625 inch in another aspect. The aspect ratio, including thickness, length, and width can be optimized by a person of ordinary skill in the art based on the chemical and physical properties of the polymeric matrix, the active pharmaceutical ingredient, dosage, enhancer, and other additives involved as well as the dimensions of the desired dispensing unit. The film dosage should have good adhesion when placed in the buccal cavity or in the sublingual region of the user. Further, the film dosage should disperse and dissolve at a moderate rate, most desirably dispersing within about 1 minute and dissolving within about 3 minutes. In some embodiments, the film dosage may be capable of dispersing and dissolving at a rate of between about 1 to about 30 minutes, for example, about 1 to about 20 minutes, or more than 1 minute, more than 5 minutes, more than 7 minutes, more than 10 minutes, more than 12 minutes, more than 15 minutes, more than 20 minutes, more than 30 minutes, about 30 minutes, or less than 30 minutes, less than 20 minutes, less than 15 minutes, less than 12 minutes, less than 10 minutes, less than 7 minutes, less than 5 minutes, or less than 1 minute. Sublingual dispersion rates may be shorter than buccal dispersion rates.

For instance, in some embodiments, the films may include polyethylene oxide alone or in combination with a second polymer component. The second polymer may be another water-soluble polymer, a water-swellable polymer, a water-insoluble polymer, a biodegradable polymer or any combination thereof. Suitable water-soluble polymers include, without limitation, any of those provided above. In some embodiments, the water-soluble polymer may include hydrophilic cellulosic polymers, such as hydroxypropyl cellulose and/or hydroxypropylmethyl cellulose. In some embodiments, one or more water-swellable, water-insoluble and/or biodegradable polymers also may be included in the polyethylene oxide-based film. Any of the water-swellable, water-insoluble or biodegradable polymers provided above may be employed. The second polymer component may be employed in amounts of about 0% to about 80% by weight in the polymer component, more specifically about 30% to about 70% by weight, and even more specifically about 40% to about 60% by weight, including greater than 5%, greater than 10%, greater than 15%, greater than 20%, greater than 30%, greater than 40%, greater than 50%, greater than 60%, and greater than 70%, about 70%, less than 70%, less than 60%, less than 50%, less than 40%, less than 30%, less than 20%, less than 10% or less than 5% by weight.

Additives may be included in the films. Examples of classes of additives include preservatives, antimicrobials, excipients, lubricants, buffering agents, stabilizers, blowing agents, pigments, coloring agents, fillers, bulking agents, sweetening agents, flavoring agents, fragrances, release modifiers, adjuvants, plasticizers, desiccants, flow accelerators, mold release agents, polyols, granulating agents, diluents, binders, buffers, absorbents, glidants, adhesives, anti-adherents, acidulants, softeners, resins, demulcents, solvents, surfactants, emulsifiers, elastomers, anti-tacking agents, anti-static agents and mixtures thereof. These additives may be added with the pharmaceutically active component(s). As used herein, the term “stabilizer” means an excipient capable of preventing aggregation or other physical degradation, as well as chemical degradation, of the active pharmaceutical ingredient, another excipient, or the combination thereof.

Stabilizers may also be classified as plasticizers, desiccants, antioxidants, sequestrants, pH modifiers, emulsifiers and/or surfactants, and UV stabilizers.

An absorbant component, adsorbant component, or desiccant is a compound that is designed to safeguard pharmaceutical products from liquid damage, for example, water or other liquids, by absorbing moisture by physical adsorption or by chemical reaction, thereby improving the stability of the product. A suitable the absorbant component, adsorbant component, or desiccant can include a silica, fumed silica or a mesoporous silica. The absorbant component, adsorbant component, or desiccant can be an amorphouse silicon dioxide. Examples of the absorbant component, adsorbant component, or desiccant include silicon dioxide variants such as a Cab-o-Sil or a Syloid product. The desiccant can be 1-15% by weight of the pharmaceutical composition. For example, the absorbant component, adsorbant component, or desiccant can be 2%, 3%, 4%, 5%, 6%, 7%, 8%, 9%, 10%, 11%, 12%, 13%, 14%, or 15% w/w of the pharmaceutical composition. The silicon dioxide can have an average surface area of about 25 m²/g, 50 m²/g, 100 m²/g, 200 m²/g, 300 m²/g, 400 m²/g, 500 m²/g, or 600 m²/g. The silicon dioxide can have an average particle size of about 1 micron, 2 microns, 4 microns, 5 microns, 8 microns, about 10 microns or greater. In certain embodiments, one or more oil components of the composition can be adsorbed on the desiccant. The absorbant component, adsorbant component, or desiccant can adsorb up to about 0.5 mL, 1.0 mL, 1.5 mL, or 2.0 mL of liquid per gram of desiccant.

Antioxidants (i.e., pharmaceutically compatible compound(s) or composition(s) that decelerates, inhibits, interrupts and/or stops oxidation processes) include, in particular, the following substances: tocopherols and the esters thereof, sesamol of sesame oil, coniferyl benzoate of benzoin resin, nordihydroguaietic resin and nordihydroguaiaretic acid (NDGA), gallates (among others, methyl, ethyl, propyl, amyl, butyl, lauryl gallates), butylated hydroxyanisole (BHA/BHT, also butyl-p-cresol); ascorbic acid and salts and esters thereof (for example, acorbyl palmitate), erythorbinic acid (isoascorbinic acid) and salts and esters thereof, monothioglycerol, sodium formaldehyde sulfoxylate, sodium metabisulfite, sodium bisulfite, sodium sulfite, potassium metabisulfite, butylated hydroxyanisole, butylated hydroxytoluene (BHT), propionic acid. Typical antioxidants are tocopherol such as, for example, α-tocopherol and the esters thereof, butylated hydroxytoluene and butylated hydroxyanisole. The terms “tocopherol” also includes esters of tocopherol. A known tocopherol is α-tocopherol. The term “α-tocopherol” includes esters of α-tocopherol (for example, α-tocopherol acetate).

Sequestrants (i.e., any compounds which can engage in host-guest complex formation with another compound, such as the active ingredient or another excipient; also referred to as a sequestering agent) include calcium chloride, calcium disodium ethylene diamine tetra-acetate, glucono delta-lactone, sodium gluconate, potassium gluconate, sodium tripolyphosphate, sodium hexametaphosphate, and combinations thereof. Sequestrants also include cyclic oligosaccharides, such as cyclodextrins, cyclomannins (5 or more α-D-mannopyranose units linked at the 1,4 positions by a linkages), cyclogalactins (5 or more β-D-galactopyranose units linked at the 1,4 positions by R linkages), cycloaltrins (5 or more α-D-altropyranose units linked at the 1,4 positions by a linkages), and combinations thereof.

pH modifiers include acids (e.g., tartaric acid, citric acid, lactic acid, fumaric acid, phosphoric acid, ascorbic acid, acetic acid, succininc acid, adipic acid and maleic acid), acidic amino acids (e.g., glutamic acid, aspartic acid, etc.), inorganic salts (alkali metal salt, alkaline earth metal salt, ammonium salt, etc.) of such acidic substances, a salt of such acidic substance with an organic base (e.g., basic amino acid such as lysine, arginine and the like, meglumine and the like), and a solvate (e.g., hydrate) thereof. Other examples of pH modifiers include silicified microcrystalline cellulose, magnesium aluminometasilicate, calcium salts of phosphoric acid (e.g., calcium hydrogen phosphate anhydrous or hydrate, calcium, sodium or potassium carbonate or hydrogencarbonate and calcium lactate or mixtures thereof), sodium and/or calcium salts of carboxymethyl cellulose, cross-linked carboxymethylcellulose (e.g., croscarmellose sodium and/or calcium), polacrilin potassium, sodium and or/calcium alginate, docusate sodium, magnesium calcium, aluminium or zinc stearate, magnesium palmitate and magnesium oleate, sodium stearyl fumarate, and combinations thereof.

Examples of emulsifiers and/or surfactants include poloxamers or pluronics, polyethylene glycols, polyethylene glycol monostearate, polysorbates, sodium lauryl sulfate, polyethoxylated and hydrogenated castor oil, alkyl polyoside, a grafted water soluble protein on a hydrophobic backbone, lecithin, glyceryl monostearate, glyceryl monostearate/polyoxyethylene stearate, ketostearyl alcohol/sodium lauryl sulfate, carbomer, phospholipids, (C₁₀-C₂₀)-alkyl and alkylene carboxylates, alkyl ether carboxylates, fatty alcohol sulfates, fatty alcohol ether sulfates, alkylamide sulfates and sulfonates, fatty acid alkylamide polyglycol ether sulfates, alkanesulfonates and hydroxyalkanesulfonates, olefinsulfonates, acyl esters of isethionates, α-sulfo fatty acid esters, alkylbenzenesulfonates, alkylphenol glycol ether sulfonates, sulfosuccinates, sulfosuccinic monoesters and diesters, fatty alcohol ether phosphates, protein/fatty acid condensation products, alkyl monoglyceride sulfates and sulfonates, alkylglyceride ether sulfonates, fatty acid methyltaurides, fatty acid sarcosinates, sulforicinoleates, and acylglutamates, quaternary ammonium salts (e.g., di-(C₁₀-C₂₄)-alkyl-dimethylammonium chloride or bromide), (C₁₀-C₂₄)-alkyl-dimethylethylammonium chloride or bromide, (C₁₀-C₂₄)-alkyl-trimethylammonium chloride or bromide (e.g., cetyltrimethylammonium chloride or bromide), (C₁₀-C₂₄)-alkyl-dimethylbenzylammonium chloride or bromide (e.g., (C₁₂-C₁₈)-alkyl-dimethylbenzylammonium chloride), N—(C₁₀-C₁₈)-alkyl-pyridinium chloride or bromide (e.g., N—(C₁₂-C₁₆)-alkyl-pyridinium chloride or bromide), N—(C₁₀-C₁₈)-alkyl-isoquinolinium chloride, bromide or monoalkyl sulfate, N—(C₁₂-C₁₈)-alkyl-polyoylaminoformylmethylpyridinium chloride, N—(C₁₂-C₁₈)-alkyl-N-methylmorpholinium chloride, bromide or monoalkyl sulfate, N—(C₁₂-C₁₈)-alkyl-N-ethylmorpholinium chloride, bromide or monoalkyl sulfate, (C₁₆-C₁₅)-alkyl-pentaoxethylammonium chloride, diisobutylphenoxyethoxyethyldimethylbenzylammonium chloride, salts of N,N-di-ethylaminoethylstearylamide and -oleylamide with hydrochloric acid, acetic acid, lactic acid, citric acid, phosphoric acid, N-acylaminoethyl-N,N-diethyl-N-methylammonium chloride, bromide or monoalkyl sulfate, and N-acylaminoethyl-N,N-diethyl-N-benzylammonium chloride, bromide or monoalkyl sulfate (in the foregoing, “acyl” standing for, e.g., stearyl or oleyl), and combinations thereof.

Examples of UV stabilizers include UV absorbers (e.g., benzophenones), UV quenchers (i.e., any compound that dissipates UV energy as heat, rather than allowing the energy to have a degradation effect), scavengers (i.e., any compound that eliminates free radicals resulting from exposure to UV radiation), and combinations thereof.

In other embodiments, stabilizers include ascorbyl palmitate, ascorbic acid, alpha tocopherol, butylated hydroxytoluene, buthylated hydroxyanisole, cysteine HCl, citric acid, ethylenediamine tetra acetic acid (EDTA), methionine, sodium citrate, sodium ascorbate, sodium thiosulfate, sodium metabi sulfite, sodium bisulfite, propyl gallate, glutathione, thioglycerol, singlet oxygen quenchers, hydroxyl radical scavengers, hydroperoxide removing agents, reducing agents, metal chelators, detergents, chaotropes, and combinations thereof. “Singlet oxygen quenchers” include, but are not limited to, alkyl imidazoles (e.g., histidine, L-camosine, histamine, imidazole 4-acetic acid), indoles (e.g., tryptophan and derivatives thereof, such as N-acetyl-5-methoxytryptamine, N-acetylserotonin, 6-methoxy-1,2,3,4-tetrahydro-beta-carboline), sulfur-containing amino acids (e.g., methionine, ethionine, djenkolic acid, lanthionine, N-formyl methionine, felinine, S-allyl cysteine, S-aminoethyl-L-cysteine), phenolic compounds (e.g., tyrosine and derivatives thereof), aromatic acids (e.g., ascorbate, salicylic acid, and derivatives thereof), azide (e.g., sodium azide), tocopherol and related vitamin E derivatives, and carotene and related vitamin A derivatives. “Hydroxyl radical scavengers” include, but are not limited to azide, dimethyl sulfoxide, histidine, mannitol, sucrose, glucose, salicylate, and L-cysteine. “Hydroperoxide removing agents” include, but are not limited to catalase, pyruvate, glutathione, and glutathione peroxidases. “Reducing agents” include, but are not limited to, cysteine and mercaptoethylene. “Metal chelators” include, but are not limited to, EDTA, EGTA, ω-phenanthroline, and citrate. “Detergents” include, but are not limited to, SDS and sodium lauroyl sarcosyl. “Chaotropes” include, but are not limited to guandinium hydrochloride, isothiocyanate, urea, and formamide. As discussed herein, stabilizers can be present in 0.0001%-50% by weight, including greater than 0.0001%, greater than 0.001%, greater than 0.01%, greater than 0.1%, greater than 1%, greater than 5%, greater than 10%, greater than 20%, greater than 30%, greater than 40%, greater than 50%, less than 50%, less than 40%, less than 30%, less than 20%, less than 10%, less than 1%, less than 0.1%, less than 0.01%, less than 0.001%, or less than 0.0001% by weight.

Useful additives can include, for example, gelatin, vegetable proteins such as sunflower protein, soybean proteins, cotton seed proteins, peanut proteins, grape seed proteins, whey proteins, whey protein isolates, blood proteins, egg proteins, acrylated proteins, water-soluble polysaccharides such as alginates, carrageenans, guar gum, agar-agar, xanthan gum, gellan gum, gum arabic and related gums (gum ghatti, gum karaya, gum tragancanth), pectin, water-soluble derivatives of cellulose: alkylcelluloses hydroxyalkylcelluloses and hydroxyalkylalkylcelluloses, such as methylcellulose, hydroxymethylcellulose, hydroxyethylcellulose, hydroxypropylcellulose, hydroxyethylmethylcellulose, hydroxypropylmethylcellulose, hydroxybutylmethylcellulose, cellulose esters and hydroxyalkylcellulose esters such as cellulose acetate phthalate (CAP), hydroxypropylmethylcellulose (HPMC); carboxyalkylcelluloses, carboxyalkylalkylcelluloses, carboxyalkylcellulose esters such as carboxymethylcellulose and their alkali metal salts; water-soluble synthetic polymers such as polyacrylic acids and polyacrylic acid esters, polymethacrylic acids and polymethacrylic acid esters, polyvinylacetates, polyvinylalcohols, polyvinylacetatephthalates (PVAP), polyvinylpyrrolidone (PVP), PVA/vinyl acetate copolymer, pea starch, pregelatinized hydroxypropyl pea starch, and polycrotonic acids; also suitable are phthalated gelatin, gelatin succinate, crosslinked gelatin, shellac, water-soluble chemical derivatives of starch, cationically modified acrylates and methacrylates possessing, for example, a tertiary or quaternary amino group, such as the diethylaminoethyl group, which may be quaternized if desired; or other similar polymers.

The additional components can range up to about 80%, desirably about 0.005% to 50% and more desirably within the range of 1% to 20% based on the weight of all composition components, including greater than 1%, greater than 5%, greater than 10%, greater than 20%, greater than 30%, greater than 40%, greater than 50%, greater than 60%, greater than 70%, about 80%, greater than 80%, less than 80%, less than 70%, less than 60%, less than 50%, less than 40%, less than 30%, less than 20%, less than 10%, less than 5%, about 3%, or less than 1%. Other additives can include anti-tacking, flow agents and opacifiers, such as the oxides of magnesium aluminum, silicon, titanium, etc. desirably in a concentration range of about 0.005% to about 5% by weight and desirably about 0.02% to about 2% based on the weight of all film components, including greater than 0.02%, greater than 0.2%, greater than 0.5%, greater than 1%, greater than 1.5%, greater than 2%, greater than 4%, about 5%, greater than 5%, less than 4%, less than 2%, less than 1%, less than 0.5%, less than 0.2%, or less than 0.02%.

In certain embodiments, the composition can include plasticizers, which can include polyalkylene oxides, such as polyethylene glycols, polypropylene glycols, polyethylene-propylene glycols, organic plasticizers with low molecular weights, such as glycerol, glycerol monoacetate, diacetate or triacetate, triacetin, polysorbate, cetyl alcohol, propylene glycol, sugar alcohols sorbitol, sodium diethylsulfosuccinate, triethyl citrate, tributyl citrate, phytoextracts, fatty acid esters, fatty acids, oils and the like, added in concentrations ranging from about 0.1% to about 40%, and desirably ranging from about 0.5% to about 20% based on the weight of the composition including greater than 0.5%, greater than 1%, greater than 1.5%, greater than 2%, greater than 4%, greater than 5%, greater than 10%, greater than 15%, about 20%, greater than 20%, less than 20%, less than 15%, less than 10%, less than 5%, less than 4%, less than 2%, less than 1%, and less than 0.5%. There may further be added compounds to improve the texture properties of the film material such as animal or vegetable fats, desirably in their hydrogenated form. The composition can also include compounds to improve the textural properties of the product. Other ingredients can include binders which contribute to the ease of formation and general quality of the films. Non-limiting examples of binders include starches, natural gums, pregelatinized starches, gelatin, polyvinylpyrrolidone, methylcellulose, sodium carboxymethylcellulose, ethylcellulose, polyacrylamides, polyvinyloxoazolidone, or polyvinylalcohols.

Further potential additives include solubility enhancing agents, such as substances that form inclusion compounds with active components. Such agents may be useful in improving the properties of very insoluble and/or unstable actives. In general, these substances are doughnut-shaped molecules with hydrophobic internal cavities and hydrophilic exteriors. Insoluble and/or instable pharmaceutically active components may fit within the hydrophobic cavity, thereby producing an inclusion complex, which is soluble in water. Accordingly, the formation of the inclusion complex permits very insoluble and/or unstable pharmaceutically active components to be dissolved in water. A particularly desirable example of such agents are cyclodextrins, which are cyclic carbohydrates derived from starch. Other similar substances, however, are considered well within the scope of the present invention.

Suitable coloring agents include food, drug and cosmetic colors (FD&C), drug and cosmetic colors (D&C), or external drug and cosmetic colors (Ext. D&C). These colors are dyes, their corresponding lakes, and certain natural and derived colorants. Lakes are dyes absorbed on aluminum hydroxide. Other examples of coloring agents include known azo dyes, organic or inorganic pigments, or coloring agents of natural origin. Inorganic pigments are preferred, such as the oxides or iron or titanium, these oxides, being added in concentrations ranging from about 0.001 to about 10%, and preferably about 0.5 to about 3%, including greater than 0.001%, greater than 0.01%, greater than 0.1%, greater than 0.5%, greater than 1%, greater than 2%, greater than 5%, about 10%, greater than 10%, less than 10%, less than 5%, less than 2%, less than 1%, less than 0.5%, less than 0.1%, less than 0.01%, or less than 0.001%, based on the weight of all the components.

Flavors may be chosen from natural and synthetic flavoring liquids. An illustrative list of such agents includes volatile oils, synthetic flavor oils, flavoring aromatics, oils, liquids, oleoresins or extracts derived from plants, leaves, flowers, fruits, stems and combinations thereof. A non-limiting representative list of examples includes mint oils, cocoa, and citrus oils such as lemon, orange, lime and grapefruit and fruit essences including apple, pear, peach, grape, strawberry, raspberry, cherry, plum, pineapple, apricot or other fruit flavors. Other useful flavorings include aldehydes and esters such as benzaldehyde (cherry, almond), citral i.e., alphacitral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanol (green fruit), and 2-dodecenal (citrus, mandarin), combinations thereof and the like.

The sweeteners may be chosen from the following non-limiting list: glucose (corn syrup), dextrose, invert sugar, fructose, and combinations thereof, saccharin and its various salts such as the sodium salt; dipeptide based sweeteners such as aspartame, neotame, advantame; dihydrochalcone compounds, glycyrrhizin; Stevia Rebaudiana (Stevioside); chloro derivatives of sucrose such as sucralose; sugar alcohols such as sorbitol, mannitol, xylitol, and the like. Also contemplated are hydrogenated starch hydrolysates and the synthetic sweetener 3,6-dihydro-6-methyl-1-1-1,2,3-oxathiazin-4-one-2,2-dioxide, particularly the potassium salt (acesulfame-K), and sodium and calcium salts thereof, and natural intensive sweeteners, such as Lo Han Kuo. Other sweeteners may also be used.

Anti-foaming and/or de-foaming components may also be used with the films. These components aid in the removal of air, such as entrapped air, from the film-forming compositions. Such entrapped air may lead to non-uniform films. Simethicone is one particularly useful anti-foaming and/or de-foaming agent. The present invention, however, is not so limited and other suitable anti-foam and/or de-foaming agents may be used. Simethicone and related agents may be employed for densification purposes. More specifically, such agents may facilitate the removal of voids, air, moisture, and similar undesired components, thereby providing denser and thus more uniform films. Agents or components which perform this function can be referred to as densification or densifying agents. As described above, entrapped air or undesired components may lead to non-uniform films.

Any other optional components described in commonly assigned U.S. Pat. Nos. 7,425,292 and 8,765,167, referred to above, also may be included in the films described herein.

The film compositions further desirably contains a buffer so as to control the pH of the film composition. Any desired level of buffer may be incorporated into the film composition so as to provide the desired pH level encountered as the pharmaceutically active component is released from the composition. The buffer is preferably provided in an amount sufficient to control the release from the film and/or the absorption into the body of the pharmaceutically active component. In some embodiments, the buffer may include sodium citrate, citric acid, bitartrate salt and combinations thereof.

The pharmaceutical films described herein may be formed via any desired process. Suitable processes are set forth in U.S. Pat. Nos. 8,652,378, 7,425,292 and 7,357,891, each of which are incorporated by reference herein. In one embodiment, the film dosage composition is formed by first preparing a wet composition, the wet composition including a polymeric carrier matrix and a therapeutically effective amount of a pharmaceutically active component. The wet composition is cast into a film and then sufficiently dried to form a self-supporting film composition. The wet composition may be cast into individual dosages, or it may be cast into a sheet, where the sheet is then cut into individual dosages.

The pharmaceutical composition can adhere to a mucosal surface. The present invention finds particular use in the localized treatment of body tissues, diseases, or wounds which may have moist surfaces and which are susceptible to bodily fluids, such as the mouth, the vagina, organs, or other types of mucosal surfaces. The composition carries a pharmaceutical, and upon application and adherence to the mucosal surface, offers a layer of protection and delivers the pharmaceutical to the treatment site, the surrounding tissues, and other bodily fluids. The composition provides an appropriate residence time for effective drug delivery at the treatment site, given the control of erosion in aqueous solution or bodily fluids such as saliva, and the slow, natural erosion of the film concomitant or subsequent to the delivery.

The residence time of the composition depends on the erosion rate of the water erodable polymers used in the formulation and their respective concentrations. The erosion rate may be adjusted, for example, by mixing together components with different solubility characteristics or chemically different polymers, such as hydroxyethyl cellulose and hydroxypropyl cellulose; by using different molecular weight grades of the same polymer, such as mixing low and medium molecular weight hydroxyethyl cellulose; by using excipients or plasticizers of various lipophilic values or water solubility characteristics (including essentially insoluble components); by using water soluble organic and inorganic salts; by using crosslinking agents such as glyoxal with polymers such as hydroxyethyl cellulose for partial crosslinking; or by post-treatment irradiation or curing, which may alter the physical state of the film, including its crystallinity or phase transition, once obtained. These strategies might be employed alone or in combination in order to modify the erosion kinetics of the film. Upon application, the pharmaceutical composition film adheres to the mucosal surface and is held in place. Water absorption softens the composition, thereby diminishing the foreign body sensation. As the composition rests on the mucosal surface, delivery of the drug occurs. Residence times may be adjusted over a wide range depending upon the desired timing of the delivery of the chosen pharmaceutical and the desired lifespan of the carrier. Generally, however, the residence time is modulated between about a few seconds to about a few days. Preferably, the residence time for most pharmaceuticals is adjusted from about 5 seconds to about 24 hours. More preferably, the residence time is adjusted from about 5 seconds to about 30 minutes. In addition to providing drug delivery, once the composition adheres to the mucosal surface, it also provides protection to the treatment site, acting as an erodable bandage. Lipophilic agents can be designed to slow down erodability to decrease disintegration and dissolution.

It is also possible to adjust the kinetics of erodability of the composition by adding excipients which are sensitive to enzymes such as amylase, very soluble in water such as water soluble organic and inorganic salts. Suitable excipients may include the sodium and potassium salts of chloride, carbonate, bicarbonate, citrate, trifluoroacetate, benzoate, phosphate, fluoride, sulfate, or tartrate. The amount added can vary depending upon how much the erosion kinetics is to be altered as well as the amount and nature of the other components in the composition.

Emulsifiers typically used in the water-based emulsions described above are, preferably, either obtained in situ if selected from the linoleic, palmitic, myristoleic, lauric, stearic, cetoleic or oleic acids and sodium or potassium hydroxide, or selected from the laurate, palmitate, stearate, or oleate esters of sorbitol and sorbitol anhydrides, polyoxyethylene derivatives including monooleate, monostearate, monopalmitate, monolaurate, fatty alcohols, alkyl phenols, allyl ethers, alkyl aryl ethers, sorbitan monostearate, sorbitan monooleate and/or sorbitan monopalmitate.

The amount of pharmaceutically active component to be used depends on the desired treatment strength and the composition of the layers, although preferably, the pharmaceutical component comprises from about 0.001% to about 99%, more preferably from about 0.003 to about 75%, and most preferably from about 0.005% to about 50% by weight of the composition, including, more than 0.005%, more than 0.05%, more than 0.5%, more than 1%, more than 5%, more than 10%, more than 15%, more than 20%, more than 30%, about 50%, more than 50%, less than 50%, less than 30%, less than 20%, less than 15%, less than 10%, less than 5%, less than 1%, less than 0.5%, less than 0.05%, or less than 0.005%. The amounts of other components may vary depending on the drug or other components but typically these components comprise no more than 50%, preferably no more than 30%, and most preferably no more than 15% by total weight of the composition.

The thickness of the film may vary, depending on the thickness of each of the layers and the number of layers. As stated above, both the thickness and amount of layers may be adjusted in order to vary the erosion kinetics. Preferably, if the composition has only two layers, the thickness ranges from 0.005 mm to 2 mm, preferably from 0.01 to 1 mm, and more preferably from 0.1 to 0.5 mm, including greater than 0.1 mm, greater than 0.2 mm, about 0.5 mm, greater than 0.5 mm, less than 0.5 mm, less than 0.2 mm, or less than 0.1 mm. The thickness of each layer may vary from 10 to 90% of the overall thickness of the layered composition, and preferably varies from 30 to 60%, including greater than 10%, greater than 20%, greater than 30%, greater than 40%, greater than 50%, greater than 70%, greater than 90%, about 90%, less than 90%, less than 70%, less than 50%, less than 40%, less than 30%, less than 20%, or less than 10%. Thus, the preferred thickness of each layer may vary from 0.01 mm to 0.9 mm, or from 0.03 to 0.5 mm.

As one skilled in the art will appreciate, when systemic delivery, e.g., transmucosal or transdermal delivery is desired, the treatment site may include any area in which the film is capable of delivery and/or maintaining a desired level of pharmaceutical in the blood, lymph, or other bodily fluid. Typically, such treatment sites include the oral, esophageal, aural, ocular, anal, nasal, and vaginal mucosal tissue, as well as, the skin. If the skin is to be employed as the treatment site, then usually larger areas of the skin wherein movement will not disrupt the adhesion of the film, such as the upper arm or thigh, are preferred.

The pharmaceutical composition can also be used as a wound dressing. By offering a physical, compatible, oxygen and moisture permeable, flexible barrier which can be washed away, the film can not only protect a wound but also deliver a pharmaceutical in order to promote healing, aseptic, scarification, to ease the pain or to improve globally the condition of the sufferer. Some of the: examples given below are well suited for an application to the skin or a wound. As one skilled in the art will appreciate, the formulation might require incorporating a specific hydrophilic/hygroscopic excipient which would help in maintaining good adhesion on dry skin over an extended period of time. Another advantage of the present invention when utilized in this manner is that if one does not wish that the film be noticeable on the skin, then no dyes or colored substances need be used. If, on the other hand, one desires that the film be noticeable, a dye or colored substance may be employed.

While the pharmaceutical composition can adhere to mucosal tissues, which are wet tissues by nature, it can also be used on other surfaces such as skin or wounds. The pharmaceutical film can adhere to the skin if prior to application the skin is wet with an aqueous-based fluid such as water, saliva, wound drainage or perspiration. The film can adhere to the skin until it erodes due to contact with water by, for example, rinsing, showering, bathing or washing. The film may also be readily removed by peeling without significant damage to tissue. A Franz diffusion cell is an in vitro skin permeation assay used in formulation development. The Franz diffusion cell apparatus (FIG. 1A) consists of two chambers separated by a membrane of, for example, animal or human tissue. The test product is applied to the membrane via the top chamber. The bottom chamber contains fluid from which samples are taken at regular intervals for analysis to determine the amount of active that has permeated the membrane. Referring to FIG. 1A, a Franz diffusion cell 100 includes a donor compound 101, a donor chamber 102, a membrane 103, sampling port 104, receptor chamber 105, stir bar 106, and a heater/circulator 107.

Referring to FIG. 1B, a pharmaceutical composition is a film 100 comprising a polymeric matrix 200, the pharmaceutically active component 300 being contained in the polymeric matrix. The film can include a permeation enhancer 400.

Referring to FIGS. 2A and 2B, the graphs show the permeation of an active material from a composition. The graph shows that for the epinephrine base—solubilized in-situ vs. the inherently soluble epinephrine bitartrate, no meaningful differences were observed. Epinephrine bitartrate was selected for further development based on ease of processing. Flux is derived as slope of the amount permeated as a function of time. Steady state flux is taken from the plateau of flux vs time curve multiplied by the volume of receiver media and normalized for permeation area.

Referring to FIG. 2A, this graph shows average amount of active material permeated vs. time, with 8.00 mg/mL epinephrine bitartrate and 4.4 mg/mL epinephrine base solubilized.

Referring to FIG. 2B, this graph shows average flux vs. time, with 8.00 mg/mL epinephrine bitartrate and 4.4 mg/mL epinephrine base solubilized.

Referring to FIG. 3, this graph shows ex-vivo permeation of epinephrine bitartrate as a function of concentration. The study compared concentrations of 4 mg/mL, 8 mg/mL, 16 mg/mL and 100 mg/mL. Results showed that increasing concentration resulted in increased permeation, and level of enhancement diminishes at higher loading.

Referring to FIG. 4, this graph shows permeation of epinephrine bitartrate as a function of solution pH. Acidic conditions explored to promote stability. The results compared epinephrine bitartrate pH 3 buffer and epinephrine bitartrate pH 5 buffer, and found that the epinephrine bitartrate pH 5 buffer was slightly favorable.

Referring to FIG. 5, this graph shows the influence of enhancers on permeation of epinephrine, indicated as amount permeated as a function of time. Multiple enhancers were screened, including Labrasol, capryol 90, Plurol Oleique, Labrafil, TDM, SGDC, Gelucire 44/14 and clove oil. Significant impact on time to onset and steady state flux was achieved, and surprisingly enhanced permeation was achieved for clove oil and Labrasol.

Referring to FIGS. 6A and 6B, these graphs show the release of epinephrine on polymer platforms and the effect of enhancers on its release, indicated as amount permeated (in pg) vs. time. FIG. 6A shows the epinephrine release from different polymer platforms. FIG. 6B shows the impact of enhancers on epinephrine release.

Referring to FIG. 7, this graph shows a pharmacokinetic model in the male Yucatan, miniature swine. The study compares a 0.3 mg Epipen, a 0.12 mg Epinephrine IV and a placebo film.

Referring to FIG. 8, this graphs shows the impact of no enhancer on the concentration profiles of a 40 mg epinephrine film vs, a 0.3 mg Epipen.

Referring to FIG. 9, this graph shows the impact of Enhancer A (Labrasol) on the concentration profiles of a 40 mg epinephrine film vs, a 0.3 mg Epipen. Referring to FIG. 10, this graph shows the impact of Enhancer L (clove oil) on the concentration profiles of two 40 mg Epinephrine films (10-1-1) and (11-1-1) vs. a 0.3 mg Epipen.

Referring to FIG. 11, This graph shows the impact of Enhancer L (clove oil) and film dimension (10-1-1 thinner bigger film and 11-1-1 thicker smaller film) on the concentration profiles of 40 mg Epinephrine films vs. a 0.3 mg Epipen.

Referring to FIG. 12, this graph shows the concentration profiles for varying doses of Epinephrine films in a constant matrix for Enhancer L (clove oil) vs. a 0.3 mg Epipen.

Referring to FIG. 13, the graph shows the concentration profiles for varying doses of Epinephrine films in a constant matrix for Enhancer L (clove oil) vs. a 0.3 mg Epipen.

Referring to FIG. 14, the graph shows the concentration profiles for varying doses of Epinephrine films in a constant matrix for Enhancer A (Labrasol) vs. a 0.3 mg Epipen.

Referring to FIG. 15, this graph shows the impact of Farnesol and Farnesol in combination with Linoleic Acid on plasma concentration profiles of 40 mg Epinephrine Films vs. a 0.3 mg Epipen.

Referring to FIG. 16, this graph shows the impact of Farnesol and Farnesol in combination with Linoleic Acid on plasma concentration profiles of 40 mg Epinephrine Films vs. a 0.3 mg Epipen.

Referring to FIG. 17, this graph shows the impact of Farnesol in combination with Linoleic Acid on plasma concentration profiles of 40 mg Epinephrine Films vs. a 0.3 mg Epipen.

Referring to FIG. 18, this graph shows the impact of Farnesol and Farnesol in combination with Linoleic Acid on plasma concentration profiles of 40 mg Epinephrine Films vs. a 0.3 mg Epipen. The following examples are provided to illustrate pharmaceutical compositions, as well as, methods of making and using, pharmaceutical compositions and devices described herein.

### EXAMPLES

### Example 1

### Permeation Enhancers—Epinephrine

Permeation enhancement was studied using a number of permeation enhancers with Epinephrine Bitartrate 16.00 mg/mL concentration. The results show flux enhancement represented in the data below. For 100% Eugenol and 100% Clove Oil, the results showed steady state flux reached significantly earlier along with an unexpectedly heightened % flux enhancement.

| Donor Solution | Average | | |
|---|---|---|---|
| (16.00 mg/mL | Steady State | | Permeability |
| Epinephrine | Flux | % Flux | Coefficient |
| Bitartrate + enhancer) | (ug/cm²*min) | enhancement | (cm/s) |
| Epinephrine Bitartrate, | 1.3173 | N/A | 1.37E−06 |
| no enhancer | | | |
| 3% Clove Oil | 8.2704 | 527.84 | 8.61E−06 |
| 3% Clove Oil Repeat | 5.3776 | 308.24 | 5.60E−06 |
| 3% Eugenol | 7.1311 | 441.35 | 7.43E−06 |
| 3% Eugenyl Acetate | 1.8945 | 43.82 | 1.97E−06 |
| 3% B-Caryophyllene | 3.5200 | 167.22 | 3.67E−06 |
| 0.3% Eugenol* | 3.9735 | 201.65 | 4.14E−06 |
| 100% Eugenol ¹ | 36.8432 | 2696.92 | 3.84E−05 |
| 0.3% Clove Oil* | 3.6806 | 179.41 | 3.83E−06 |
| 100% Clove Oil ¹ | 52.5304 | 3887.81 | 5.47E−05 |
| 3% Phenol | 4.5790 | 247.61 | 4.77E−06 |
| 3% Phenol Repeat | 4.1753 | 216.97 | 4.35E−06 |
| 3% Linoleic Acid | 2.1788 | 65.40 | 2.27E−06 |
| 50% Clove Oil | 2.5673 | 94.89 | 2.67E−06 |
| 0.3% Labrasol | 3.5221 | 167.38 | 3.67E−06 |
| 3% Vanillyl Alcohol + | 1.10243 | −16.31 | 1.15E−06 |
| 6% Ethanol | | | |
| 3% Safrole | 2.60634 | 97.86 | 2.71E−06 |
| 3% Oleic Acid | 2.06597 | 56.84 | 2.15E−06 |
| 3% Oleic Acid + | 2.73655 | 107.74 | 2.85E−06 |
| 1% PEG200 | | | |
| 3% Benzyl Alcohol | 1.38455 | 5.11 | 1.44E−06 |

¹ steady state flux reached at much earlier time point
*0.3% Eugenol vs 0.3% Clove -similar flux rates to one another

For these examples, clove oil was obtained from clove leaf. Similar results may be obtained from clove oil from clove bud and/or clove stem. Based on this data, similar permeability enhancement results can be expected from pharmaceutical compounds structurally similar to epinephrine.

### Example 2

### General Permeation Procedure—Ex Vivo Permeation Study Protocol

In one example, a permeation procedure is conducted as follows. A temperature bath is set to 37° C., and receiver media is placed in a water bath to adjust the temperature and begin degassing. A franz diffusion cell is obtained and prepared. The franz diffusion cell includes a donor compound, a donor chamber, a membrane, sampling port, receptor chamber, stir bar, and a heater/circulator. A stir bar is inserted into a franz diffusion cell. Tissue is placed over the franz diffusion cell, and it is ensured that the tissue covers the entire area with an overlap onto a glass joint. The top of a diffusion cell is placed over the tissue, and the top of the cell is clamped to the bottom. About 5 mL of receptor media is loaded into the receiver area to ensure that no air bubbles are trapped in the received portion of the cell. This ensures that all 5 mL can fit into the receiver area. Stirring is begun, and temperature is allowed to equilibrate for about 20 minutes. Meanwhile, High Performance Liquid Chromatography (HPLC) vials are labelled by cell number and time point. One must then check again for air bubbles as the solution will degas during heating.

If testing films, one can perform the following next steps: (1) weigh films, punch to match diffusion area (or smaller), reweigh, record pre- and post-punching weight; (2) wet a donor area with approximately 100 μL of phosphate buffer; (3) place film on a donor surface, top with 400 μL of phosphate buffer, and start timers.

For solution studies, one can perform the following steps: (1) using a micropipette, dispense 500 μL of the solution into each donor cell, start the timers; (2) sample 200 μL at the following time points (time=0 min, 20 min, 40 min, 60 min, 120 min, 180 min, 240 min, 300 min, 360 min), and place in labelled HPLC vials, ensure no air is trapped in the bottom of the vial by tapping the closed vials; (3) replace each sample time with 200 μL of receptor media (to maintain 5 mL); (4) When all time points completed, disassemble the cells and dispose of all materials properly.

### Example 3

### Ex Vivo Permeation Evaluation

An exemplary ex vivo permeation evaluation is as follows.
1. Tissue is freshly excised and shipped (e.g. overnight) at 4° C.
2. The tissue is processed and frozen at −20° C. for up to three weeks prior to use.
3. The tissue is dermatomed to precise thickness.
4. Approximately 5 mL of receiving media is added to the receiving compartment. The media is selected to ensure sink conditions.
5. The tissue is placed in a franz diffusion cell, which includes a donor compound, a donor chamber, a membrane, sampling port, receptor chamber, stir bar, and a heater/circulator.
6. Approximately 0.5 mL of donor solution is applied or 8 mm circular film and wetted with 500 μL PBS buffer.
7. Samples are taken from the receiving chamber at given intervals and replaced with fresh media.

### Example 4

### Transbuccal Delivery of Doxepin

The following is an exemplary permeation study on the transbuccal delivery of doxepin. The studies were conducted under a protocol approved by the Animal Experimentation Ethics Committee of the University of Barcelona (Spain) and the Committee of Animal Experimentation of the regional autonomous government of Catalonia (Spain). Female pigs 3-4-months-old were used. The porcine buccal mucosa from the cheek region was excised immediately after the pigs were sacrificed in the animal facility at Bellvitge Campus (University of Barcelona, Spain) using an overdose of sodium thiopental anesthesia. The fresh buccal tissues were transferred from the hospital to the laboratory in containers filled with Hank's solution. The remaining tissue specimens were stored at −80° C. in containers with a PBS mixture containing 4% albumin and 10% DMSO as cryoprotective agents.

For the permeation studies, the porcine buccal mucosa was cut to 500+/−50 μm thick sheets, which contributes to the diffusional barrier (Buccal bioadhesive drug delivery A promising option for orally less efficient drugs Sudhakar et al., Journal of Controlled Release 114 (2006) 15-40), using an electric dermatome (GA 630, Aesculap, Tuttlingen, Germany) and trimmed with surgical scissors in adequate pieces. The majority of the underlying connective tissue was removed with a scalpel.

Membranes were then mounted in specially designed membrane holders with a permeation orifice diameter of 9 mm (diffusion area 0.636 cm²). Using the membrane holder, each porcine buccal membrane was mounted between the donor (1.5 mL) and the receptor (6 mL) compartments with the epithelium side facing the donor chamber and the connective tissue region facing the receiver of static Franz-type diffusion cells (Vidra Foc Barcelona, Spain) avoiding bubbles formation.

Infinite dose conditions were ensured by applying 100 μL as donor solution of a saturated doxepin solution into the receptor chamber and sealed by Parafilm immediately to prevent water evaporation. Prior to conducting the experiments, the diffusion cells were incubated for 1 h in a water bath to equalize the temperature in all cells (370+/−° C.). Each cell contained a small Teflon1 coated magnetic stir bar which was used to ensure that the fluid in the receptor compartment remained homogenous during the experiments.

Sink conditions were ensured in all experiments by initial testing of doxepin saturation concentration in the receptor medium. Samples (300 μL) were drawn via syringe from the center of the receptor compartment at pre-selected time intervals (0.1, 0.2, 0.3, 0.7, 1, 2, 3, 4, 5 and 6 h) for 6 h. The removed sample volume was immediately replaced with the same volume of fresh receptor medium (PBS; pH 7.4) with great care to avoid trapping air beneath the membrane.

Additional details can be found in A. Gimemo, et al. Transbuccal delivery of doxepin: Studies on permeation and histological evaluation, International Journal of Pharmaceutics 477 (2014), 650-654, which is incorporated by reference herein.

### Example 5

### Oral Transmucosal Delivery

Porcine oral mucosal tissue has similar histological characteristics to human oral mucosal tissue (Heaney T G, Jones R S, Histological investigation of the influence of adult porcine alveolar mucosal connective tissues on epithelial differentiation. Arch Oral Biol 23 (1978) 713-717; Squier C A, and Collins P, The relationship between soft tissue attachment, epithelial downgrowth and surface porosity. Journal of Periodontal Research 16 (1981) 434-440). Lesch et al. (The Permeability of Human Oral Mucosa and Skin to Water, J Dent Res 68 (9), 1345-1349, 1989) reported that the water permeability of porcine buccal mucosa was not significantly different from human buccal mucosa but the floor of the mouth was more permeable in human tissue than in pig tissue. Comparisons between fresh porcine tissue specimens and those stored at −80° C. revealed no significant effect on permeability as a result of freezing. Porcine buccal mucosal absorption has been studied for a wide range of drug molecules both in vitro and in vivo (see, e.g., Table 1 of M. Sattar, Oral transmucosal drug delivery—current status and future prospects, International Journal of Pharmaceutics 471 (2014) 498-506), which is incorporated by reference herein. Typically, in vitro studies involve mounting excised porcine buccal tissue in Ussing chambers, Franz cells or similar diffusion apparatus. The in vivo studies described in the literature involve the application of the drug as a solution, gel or composition to the buccal mucosa of pigs followed by plasma sampling.

Nicolazzo et al. (The Effect of Various in Vitro Conditions on the Permeability Characteristics of the Buccal Mucosa, Journal of Pharmaceutical Sciences 92(12) (2002) 2399-2410) investigated the effects of various in vitro conditions on the permeability of porcine buccal tissue using caffeine and oestradiol as model hydrophilic and lipophilic molecules. Drug permeation in the buccal mucosa was studied using modified Ussing chambers. Comparative permeation studies were performed through full thickness and epithelial tissues, fresh and frozen tissues. Tissue integrity was monitored by the absorption of the fluorescein isothiocyanate (FITC)-labeled dextran 20 kDa (FD20) and tissue viability was assessed using an MTT (3-[4,5-dimethylthiazol-2-yl]-2,5-diphenyltetrazolium bromide) biochemical assay and histological evaluation. Permeability through the buccal epithelium was 1.8-fold greater for caffeine and 16.7-fold greater for oestradiol compared with full thickness buccal tissue. Flux values for both compounds were comparable for fresh and frozen buccal epithelium although histological evaluation demonstrated signs of cellular death in frozen tissue. The tissue appeared to remain viable for up to 12 h postmortem using the MTT viability assay which was also confirmed by histological evaluation.

Kulkarni et al. investigated the relative contributions of the epithelium and connective tissue to the barrier properties of porcine buccal tissue. In vitro permeation studies were conducted with antipyrine, buspirone, bupivacaine and caffeine as model permeants. The permeability of the model diffusants across buccal mucosa with thickness of 250, 400, 500, 600, and 700 μm was determined. A bilayer membrane model was developed to delineate the relative contribution to the barrier function of the epithelium and the connective tissue. The relative contribution of the connective tissue region as a permeability barrier significantly increased with increasing mucosal tissue thickness. A mucosal tissue thickness of approximately 500 μm was recommended by the authors for in vitro transbuccal permeation studies as the epithelium represented the major permeability barrier for all diffusants at this thickness. The authors also investigated the effects of a number of biological and experimental variables on the permeability of the same group of model permeants in porcine buccal mucosa (Porcine buccal mucosa as in vitro model: effect of biological and experimental variables, Kulkarni et al., J Pharm Sci. 2010 99(3):1265-77). Significantly, higher permeability of the permeants was observed for the thinner region behind the lip (170-220 μm) compared with the thicker cheek (250-280 μm) region. Porcine buccal mucosa retained its integrity in Kreb's bicarbonate ringer solution at 4° C. for 24 h. Heat treatment to separate the epithelium from underlying connective tissue did not adversely affect its permeability and integrity characteristics compared with surgical separation.

Additional details can be found at M. Sattar, Oral transmucosal drug delivery—current status and future prospects, International Journal of Pharmaceutics 471 (2014) 498-506, which is incorporated by reference herein.

### Example 6

### Cryopreservation of Buccal Mucosa

Different areas of porcine buccal mucosa have different pattern of permeability, there is significantly higher permeability in the region behind the lips in comparison to cheek region, because in porcine buccal mucosa, the epithelium acts as a permeability barrier, and the thickness of the cheek epithelium is greater than that of the region behind the lips (Harris and Robinson, 1992). In exemplary permeation studies, the fresh or frozen porcine buccal mucosa from the same area was cut to 500±50 μm thick sheets, which contributes to the diffusional barrier (Sudhakar et al., 2006), were obtained using an electric dermatome (model GA 630, Aesculap, Tuttlingen, Germany) and trimmed with surgical scissors in adequate pieces. All devices utilized were previously sterilized. The majority of the underlying connective tissue was removed with a scalpel. Membranes were then mounted in specially designed membrane holders with a permeation orifice diameter of 9 mm (diffusion area 0.63 cm²). Using the membrane holder, each porcine buccal membrane was mounted between the donor (1.5 mL) and the receptor (6 mL) compartments with the epithelium facing the donor chamber and the connective tissue region facing the receiver of static Franz-type diffusion cells (Vidra Foc, Barcelona, Spain) avoiding bubbles formation. Experiments were performed using PP, which has lipophilic characteristics (log P=1.16; n-octanol/PBS, pH 7.4), ionisable (pKa=9.50) and a MW=259.3 g/mol, as a model drug (Modamio et al., 2000).

Infinite dose conditions were ensured by applying 300 μL as a donor solution of a saturated solution of PP (C0=588005±5852 μg/mL at 37°±1° C., n=6), in PBS (pH 7.4) into the receptor chamber and sealed by Parafilm immediately to prevent water evaporation.

Prior to conducting the experiments, the diffusion cells were incubated for 1 h in a water bath to equilibrate the temperature in all cells (37°±1 C). Each cell contained a small Teflon coated magnetic stir bar which was used to ensure that the fluid in the receptor compartment remained homogenous during the experiments. Sink conditions were ensured in all experiments after initial testing of PP saturation concentration in the receptor medium.

Samples (300 μL) were drawn via syringe from the center of the receptor compartment at the following time intervals: 0.25, 0.5, 1, 2, 3, 4, 5 and 6 h. The removed sample volume was immediately replaced with the same volume of fresh receptor medium (PBS; pH 7.4) with great care to avoid trapping air beneath the dermis. Cumulative amounts of the drug (μg) penetrating the unit surface area of the mucosa membrane (cm²) were corrected for sample removal and plotted versus time (h). The diffusion experiments were carried out 27 times for the fresh and 22 times for the frozen buccal mucosa.

Additional details can be found at S. Amores, An improved cryopreservation method for porcine buccal mucosa in ex vivo drug permeation studies using Franz diffusion cells, European Journal of Pharmaceutical Sciences 60 (2014) 49-54.

### Example 7

### Permeation of Quinine Across Sublingual Mucosa Sections

Since porcine and human oral membranes are similar in composition, structure and permeability measurements, porcine oral mucosa is a suitable model for human oral mucosa. Permeability across the porcine oral mucosa is not metabolically linked therefore it is not important for the tissue to be viable.

To prepare the porcine membranes, porcine floor of mouth and ventral (underside) tongue mucosa membranes were excised by blunt dissection using a scalpel. The excised mucosa were cut into approximately 1 cm squares and frozen on aluminum foil at −20° C. until used (<2 weeks). For non-frozen ventral surface of porcine tongue, the mucosa was used in the permeation studies within 3 h of excision.

The permeability of the membranes to quinine was determined using all-glass Franz diffusion cells with a nominal receptor volume of 3.6 mL and diffusional area of 0.2 cm². The cell flanges were greased with high performance vacuum grease and the membranes mounted between the receptor and donor compartments, with the mucosal surface uppermost. Clamps were used to hold the membranes into position before the receptor compartments were filled with degassed phosphate buffered saline (PBS), pH 7.4. Micromagnetic stirrer bars were added to the receptor compartments and the complete cells were placed in a water bath at 37° C. The membranes were equilibrated with PBS applied to the donor compartments for 20 min before being aspirated with a pipette. Aliquots of 5 μL of the quinine solution or 100 μL of the saturated solutions of Q/2-HP-β-CD complex in different vehicles were applied to each of the donor compartments. In the study to determine the effect of saliva on the permeation of quinine across the ventral surface of the tongue, 100 μL of sterile saliva was added to the donor compartments before adding 5 μL of the quinine solution.

At 2, 4, 6, 8, 10 and 12 h, the receptor phases were withdrawn from the sampling ports and aliquots of 1 mL samples were transferred to HPLC autosampler vials, before being replaced with fresh PBS stored at 37° C. Apart from the studies involving Q/2-HIP-β-CD saturated solutions (where an infinite dose was applied at the start of the experiments), 5 μL of the respective quinine solution was reapplied to the donor phase up to 10 h. The purpose of this was to represent a hypothetical in-use finite dosing regimen based upon an interval of 2 h between doses. At least 3 replicates were carried out for each study.

Additional details can be found at C. Ong, Permeation of quinine across sublingual mucosa, in vitro, International Journal of Pharmaceutics 366 (2009) 58-64.

### Example 8

### Ex-Vivo Initial Study—Form of the API

In this example, the permeation of Epinephrine Base was tested—solubilized in situ vs. the inherently soluble Epinephrine Bitartrate and no differences were found. Epinephrine Bitartrate was selected for further development based on ease of processing. Flux was derived as slope of the amount permeated as a function of time. Steady state flux extrapolated from the plateau of flux vs time curve multiplied by the volume of receiver media. The graph in FIG. 2A shows average amount permeated vs. time, with 8.00 mg/mL Epinephrine bitartrate and 4.4 mg/mL Epinephrine base solubilized. The graph in FIG. 2B shows average flux vs. time, with 8.00 mg/mL Epinephrine bitartrate and 4.4 mg/mL Epinephrine base solubilized.

| | Average Steady |
|---|---|
| | State Flux |
| Donor Solution | (ug/cm2*min) |
| Epinephrine Base (conc 4.4 mg/mL) | 0.512 |
| Epinephrine Bitartrate (conc 8.00 mg/mL) | 0.466 |

### Example 9

### Concentration Dependence on Permeation/Flux

In this study, ex-vivo permeation of Epinephrine Bitartrate as a function of concentration was studied. FIG. 3 shows ex-vivo permeation of epinephrine bitartrate as a function of concentration. The study compared concentrations of 4 mg/mL, 8 mg/mL, 16 mg/mL and 100 mg/mL. Results showed that increasing concentration resulted in increased permeation, and level of enhancement diminishes at higher loading. The study compared concentrations of 4 mg/mL, 8 mg/mL, 16 mg/mL and 100 mg/mL.

| | Average Steady |
|---|---|
| | State Flux |
| Donor Solution | (ug/cm2*min) |
| Epinephrine Bitartrate (conc 4 mg/mL) | 0.167 |
| Epinephrine Bitartrate (conc 8 mg/mL) | 0.466 |
| Epinephrine Bitartrate (conc 16 mg/mL) | 1.317 |
| Epinephrine Bitartrate (conc 100 mg/mL) | 2.942 |

| | Ratio of | Theoretical |
|---|---|---|
| Donor Solution | enhancement | enhancement |
| Epinephrine Bitartrate (4.00 mg/mL) | N/A | N/A |
| Epinephrine Bitartrate (8.00 mg/mL) | 2.8 | 2 |
| Epinephrine Bitartrate (16.00 mg/mL) | 7.9 | 4 |
| Epinephrine Bitartrate (100.00 mg/mL) | 17.6 | 25 |

### Example 10

### Influence of pH

In this example, the permeation of Epinephrine Bitartrate as a function of solution pH was studied. In this example, acidic conditions were explored for the ability to promote stability. The results showed that pH 5 was slightly more favorable as compared to pH 3. The inherent pH of epinephrine bitartrate in solution in the concentration ranges explored is 4.5-5. No pH adjustment with buffers was required.

FIG. 4 shows permeation of epinephrine bitartrate as a function of solution pH. Acidic conditions were explored to promote stability. The results compared Epinephrine Bitartrate pH 3 buffer and Epinephrine Bitartrate pH 5 buffer, and found that the Epinephrine Bitartrate pH 5 buffer was slightly favorable.

### Example 11

### Influence of Enhancers on Permeation of Epinephrine

In this example, the permeation of epinephrine to test for transmucosal delivery was studied as the amount permeated (pg) vs. time (in minutes). The following enhancers were screened for concentration effects in a solution containing 16.00 mg/mL Epinephrine. The graph on FIG. 5 demonstrates the results of these enhancers as a function of time.

| | | Average Steady | Percent |
|---|---|---|---|
| | | State Flux | Enhance- |
| Legend | Enhancer | (μg/cm²*min) | ment |
| No Enhancer | None | 1.317 | N/A |
| Enhancer A | 3% Labrasol | 5.208 | 395 |
| | Caprylocaproyl polyoxyl-8 | | |
| | glycerides | | |
| Enhancer B | 3% Propylene glycol | 2.385 | 181 |
| | monocaprylate | | |
| Enhancer C | 3% Polyglyceryl-3 oleate | 1.482 | 112 |
| Enhancer D | 3% Oleoyl polyoxyl-6 | 0.281 | 21 |
| | glycerides | | |
| Enhancer E | 3% TDM | 2.642 | 201 |
| Enhancer F | 3% SGDC | 0.342 | 26 |
| Enhancer G | 3% Lauroyl polyoxyl-32 | 1.641 | 125 |
| | glycerides | | |
| Enhancer H | 3% Ethanol | 0.163 | 12 |
| Enhancer I | 6% Ethanol | 0.254 | 19 |
| Enhancer J | 6% Labrasol | 4.444 | 337 |
| | Caprylocaproyl polyoxyl-8 | | |
| | glycerides | | |
| Enhancer K | 6% Polyglyceryl-3 oleate | 0.306 | 23 |
| Enhancer L | 3% clove oil | 8.216 | 624 |

Enhancers were selected and designed with functionality influencing different barriers in the mucosa. While all tested enhancers did improve the amount permeated over time, clove oil and Labrasol, in particular have shown significantly and unexpectedly high enhancement of permeation.

| | | set 2 (A, B, C) | | | | steady |
|---|---|---|---|---|---|---|
| | | average | std dev set | set 2 (D, E, F) | | state |
| | Time | amount | 2 amount | average flux | std dev | flux |
| | (min) | permeated | perm | (μg/cm²*min) | set 2 | average |
| 8.00 mg/0.500 mL | 30 | 0 | 0 | 0 | 0 | |
| 40 mg/strip | 45 | 2.5 | 4.33012702 | 0.260416667 | 0.4510549 | 1.317274 |
| no enhancer | 60 | 5.5 | 7.08872344 | 0.3125 | 0.3125 | |
| | 120 | 31.33333333 | 26.1549868 | 0.672743056 | 0.5050226 | |
| | 180 | 72.66666667 | 58.215834 | 1.076388889 | 0.8496863 | |
| | 240 | 112.1666667 | 80.1878004 | 1.028645833 | 0.5733604 | |
| | 300 | 160.1666667 | 103.254943 | 1.25 | 0.6036108 | |
| | 360 | 213.3333333 | 131.305306 | 1.384548611 | 0.7308311 | |

| | | set 1 (A, B, C) | | | | |
|---|---|---|---|---|---|---|
| | | average | | | | steady |
| | | amount | std dev set | set 1 (A, B, C) | | state |
| | Time | permeated | 1 amount | average flux | std dev | flux |
| | (min) | (ug) | perm | (μg/cm²*min) | set 1 | average |
| 8.00 mg/0.500 mL | 0 | 0 | 0 | 0 | 0 | |
| 40 mg/strip | 30 | 0.5 | 0.8660254 | 0.026041667 | 0.0451055 | 5.208333 |
| Labrasol | 45 | 2.666666667 | 0.28867513 | 0.225694444 | 0.0601407 | |
| | 60 | 4 | 1.80277564 | 0.138888889 | 0.1674245 | |
| | 120 | 28 | 16.3935963 | 0.625 | 0.3836177 | |
| | 180 | 98 | 53.3291665 | 1.822916667 | 0.9701176 | |
| | 240 | 238.1666667 | 93.0017921 | 3.650173611 | 1.136722 | |
| | 300 | 421.1666667 | 115.153521 | 4.765625 | 0.6675003 | |
| | 360 | 638.1666667 | 130.709921 | 5.651041667 | 0.4732495 | |

| | | set 2 (A, B, C) | | | | steady |
|---|---|---|---|---|---|---|
| | | average | std dev set | set 2 (D, E, F) | | state |
| | Time | amount | 2 amount | average flux | std dev | flux |
| | (min) | permeated | perm | (μg/mL*min) | set 2 | average |
| 8.00 mg/0.500 mL | 0 | 0 | 0 | 0 | 0 | |
| 40 mg/strip | 30 | 0.00 | 0 | 0 | 0 | |
| capryol 90 | 45 | 0.00 | 0 | 0 | 0 | 1.931424 |
| | 60 | 0.00 | 0 | 0 | 0 | |
| | 120 | 9.17 | 5.79511288 | 0.238715278 | 0.1509144 | |
| | 180 | 38.67 | 16.7655401 | 0.768229167 | 0.2864583 | |
| | 240 | 88.50 | 30.2654919 | 1.297743056 | 0.4018216 | |
| | 300 | 150.67 | 39.6936183 | 1.618923611 | 0.3269068 | |
| | 360 | 236.83 | 51.9358579 | 2.243923611 | 0.4999616 | |

| | | set 2 (A, B, C) | | | | steady |
|---|---|---|---|---|---|---|
| | | average | std dev set | set 2 (D, E, F) | | state |
| | Time | amount | 2 amount | average flux | std dev | flux |
| | (min) | permeated | perm | (μg/mL*min) | set 2 | average |
| 8.00 mg/0.500 mL | 0 | 0.8 | 1.78885438 | 0 | 0 | |
| 40 mg/strip | 30 | 10.80 | 13.325727 | 0.520833333 | 0.7186273 | |
| plurol oleique | 45 | 20.90 | 22.1624683 | 1.052083333 | 0.9356173 | 1.481771 |
| | 60 | 33.00 | 30.8058436 | 1.260416667 | 0.9319861 | |
| | 120 | 90.70 | 68.1951245 | 1.502604167 | 1.005753 | |
| | 180 | 157.00 | 107.373763 | 1.7265625 | 1.0427891 | |
| | 240 | 239.80 | 140.586539 | 2.15625 | 1.2085059 | |
| | 300 | 285.60 | 184.236397 | 1.192708333 | 1.484335 | |
| | 360 | 353.60 | 221.81676 | 1.770833333 | 0.993644 | |

| | | set 2 (A, B, C) | | | | steady |
|---|---|---|---|---|---|---|
| | | average | std dev set | set 2 (D, E, F) | | state |
| | Time | amount | 2 amount | average flux | std dev | flux |
| | (min) | permeated | perm | (μg/mL*min) | set 2 | average |
| 8.00 mg/0.500 mL | 0 | 0.00 | 0.00 | 0.00 | 0.00 | |
| 40 mg/strip | 20 | 0.00 | 0.00 | 0.00 | 0.00 | |
| TDM | 40 | 3.00 | 2.43 | 0.23 | 0.32 | 2.642144 |
| | 60 | 8.83 | 4.86 | 0.46 | 0.35 | |
| | 120 | 41.33 | 15.08 | 0.85 | 0.49 | |
| | 180 | 99.75 | 30.17 | 1.52 | 0.79 | |
| | 240 | 179.92 | 48.30 | 2.09 | 0.98 | |
| | 300 | 276.92 | 72.35 | 2.53 | 1.19 | |
| | 360 | 382.83 | 102.02 | 2.76 | 1.38 | |

| | | set 2 (A, B, C) | | | | steady |
|---|---|---|---|---|---|---|
| | | average | std dev set | set 2 (D, E, F) | | state |
| | Time | amount | 2 amount | average flux | std dev | flux |
| | (min) | permeated | perm | (μg/mL*min) | set 2 | average |
| 8.00 mg/0.500 mL | 0 | 0 | 0 | 0 | 0 | |
| 40 mg/strip | 30 | 0.00 | 0 | 0 | 0 | |
| SGDC | 45 | 0.67 | 1.30384048 | 0.069444444 | 0.1261521 | 0.341797 |
| | 60 | 1.58 | 1.94935887 | 0.095486111 | 0.1063147 | |
| | 120 | 7.00 | 9.44325156 | 0.141059028 | 0.1910856 | |
| | 180 | 16.17 | 22.0323626 | 0.238715278 | 0.316132 | |
| | 240 | 28.58 | 37.6191441 | 0.323350694 | 0.3977149 | |
| | 300 | 43.00 | 54.3927844 | 0.375434028 | 0.4112124 | |
| | 360 | 54.83 | 65.7976063 | 0.308159722 | 0.3008536 | |

| | | set 2 (A, B, C) | | | | steady |
|---|---|---|---|---|---|---|
| | | average | std dev set | set 2 (D, E, F) | | state |
| | Time | amount | 2 amount | average flux | std dev | flux |
| | (min) | permeated | perm | (μg/mL*min) | set 2 | average |
| 8.00 mg/0.500 mL | 0 | 0.8 | 1.15108644 | 0 | 0 | |
| 40 mg/strip | 30 | 1.10 | 1.51657509 | 0.015625 | 0.0232924 | |
| Labrafil | 45 | 1.10 | 1.51657509 | 0 | 0 | 0.28125 |
| | 60 | 1.10 | 1.51657509 | 0 | 0 | |
| | 120 | 4.00 | 4.89897949 | 0.075520833 | 0.0984775 | |
| | 180 | 9.10 | 10.9167303 | 0.1328125 | 0.1671035 | |
| | 240 | 15.20 | 18.7169709 | 0.158854167 | 0.2034753 | |
| | 300 | 25.70 | 29.8487856 | 0.2734375 | 0.2910091 | |
| | 360 | 36.80 | 43.3093523 | 0.2890625 | 0.3532943 | |

| | | set 2 (A, B, C) | | | | steady |
|---|---|---|---|---|---|---|
| | | average | std dev set | set 2 (D, E, F) | | state |
| | Time | amount | 2 amount | average flux | std dev | flux |
| | (min) | permeated | perm | (μg/mL*min) | set 2 | average |
| 8.00 mg/0.500 mL | 0 | 0 | 0 | 0 | 0 | |
| 40 mg/strip | 20 | 0.33 | 0.89442719 | 0.026041667 | 0.0637888 | |
| Gelucire 44/14 | 40 | 3.83 | 5.94138031 | 0.2734375 | 0.378725 | 1.629774 |
| | 60 | 11.50 | 17.1850225 | 0.598958333 | 0.8405853 | |
| | 120 | 41.58 | 48.5059275 | 0.783420139 | 0.8042794 | |
| | 180 | 91.92 | 82.5124233 | 1.310763889 | 0.9525224 | |
| | 240 | 150.17 | 118.949569 | 1.516927083 | 0.9914576 | |
| | 300 | 217.50 | 158.792947 | 1.753472222 | 1.0756081 | |
| | 360 | 275.33 | 189.967563 | 1.506076389 | 0.9083155 | |

| | | set 2 (A, B, C) | | | | steady |
|---|---|---|---|---|---|---|
| | | average | std dev set | set 2 (D, E, F) | | state |
| | Time | amount | 2 amount | average flux | std dev | flux |
| | (min) | permeated | perm | (μg/cm²*min) | set 2 | average |
| 8.00 mg/0.500 mL | 0 | 0 | 0 | 0 | 0 | |
| 40 mg/strip | 20 | 0 | 0 | 0 | 0 | |
| clove oil | 40 | 28.66666667 | 27.360251 | 2.239583333 | 2.1375196 | 8.270399 |
| | 60 | 96.5 | 79.5424415 | 5.299479167 | 4.0954816 | |
| | 120 | 389.6666667 | 278.072533 | 7.634548611 | 5.2070528 | |
| | 180 | 688.6666667 | 451.678628 | 7.786458333 | 4.5210426 | |
| | 240 | 1009.166667 | 603.252089 | 8.346354167 | 3.9590828 | |
| | 300 | 1333.5 | 759.653046 | 8.446180556 | 4.1168559 | |
| | 360 | 1644.333333 | 878.2762 | 8.094618056 | 3.2301203 | |

| | | set 2 (A, B, C) | | | | steady |
|---|---|---|---|---|---|---|
| | | average | std dev set | set 2 (D, E, F) | | state |
| | Time | amount | 2 amount | average flux | std dev | flux |
| | (min) | permeated | perm | (μg/mL*min) | set 2 | average |
| 8.00 mg/0.500 mL | 0 | 0 | 0 | 0 | 0 | |
| 40 mg/strip | 20 | 0 | 0 | 0 | 0 | 3.161892 |
| 3% Labrasol + | 40 | 1.666666667 | 2.88675135 | 0.130208333 | 0.2255274 | |
| 1% TDM | 60 | 6.833333333 | 11.8356805 | 0.403645833 | 0.6991351 | |
| | 120 | 68.83333333 | 92.6907942 | 1.614583333 | 2.1084505 | |
| | 180 | 103.6666667 | 101.539812 | 0.907118056 | 0.3351021 | |
| | 240 | 180 | 130.184484 | 1.987847222 | 0.7476876 | |
| | 300 | 291.5 | 149.81572 | 2.903645833 | 0.5193664 | |
| | 360 | 422.8333333 | 164.032263 | 3.420138889 | 0.5530917 | |

### Example 12

### Impact of Enhancers on Epinephrine Release

Release profiles of epiphrine were studied to determine the impact of enhancers (Labrasol and clove oil) on epinephrine release. FIG. 6A shows the epinephrine release from different polymer platforms. FIG. 6B shows the impact of enhancers on epinephrine release. The results showed that the amount permeated leveled off after about 40 minutes to be between approximately 3250 and 4250 pg. The tested enhancers were shown not to restrict the release of epinephrine from the matrix.

### Example 13

### Accelerated Stability

The stabilizer loading variants were tested.

| | | Formulation | Formulation |
|---|---|---|---|
| Time (weeks) | Formulation | 10 with 0.25% | 10 with 1% |
| @ 40° C./ | 10 | Stabilizer | Stabilizer |
| 75% R.H. | EPI mg/film | EPI mg/film | EPI mg/film |
| 0 | 39.3 | 37.9 | 38.3 |
| 2 | 35.2 | 36.8 | 34.7 |
| 4 | 38.2 | 36.8 | 36.2 |
| 8 | 37.7 | 35.6 | 35.1 |
| 12 | 36.1 | 35.4 | 35.1 |

### Example 14

### Impact of Enhancer

A pharmacokinetic model in the male Yucatan, miniature swine was studied. The graph on FIG. 7 shows the results of a pharmacokinetic model in the male Yucatan, miniature swine. The study compares a 0.3 mg Epipen, a 0.12 mg Epinephrine IV and a placebo.

The impact of no enhancer is shown in FIG. 8 on the concentration profiles of a 0.3 mg Epipen and a 40 mg Epinephrine film with no enhancer

The impact of enhancer 3% Labrasol is shown in FIG. 9, which shows the impact of Enhancer A (Labrasol) on the concentration profiles of a 40 mg Epinephrine film vs. a 0.3 mg Epipen. FIG. 10 shows the impact of Enhancer L (clove oil) on the concentration profiles of two 40 mg Epinephrine films (10-1-1) and (11-1-1) vs. a 0.3 mg Epipen.

In addition, the influence of film dimensions and impact of clove oil (3%) is also shown in FIG. 11. This study was carried out comparing 0.30 mg EpiPen (n=4), a 40 mg Epinephrine Film (10-1-1) (n=5) and a 40 mg Epinephrine Film (11-1-1) (n=5). The concentration vs. time profiles followed subligual or intramuscular epinephrine administration to male miniature swine.

Studies were performed to vary the ratio of ephinephrine to an enhancer. These studies were also concentration vs. time profiles following subligual or intramuscular epinephrine administration to male miniature swine. Varying the ratio of Epinephrine to clove oil (Enhancer L) produced the results shown in FIG. 12. This study was carried out comparing 0.30 mg EpiPen (n=4), a 40 mg Epinephrine Film (12-1-1) (n=5) and a 20 mg Epinephrine Film (13-1-1) (n=5).

### Example 15

A varying dose was carried out in constant matrix with enhancer Labrasol (3%) and clove oil (3%) are shown in FIGS. 13 and 14 respectively. The study in FIG. 13 was carried out comparing 0.30 mg EpiPen (n=4), a 40 mg Epinephrine Film (18-1-1) (n=5) and a 30 mg Epinephrine Film (20-1-1) (n=5). The study in FIG. 14 was carried out comparing 0.30 mg EpiPen (n=4), a 40 mg Epinephrine Film (19-1-1) (n=5) and a 30 mg Epinephrine Film (21-1-1) (n=5). These studies were also concentration vs. time profiles following subligual or intramuscular epinephrine administration to male miniature swine.

### Example 16

A pharmacokinetic model in the male miniature swine was studied to determine the impact of an enhancer (farnesol) on epinephrine concentration over time. The graph on FIG. 15 shows the epinephrine plasma concentration (in ng/mL) as a function of time (in minutes) following sublingual or intramuscular administration of a farnesol permeation enhancer. This study compares a 0.3 mg Epipen (n=3), a 30 mg Epinephrine Film 31-1-1 (n=5) and a 30 mg Epinephrine Film 32-1-1 (n=5) each Epinephrine Film being formulated with a farnesol enhancer. As shown in this figure, the 31-1-1 film demonstrates enhanced stability of epinephrine concentration starting at about 30-40 minutes until approximately 130 minutes.

The graph in FIG. 16 is taken from the same study as FIG. 17, but shows exclusively the data points comparing the 0.3 mg Epipen against the 30 mg Epinephrine Film 31-1-1 (n=5).

The graph in FIG. 17 is taken from the same study as FIG. 17, but shows exclusively the data points comparing the 0.3 mg Epipen against the 30 mg Epinephrine Film 32-1-1 (n=5).

### Example 17

Referring to FIG. 18, this graph shows a pharmacokinetic model in the male miniature swine was studied to determine the impact of an enhancer (farnesol) on epinephrine concentration over time following sublingual or intramuscular administration. The epinephrine plasma concentration (in ng/mL) is shown as a function of time (in minutes) following sublingual or intramuscular administration of a farnesol permeation enhancer in Epinephrine films. The study compared data from three 0.3 mg Epipens against five 30 mg Epinephrine films (32-1-1). The data shows the Epinephrine films film having enhanced stability of epinephrine concentration starting at about 20-30 minutes until approximately 130 minutes.

### Example 18

In one embodiment, an epinephrine pharmaceutical composition film can be made with the following formulation:

| | Formulation A | | |
|---|---|---|---|
| MATERIAL | WT % dry | WT % wet | mg/Strip |
| EPINEPHRINE bitartrate | 46.40 | 18.56 | 54.56 |
| hydroxypropylmethyl cellulose | 11.54 | 4.61 | 13.57 |
| polyvinyl pyrrolidone | 27.92 | 11.17 | 32.84 |
| Glycerol monooleate | 0.58 | 0.23 | 0.68 |
| Polyethylene Oxide | 1.16 | 0.46 | 1.36 |
| Polysorbate | 0.58 | 0.23 | 0.68 |
| Phytoextract | 9.98 | 3.99 | 9.97 |
| Stabilizer | 0.12 | 0.05 | 0.14 |
| Buffer | 0.58 | 0.23 | 0.68 |
| Artificial sweetener | 1.16 | 0.46 | 1.36 |
| Linoleic acid | 0.0037 | 0.00 | 0.00 |
| Farnesol | | | |
| Yellow # 5 | | | |
| TOTAL | 100.00 | 40.00 | 115.84 |

### Example 19

An epinephrine pharmaceutical film compositions was made with the following formulation:

| | Formulation B | | |
|---|---|---|---|
| MATERIAL | WT % dry | WT % wet | mg/Strip |
| EPINEPHRINE bitartrate | 46.17 | 18.47 | 54.29 |
| Hydroxypropylmethyl cellulose | 11.48 | 4.59 | 13.50 |
| Polyvinyl pyrrolidone | 27.78 | 11.11 | 32.67 |
| Glycerol monooleate | 0.58 | 0.23 | 0.68 |
| Polyethylene Oxide | 1.15 | 0.46 | 1.35 |
| Polysorbate | 0.58 | 0.23 | 0.68 |
| Phytoextract | 9.93 | 3.97 | 9.92 |
| Stabilizer | 0.12 | 0.05 | 0.14 |
| Buffer | 0.58 | 0.23 | 0.68 |
| Artificial sweetener | 1.15 | 0.46 | 1.35 |
| Linoleic acid | 0.50 | 0.20 | 0.59 |
| Farnesol | | | |
| Yellow # 5 | | | |
| TOTAL | 100.00 | 40.00 | 115.85 |

### Example 20

In another embodiment, pharmaceutical film compositions were made with the following formulation:

| | Formulation C | | |
|---|---|---|---|
| MATERIAL | WT % dry | WT % wet | mg/Strip |
| EPINEPHRINE bitartrate | 46.35 | 18.54 | 54.51 |
| Hydroxypropylmethyl cellulose | 11.53 | 4.61 | 13.55 |
| Polyvinyl pyrrolidone | 27.90 | 11.16 | 32.80 |
| Glycerol monooleate | 0.58 | 0.23 | 0.68 |
| Polyethylene oxide | 1.16 | 0.46 | 1.36 |
| Polysorbate | 0.58 | 0.23 | 0.68 |
| Phytoextract | 9.97 | 3.99 | 9.96 |
| Stabilizer | 0.12 | 0.05 | 0.14 |
| Buffer | 0.58 | 0.23 | 0.68 |
| Artificial sweetener | 1.16 | 0.46 | 1.36 |
| Linoleic acid | | | |
| Farnesol | 0.10 | 0.04 | 0.06 |
| Yellow # 5 | | | |
| TOTAL | 100.00 | 40.00 | 115.78 |

### Example 21

In another embodiment, pharmaceutical film compositions were made with the following formulation:

| | Formulation D | | |
|---|---|---|---|
| MATERIAL | WT % dry | WT % wet | mg/Strip |
| EPINEPHRINE bitartrate | 46.07 | 18.43 | 54.52 |
| Hydroxypropylmethyl cellulose | 11.46 | 4.58 | 13.56 |
| Polyvinyl pyrrolidone | 27.73 | 11.09 | 32.81 |
| Glycerol monooleate | 0.57 | 0.23 | 0.68 |
| Polyethylene oxide | 1.15 | 0.46 | 1.36 |
| Polysorbate | 0.57 | 0.23 | 0.68 |
| Phytoextract | 9.91 | 3.96 | 9.96 |
| Stabilizer | 0.11 | 0.05 | 0.14 |
| Buffer | 0.57 | 0.23 | 0.68 |
| Artificial sweetener | 1.15 | 0.46 | 1.36 |
| Linoleic acid | 0.10 | 0.04 | 0.06 |
| Farnesol | 0.50 | 0.20 | 0.29 |
| Yellow # 5 | 0.10 | 0.04 | 0.06 |
| TOTAL | 100.00 | 40.00 | 116.16 |

### Example 22

Referring to FIG. 19, this graph shows a pharmacokinetic model (logarithmic scale) in the male miniature swine studied to determine the impact of an enhancer (600 clove oil and 6% Labrasol) on epinephrine plasma concentration over time following sublingual or intramuscular administration. The epinephrine plasma concentration (in ng/mL) is shown as a function of time (in minutes) following sublingual or intramuscular administration of a farnesol permeation enhancer in Epinephrine films. The data shows the Epinephrine films film having enhanced stability of epinephrine concentration starting at just after the 10 minute time point through about 30 minutes, and until approximately 100 minutes.

Referring to FIG. 20, this graph shows a pharmacokinetic model of the Epinephrine film formulation in the male miniature swine as referenced in FIG. 19 compared against the average data collected from a 0.3 mg Epipen (indicated in diamond data points). As the data indicates, the average plasma concentration for the 0.3 mg Epipen peaked between 0.5 and 1 ng/mL. By contrast, the Epinephrine film formulation peaked between 4 and 4.5 ng/mL.

### Example 23A

Referring to FIG. 21, this graph shows a pharmacokinetic model in the male miniature swine studied to determine the impact of an enhancer (9% clove+3% Labrasol) on epinephrine concentration over time following sublingual or intramuscular administration across 7 animal models. The general peak concentration was achieved between 10-30 minutes.

### Example 23B

In one embodiment, a dipivefrin pharmaceutical composition film can be made with the following formulation:

| MATERIAL | WT % dry | mg/Strip |
|---|---|---|
| Dipivefrin | 3.45 | 3.297 |
| HPMC E15 | 23.22 | 22.165 |
| PVP K90 | 57.57 | 54.959 |
| GMO | 0.50 | 0.476 |
| PEO N10 | 0.50 | 0.476 |
| PEG400 | 9.25 | 8.830 |
| clove oil | 3.00 | 2.437 |
| sodium citrate | 0.50 | 0.476 |
| sucralose | 1.00 | 0.952 |
| labrasol | 1.00 | 0.956 |
| green #3 | 0.01 | 0.010 |
| TOTAL | 100.00 | 95.03 |

### Example 24

In another embodiment, a dipivefrin pharmaceutical composition film can be made with the following formulation:

| MATERIAL | WT % dry | mg/Strip |
|---|---|---|
| Dipivefrin | 6.00 | 6.602 |
| HPMC | 21.22 | 23.350 |
| PVP | 51.52 | 56.690 |
| GMO | 0.50 | 0.548 |
| PEO | 0.50 | 0.548 |
| PEG400 | 9.25 | 10.179 |
| clove oil | 3.00 | 2.809 |
| citric acid | 3.00 | 3.301 |
| sodium citrate | 2.00 | 2.201 |
| sodium metabisulfite | 1.00 | 1.100 |
| sucralose | 1.00 | 1.097 |
| labrasol | 1.00 | 1.102 |
| green #3 | 0.01 | 0.011 |
| TOTAL | 100.00 | 109.54 |

### Example 25

In another embodiment, a dipivefrin pharmaceutical composition film can be made with the following formulation:

| MATERIAL | WT % dry | mg/Strip |
|---|---|---|
| Dipivefrin | 10.50 | 13.204 |
| HPMC | 20.22 | 25.427 |
| PVP | 48.02 | 60.385 |
| GMO | 0.50 | 0.627 |
| PEO | 0.50 | 0.627 |
| PEG400 | 9.25 | 11.633 |
| clove oil | 3.00 | 3.211 |
| citric acid | 3.00 | 3.773 |
| sodium citrate | 2.00 | 2.515 |
| sodium metabisulfite | 1.00 | 1.258 |
| sucralose | 1.00 | 1.254 |
| labrasol | 1.00 | 1.259 |
| green #3 | 0.01 | 0.013 |
| TOTAL | 100.00 | 125.18 |

### Example 26

In one study, the investigators compared the pharmacokinetics of epinephrine after Dipivefrin SL administration to other routes of administrations (Oral, SC and IV), specifically analyzing epinephrine bioavailability and conversion of dipivefrin to epinephrine.

| | | | | | | | Target Dose | |
|---|---|---|---|---|---|---|---|---|
| | No. of | | | Target Dose | Dipi Dose | | Amount (mL | |
| | Animals | | Dose | Level | Conc. (mg/DF | | or DF/ | |
| Group | (Male) | Test Article | Route | (mg/animal) | or mL) | | animal) | |
| 1 | 1 | Placebo | SL | 0 | 0 | mg/DF | 1 | DF |
| 2 | 5 | Dipivefrin DF: | SL | 6 | 6 | mg/DF | 1 | DF |
| | | Dip 16-1-1 | | | | | | |
| 3 | 3 | Dipivefrin | PO | 6 | 0.6 | mg/mL | 10 | mL |
| | | Solution | | | | | | |
| 4 | 3 | Dipivefrin | PO | 6 | 0.6 | mg/mL | 10 | mL |
| | | solution | | | | | | |
| | | (Dissolved SL | | | | | | |
| | | Film) | | | | | | |
| 5 | 4 | Dipivefrin | SC | 22 | 22 | mg/mL | 1 | mL |
| | | Solution | | | | | | |
| 6 | 4 | Dipivefrin | IV | 1 | 1 | mg/mL | 1 | mL |

Solution

The study compared various dose routes: sublingual (SL), per oral tablet (PO), subcutaneous (SC) and intravenous (IV). The PK Time-points (for All Groups): 0 (pre-dose), 2, 5, 10, 12, 15, 17, 20, 25, 30, 40, 60, 90 and 120 minutes and 3 hr, 4 hr, 6 hr and 8 hr post dose. An Oral Irritation assessment was performed (SL film group): Predose (0) and 24 hr postdose by Draize scoring. The dipivefrin film (DF) is an exemplary embodiment of the claimed pharmaceutical composition.

Referring to FIG. 22A, the data shows average epinephrine plasma concentration vs. time in 60 minutes. Referring to FIG. 22B, the data shows average epinephrine plasma concentration vs. time in 480 minutes. Surprisingly, the dipivefrin film showed ability to meet target Cmax of 0.2 ng/ml-1.5 ng/ml in less than 30 minutes. The Cmax was shown to be 0.1 ng/ml-2 ng/ml, 0.15 ng/ml-1.5 ng/ml, and 0.2 ng/ml-1.0 ng/ml. The Cmax was shown to be greater than 0.1 ng/ml, greater than 0.15 ng/ml, greater than 0.2 ng/ml, greater than 0.4 ng/ml, greater than 0.5 ng/ml, greater than 0.6 ng/ml, greater than 0.7 ng/ml and less than 2 ng/ml and less than 1.5 ng/ml.

The Tmax is shown in the range of 0-480 minutes, including 10-60 minutes, 20-40 minutes, 12-15 minutes, and 5-10 minutes. The Tmax has been shown to be less than 25 minutes, less than 20 minutes, 15 minutes, less than 12 minutes, and less than 10 minutes.

Referring to FIG. 23A, the data shows average dipivefrin plasma concentration vs. time in 60 minutes. Referring to FIG. 23A, the data shows average dipivefrin plasma concentration vs. time in 480 minutes. The amount of dipivefrin is shown to decrease as epinephrine is being formed. This data shows a surprising ability of the subject film to meet a target Cmax of 0.2 ng/ml-1.5 ng/ml and a Tmax of less than 35 minutes. The Cmax is shown to be 0.1 ng/ml-2 ng/ml, 0.15 ng/ml-25 ng/ml, 0.2 ng/ml-1.0 ng/ml, 0.2 ng/ml-1.2 ng/ml, and 0.2 ng/ml-1.3 ng/ml. The Cmax has been shown to be greater than 0.1 ng/ml, greater than 0.15 ng/ml, greater than 0.2 ng/ml, greater than 0.4 ng/ml, greater than 0.5 ng/ml, greater than 1.0 ng/ml, greater than 1.2 ng/ml, and less than 3 ng/ml, less than 2 ng/ml and less than 1.5 ng/ml. The Tmax is shown in the range of 0-480 minutes, including 10-60 minutes, 20-40 minutes, 12-15 minutes, and 5-10 minutes. The Tmax has been shown to be less than 25 minutes, less than 20 minutes, 15 minutes, less than 12 minutes, and less than 10 minutes.

Referring to FIG. 24, the data shows that average plasma concentration of epinephrine (dipivefrin SL 16-1-1 E) in round data points and dipivefrin (dipivefrin SL-Dip 16-1-1 D) in square data points. This shows the amount of dipivefrin decreases as epinephrine is being formed.

Referring to FIG. 25, the graph shows the conversion of dipivefrin to epinephrine (SC and IV groups). The dashed lines indicate dipivefrin, and the solid lines indicate epinephrine.

| | | | EPI | | | DIPI |
|---|---|---|---|---|---|---|
| | | EPI | Cmax | | DIPI | Cmax |
| | EPI | Tmax | (mean, | DIPI | Tmax | (mean, |
| | AUC0-t | (Min) | ng/ml) | AUC0-t | (Min) | ng/ml) |
| Placebo SL | 0 | 0 | 0 | 0 | 0 | 0 |
| Dipi 9% C6 mg | 197.4 | 180 | 0.75 | 12.26 | 30 | 1.14 |
| Dipi Solution | 41.4 | 120 | 0.28 | 0 | 0 | 0 |
| (6 mg, PO) | | | | | | |
| Dipi Film | 24.2 | 12 | 0.24 | 0 | 0 | 0 |
| Solution (6 mg, | | | | | | |
| PO) | | | | | | |
| Dipi Solution | 4220.0 | 180 (3^{rd} | 12.5 | 1505 | 5 | 15 |
| (22 mg, SC) | | peak) | | | | |
| Dipi Solution | 146.4 | 2 | 7.62 | 53.24 | 2 | 11.7 |
| (1 mg, IV) | | | | | | |
| % Boiavailability- | 131.0% | | | 128.5% | | |
| Dipi solution SC | | | | | | |
| (Relative to IV) | | | | | | |

The results showed no local irritation with the Dipivefrin SL film. Half-life or hydrolysis by enzyme is reflected in the conversion rate of dipivefrin to epinephrine. In certain embodiments, the half-life is less than 1 minute. In other embodiments, the half-life is so rapid that it may be seconds or fractions of a second. In some cases, plasma assays for the prodrug may not be feasible but are reflected in the emergence of neatepinephrine plasma levels not attributed to endogenous epinephrine. The results also showed that dipivefrin was converted into Epinephrine rapidly-SC administration of dipivefrin had a depot effect-had the higher and prolonged epinephrine exposure compared to IV administration. The results showed that dipivefrin IV administration had a faster onset and maximum epinephrine exposure at 2 minutes and cleared from the system within 2 hours. The results indicate that oral administration showed minimal epinephrine exposure compared to other routes of administration (dipivefrin concentration was below the detection limit). In short, surprisingly, no difference was found between dipivefrin solution and dipivefrin film solution with permeation enhancers.

### Example 27

In this investigation, the investigators studied the conversion of dipivefrin to epinephrine. The study adjusted three variables systemically: drug concentration, dose, and enhancer loading. The data was analyzed to identify speed to onset, Cmax, area under the plasma drug concentration-time curve (AUC) during the first 20-25 minutes.

Referring to FIG. 26, the data shows average epinephrine profiles across all matrix studies. The time to initial peak average is within about 30 minutes, 25 minutes, and 20 minutes. The 6 mg dipivefrin/6% clove film (dip 10-1-1) achieved the fastest average results.

| | | minutes | | | | | | | | | |
|---|---|---|---|---|---|---|---|---|---|---|---|
| | dose/clove | −10 | 5 | 10 | 12 | 15 | 17 | 20 | 25 | 30 | average |
| Dip 4-1-2 | 3 mg/3% | | | | 1 | 2 | 1 | | | 1 | 17.8 |
| Dip 4-2-1 | 6 mg/3% | | | 2 | 1 | 1 | | | | | 11.75 |
| Dip 10-1-1 | 6 mg/6% | | 1 | 4 | | | | | | | 9 |
| Dip 11-1-1 | 6 mg/6% | | | | | 1 | | 2 | 2 | | 21 |
| Dip 11-2-1 | 9 mg/6% | | | | | 2 | | | 2 | 1 | 22 |
| Dip 12-1-1 | 9 mg/9% | | | | | | 1 | 2 | | 1 | 21.75 |
| Dip 12-2-1 | 12 mg/9% | | | | 1 | 2 | | 2 | | | 16.4 |
| Dip 13-1-1 | 12 mg/6% | | | | | | 1 | 2 | 2 | | 21.4 |
| Dip 14-1-1 | 12 mg/6% | | | | | | 2 | 1 | 2 | | 20.8 |

Referring to FIG. 27, the data shows additional data comparing plasma concentration of dipivefrin and epinephrine (ng/ml) vs. time in hours. A mean therapeutic window has shown to be achieved within 30 minutes or less, including individual averages in 20 minutes or less, and 15 minutes or less.

### Example 28

An investigation yielded results that validate the receptor interactor antagonism needed to drive greater levels of epinephrine transmucosally. Preclinical results comparing the 30 mg formulation of epinephrine sublingual firm (ESF) versus a pretreatment of a phentolamine (a reversible nonselective α-adrenergic antagonist) at the site of application 10 min prior to dosing (phen 0.10 mg/ESF). The pretreatment showed that improved plasma concentration levels within a therapeutic window of 30 minutes or less, 25 minutes or less, and 20 minutes or less.

| | Tmax (min) | Cmax (ng/mL) | AUC (min*ng/mL) |
|---|---|---|---|
| Epipen | 35 | 1.52 + 0.857 | 74.48 + 22.94 |
| ESF | 25 | 0.99 + 0.64 | 42.96 + 11.04 |
| Phen 0.10 mg/ESF | 25 | 7.68 + 3.89 | 161.22 + 70.24 |

Referring to FIG. 28, this graph shows the epinephrine plasma concentration vs. time profile in a follow-up study that examined the loading of phentolamine and how it influences the absorption of epinephrine. For this study, 30 mg epinephrine sublingual films (ESF) were used. The results show that the loading is dose dependent. The results are shown in the graph and summarized below:

| | Tmax (min) | Cmax (ng/mL) | AUC (min*ng/mL) |
|---|---|---|---|
| Epipen | 35 | 2.04 + 2.13 | 90.35 + 105.0 |
| Phen 0.25 mg/ESF | 25 | 1.93 + 1.02 | 43.18 + 57.5 |
| Phen 0.10 mg/ESF | 27.5 | 1.19 + 0.60 | 45.12 + 27.24 |

As shown in the graph and indicated in the above data, a therapeutic window can be seen for ESF within 30 minutes or less, 25 minutes or less, and 20 minutes or less.

### Example 29

Referring to FIG. 29, this graph shows a mean baseline adjusted epinephrine concentration over 0-2 hours with varying residence times for a 12 mg pharmaceutical film. In this example, three different residence times were tested. The 4 minute residence time is shown in circle data points. The 2 minute residence time is shown in downward pointing triangle data points. The 0 minute residence time is shown in upward pointing triangle data points. The results are shown in the graph and summarized below.

| | AQST-109 | AQST-109 | AQST-109 | | |
|---|---|---|---|---|---|
| | 12 mg | 12 mg | 12 mg | | Epinephrine |
| | 4-minute | 2-minute | 0-minute | AQST-109 | IM Injection |
| | residence | residence | residence | 12 mg | 0.3 mg |
| | (N = 22 | (N = 23 | (N = 21 | (N = 48 | (N = 48 |
| Study Results | doses) | doses) | doses) | doses) | doses) |
| Arithmetic Mean | 678.4 | 663.9 | 359.8 | 426.1 | 396.7 |
| Cmax (pg/mL) | | | | | |
| Geometric Cmax | 350.4 | 303.9 | 211.2 | 274.3 | 350.6 |
| (pg/mL) | | | | | |
| AUC 0-10 minutes | 12.8 | 9.5 | 9.4 | 7.9 | 9.4 |
| (hr*pg/mL) | | | | | |
| AUC 0-20 minutes | 51.2 | 45.7 | 30.9 | 33.1 | 23.0 |
| (hr*pg/mL) | | | | | |
| AUC 0-30 minutes | 79.1 | 75.1 | 49.8 | 56.7 | 47.5 |
| (hr*pg/mL) | | | | | |

As shown in the graph and in the data above, AQST-109 12 mg continued to show rapid absorption with favorable pharmacokinetics across a variety of key metrics. The median time to maximum concentration (Tmax) was observed to be 12 minutes for AQST-109 at the 4-minute residence time, compared to 50 minutes for the epinephrine 0.3 mg intra-muscular (IM) injection from Part 2 of the study.

The Area Under the Curve (AUC) within the clinically relevant periods of 10 minutes, 20 minutes, and 30 minutes in each of three arms were comparable for both AQST-109 and the 0.3 mg IM injection. The median time to reach 100 μg/mL, which has been suggested to be the threshold for the onset of hemodynamic effects, was about 8 minutes for AQST-109 at a 4-minute residence time and 10 minutes for the 0.3 mg IM injection.

The current study demonstrated maximum plasma concentration (Cmax) values for the three residence times that were consistent with, 0.3 mg IM Injection, as well as those previously reported for approved injectable epinephrine devices such as EpiPen®.

This study confirms the ability to deliver significant levels of epinephrine through the oral mucosa, including a Tmax of 15 minutes or faster in multiple studies, which is particularly significant in the treatment of anaphylaxis.

### Example 30

The purpose of this investigation was to continue to study the administration of the film under a variety of conditions and further characterize its pharmacokinetics, pharmacodynamics, and safety. The final two arms of the study were designed to assess the impact of (1) administering the film sublingually two minutes after consuming food—in this case, a peanut butter sandwich, and (2) swallowing the film whole immediately with 240 mL of water. The control arm used for reference was the optimized sublingual film administration with a four-minute residence time.

Referring to FIG. 30 and as shown below, the data showed that administering the film sublingually two minutes after consuming a peanut butter sandwich had no statistical impact on the resulting pharmacokinetics when compared to the optimized sublingual film administration with a four-minute residence time from the initial Part 3 dataset. The medium time to maximum concentration (Tmax) and partial area under the curve (AUC) results were comparable and the time to reach Tmax remained 12 minutes whether sublingually administered after consumption of a peanut butter sandwich or not.

Tmax, partial AUC's, t½, and clearance were different from the optimized sublingual administration control group and confirmed that the sublingual absorption as separate and distinct from the gastrointestinal absorption. However, the data also showed that, although slower to Tmax, after swallowing the film whole with water, absorption was comparable to when the film was administered sublingually in terms of the mean maximal concentration (Cmax) and AUC.

| | 4-minute | After Peanut | Swallowed |
|---|---|---|---|
| | residence | Butter Sandwich | Film |
| Parameter | (N = 22) | (N = 19) | (N = 20) |
| Arithmetic | 678 (176) | 478 (117) | 524 (88) |
| Cₘₐₓ(pg/mL) | | | |
| (% CV) | | | |
| Geometric | 350 | 286 | 313 |
| Cₘₐₓ(pg/mL) | | | |
| AUC_{0-10 min}(hr*pg/mL) | 12.8 | 15.0 | 2.4 |
| AUC_{0-20 min}(hr*pg/mL) | 51.2 | 48.8 | 28.1 |
| AUC_{0-30 min}(hr*pg/mL) | 79.1 | 64.7 | 73.2 |
| AUC₀₋ₜ(hr*pg/mL) | 411.5 | 341.7 | 481.0 |
| Tₘₐₓ(minutes) | 12 | 12 | 25 |

| | 4-minute | After Peanut | Swallowed |
|---|---|---|---|
| | residence | Butter Sandwich | Film |
| Parameter | (N = 22) | (N = 19) | (N = 20) |
| t½(hr) | 1.72 | 1.74 | 3.08 |
| Kel(1/hr) | 0.403 | 0.398 | 0.225 |
| CL/F(L/hr) | 23666 | 29610 | 13469 |

Surprisingly, unlike conventional films, where a subject may not eat or drink for at least 15 mins (for sublingual films) and for 1 hr (for buccal films) prior and/or after administration, the data shows that the compositions herein are effective without food (direct swallow with water), or within 2 minutes after food consumption.

### Example 31

The study compared single doses of AQST-109 to EpiPen® 0.3 mg and epi 0.3 mg IM injection as well as repeat doses of AQST-109 to repeat doses of epi 0.3 mg IM injection. Results from the single dose administration showed AQST-109 achieved significantly faster Tmax (12 minutes) compared to both EpiPen® (22 minutes) and epinephrine 0.3 mg TI injection (45 minutes). The topline results of the study are shown in FIGS. 31A-D.

Referring to FIGS. 31A and 31B, these show that Cmax values within the timeframe critical to abate the cascade of anaphylaxis is comparable to, and well bracketed by the 0.3 mg IM and the EpiPen®. Similarly, referring to FIG. 31C, the AUC is comparable to the 0.3 mg IM and the EpiPen®.

Referring to FIG. 31D, AQST-109 showed significantly faster Tmax than EpiPen® in head-to-head comparison median time to maximum concentration (Tmax) of 12 minutes was faster than EpiPen Tmax of 22.5 minutes. AQST-109 repeat dosing (10 minutes after initial dose) provided significantly higher drug plasma concentrations when compared to a single dose while achieving a Tmax of 18 minutes. Changes in systolic blood pressure and heart rate were similar after a single dose of AQST-109 when compared to a single dose of EpiPen.

Referring to FIG. 31E, AQST-109 showed the fasted median Tmax compared to both 0.3 mg IM and the 0.3 mg EpiPen® Example 32

Referring to FIG. 32, a comparison is shown in terms of time to 100 μg/mL within 15 minutes. 100 μg/mL has been suggested as the onset of hemodynamic effects resulting from epinephrine exposure in the systemic circulation. AQST-109 provides a higher percentage of subjects reaching 100 μg/mL than 0.3 mg IM in this study. While 100% of subjects receiving EpiPen® reached 100 μg/mL within 15 minutes in this study, this is in contrast to previously reported EpiPen data Example 33

Referring to FIG. 33A and FIG. 33B, the pharmacodynamics results are shown comparins ghte response for systolic blood pressure (SBP) and pulse. The AQST-109 was more consistent with the EpiPen than with the 0.3 mg IM. Importantly, the single dose of AQST-109 showed similar or better changes in heart rate and systolic blood pressure when compared to the single dose of EpiPen. After one dose of AQST-109, maximum mean effects on systolic blood pressure occurred within 5 minutes of dosing compared to 8 minutes for EpiPen. Maximum mean effects in heart rate occurred within 8 minutes of administering AQST-109 compared to an average of 5 minutes within administering EpiPen. Safety results for AQST-109 were in line with expectations, and no severe or serious adverse events were reported.

### Example 34

Referring to FIG. 34, the repeat dose topline results are shown. The results from the repeat administration show all subjects receiving a second dose of AQST-109 received additional epinephrine. This is a critical proof point for the product. Plasma drug levels show rapid absorption of the 2nd dose with a Tmax of 20 minutes (8 minutes post second dose administration).

When a second dose of AQST-109 was administered ten minutes after the first dose, the median Tmax remained rapid at 18 minutes while extensive absorption was observed. The maximum concentration, or CMax, of AQST-109 was 465 μg/mL after one dose and 2,958 pg/mL after two doses. In comparison, the epi 0.3 mg IM injection CMax was 489 μg/mL after one dose and 911 μg/mL after two doses. The single dose of EpiPen resulted in a CMax of 869 μg/mL.

| | AQST-109 | 0.3 mg IM |
|---|---|---|
| Description | Repeat Dose | Repeat Dose |
| # Subjects/#Dosings | 23/24 | 23/24 |
| Cmax (pg/ml) | 2134 | 755 |
| (ISCV %) | | |
| AUC 0-t (hr*pg/ml) | 1469 | 1300 |
| AUC 0-30 min (hr*pg/ml) | 402 | 67 |
| Tmax (min) | 18 | 50 |
| Tmax Range (min) | 8-30 | 30-70 |

### Example 35

Referring to FIG. 35, a repeat dosing post-hoc analysis shows that subjects with high Cmax levels were significantly above therapeutic thresholds and mean. These subjects would be unlikely to require a second dose. For those with lower Cmax levels, a repeat dose increased drug levels adequately, but not excessively. The speed of the AQST-109 will allow both clinicians and patients to be informed about the need for a second dose while limiting unnecessary exposure.

AQST-109 has proven to be safe and well-tolerated in both single and repeat administrations. The results show that AQST-109's single dose safety profile had a lower incidence of palpitations than that of the 0.3 mg EpiPen®. AQST-109's repeat dose safety profile had a similar incidence of palpitations to that of the single administration of 0.3 mg EpiPen®. Local tolerability of both single and repeat administrations of AQST-109 remains good with adverse events that are mild to moderate and self resolving.

| | AQ109201 | | | | |
|---|---|---|---|---|---|
| | 12 mg | 24 mg | 0.3 mg | 0.6 mg | EpiPen |
| MedDRA | (N = 23) | (N = 23) | (N = 23) | (N = 23) | (N = 22) |
| SOC PT | n (%) | n (%) | n (%) | n (%) | n (%) |
| Cardiac | 2 (8.7) | 9 (39.1) | 1 (4.3) | 1 (4.3) | 7 (31.8) |
| disorders | | | | | |
| Palpitations | 2 (8.7) | 9 (39.1) | 1 (4.3) | 1 (4.3) | 7 (31.8) |
| Ventricular | 0 | 0 | 0 | 1 (4.3) | 0 |
| extrasystoles | | | | | |

While repeat epinephrine doses are infrequently needed, the data provided herein show a second administration of AQST-109 can rapidly boost plasma levels and bolster the magnitude of response to one that could occasionally be needed to stabilize anaphylaxis in the field. A repeat dose of AQST-109 delivered peak plasma levels after just 8 minutes, comparing favorably with the repeat-dose profile of the referenced standard product delivered in the same time period. The ability to quickly deliver an effective second administration, when and if needed, is an extremely positive attribute—particularly in a life-threatening condition.

Epinephrine can stop an avalanche leading to disaster but must be administered early—during a window of opportunity. Minutes and seconds are important as timely delivery opens airways and shores up the cardiovascular system, including leaky capillaries responsible for early swelling and angioedema. Epinephrine also has inhibitory effects on mast cells, direct effectors of an allergic response (and responsible for histamine release). Unfortunately, as each second goes by and anaphylaxis can progresses; the magnitude and extent become more difficult to reverse. Although administration in the field can still buy lifesaving time, stabilizing the patient until more sophisticated medical care is available is what then becomes most important. One key is how fast epinephrine gets into the bloodstream and to affected organs and tissues—only then can epinephrine exert palliative effects throughout the body. While allergic individuals are encouraged to carry around epinephrine syringes for direct injection, the reality is that majority of patients do not carry their auto-injector. A clear unmet need exists for a new administration method that enables both fast delivery to the bloodstream and is truly portable.

Referring to FIG. 31A and FIG. 31B, the box and whisker plots on the left that the Cmax produced by AQST-109 within 15 minutes of dosing is higher than the 0.3 mg injection and comparable to that of the EpiPen. The levels produced by AQST-109 are well bracketed by both injectable products suggesting efficacy on the low end and safety on the high end. The profiles on the right show that, with the exception of the 5-minute time point, the profiles for AQST-109 and EpiPen are virtually indiscernible. Also, in this profile the impact of repeat dosing of both AQST-109 and the 0.3 IM can be seen. While the repeat administration of the IM dose form has virtually no impact on the PK profile for over 20 minutes after second dose administration, AQST-109 is rapidly absorbed providing additional epinephrine within 2 min of second dose administration. The second dose provided peak plasma levels just 8 minutes later.

FIG. 33A and FIG. 33B show pharmacodynamics, specifically the Systolic Blood Pressure and Heart Rate. Both are expected to go up in response to epinephrine exposure, and both are used as surrogates for therapeutic effects of epinephrine to the cardiovascular system as well as the respiratory system through adrenergic receptor activation. The speed of AQST-109 is evident here as the SBP rapidly increases after administration. EpiPen is seen to have similar responses for both measures while the more slowly absorbing IM dose form has a lag in both responses. Interestingly, the EpiPen did not produce greater PD responses despite a higher overall higher Cmax compared to AQST-109 suggesting, at least hemodynamically, in certain circumstances, there is no measurable benefit to having a higher Cmax.

Referring to FIG. 34 and another important outcome of the study, which was the confirmation of the ability to deliver a second dose. With IM injections, local vasoconstriction in the tissue requires the second dose to be administered in a new location to ensure adequate effect. Given sublingual administration would be repeat dosed at the same location, it is critical to ensure the second dose can be administered and absorbed effectively. The study showed unequivocally that for every subject in the study, the second dose is rapidly and effectively absorbed. In fact, the second dose reached Tmax 8 minutes after second dose administration. That's faster than a single administration and still faster than a single administration of the EpiPen.

Referring to FIG. 35, the study was a controlled study in healthy volunteers. This means that a second dose was administered to every subject irrespective of what the plasma epinephrine levels and the magnitude of mechanism-based pharmacodynamics after the initial dose was given. This resulted in some very high plasma epinephrine levels. However, the need and use of a second dose can be quite rare. However, when a second dose is required, additional epinephrine the standard of care and as demonstrated in this study, irrespective of what your levels were at the time of second dosing, additional epinephrine was delivered. One reasonable perspective is that the need for a second dose is tied to those who may have had lower drug levels at the time of second dosing. Within this context, a post hoc analysis was performed. Here, individuals who were at epinephrine levels below 300 pg/mL, a very conservative number, were analysed specifically. For this subset, every subject responded to the second dose with increased plasma levels, but those levels were within the observed values of the repeat dose IM. And because AQST-109 has such a rapid Tmax, someone dosing a second time, at say 15 min, would likely be past Cmax, and if symptoms persisted, additional epinephrine is warranted. With the IM dose form, patients are still 30 minutes from Tmax, and while a second dose is the right choice for someone at that time to control symptoms, they still have a large and potentially unnecessary amount of epinephrine still to come. In this sense, AQST-109 may provide doctors and patients an alternative that avoids unnecessary exposure after symptoms have subsided.

AQST-109 continues to be safe and well tolerated, consistent with all studies to date. In comparison to the EpiPen, you will see that EpiPen had a much higher rate of palpitations (4-5×) than AQST-109. This is consistent with what is known about epinephrine, which is above a certain level AEs are increased but not necessarily effectiveness. Lastly, despite some of the very high exposure levels seen with the repeat dose of AQST-109, the rate of palpitations was only slightly higher than that of a single dose of epipen.

In summary, the Tmax of AQST-109 which as shown in the data had a faster Tmax than the EPIPEN, having levels that fall right between the 0.3 mg IM and the EpiPen bracketing safety and efficacy from the high and low sides respectively. The ability to deliver a second dose of AQST-109, and that the second dose is even faster than the first dose. Lastly, both single and repeat administrations are safe and well tolerated.

### Example 36

Studies described below include examining (1) differences in pharmacokinetic (PK) results based on changes to administration instructions, (2) additional repeat dose data on AQU-109 film, and (3) the differences between approved auto-injectors. The resulting data continue to show rapid absorption of epinephrine during the critical first ten minutes following administration of AQU-109 film.

As shown in FIG. 36, a pharmaceutical film of AQST-109 12 mg single dose PK study was completed in 12 healthy subjects with revised administration instructions. In this residence time study or “hold study,” the AQST-109 film was applied to the sublingual mucosa and held in place until dissolved (compared to the previous instructions which limited swallowing for 4 minutes following administration). The study resulted in a geometric mean Cmax of 400 μg/mL and a median Tmax of 10 minutes and a Tmax range of 5 minutes to 20 minutes. Importantly, AQST-109 film was significantly higher than Adrenalin 0.3 mg injection at all timepoints within the first 10 minutes following dosing. All but one of the subjects exceeded 100 μg/mL by 15 minutes following dosing. There were no significant adverse events during the study. This is the fastest median Tmax result to date for the AQST-109 film development program. AQU-109 film delivered epinephrine levels were significantly higher than epinephrine levels from the epinephrine 0.3 mg manual injection from previous study data, at all timepoints within the first 10 minutes following dosing. Similarity to approved auto-injectors during the first 10 minutes following administration is preferred. These results demonstrate meaningful improvements from previous administration instructions as the Company continues to optimize film administration.

Both blood pressure and heart rate were monitored during the study. Multiple pharmacodynamic markers were monitored including systolic blood pressure. A median change of 22 mmHg in systolic blood pressure was observed at 2 minutes following dosing with a significant change from baseline systolic blood pressure was maintained until 1 hour following dosing.

Also, AQST-109 film was safe and well-tolerated by subjects. There were no significant adverse events reported during the study and AQST-109 film continues to be safe and well-tolerated by subjects.

### Example 37

Given that manual epinephrine injections are rarely used outside of a clinical office setting, a pilot PK study was run to compare three different auto-injectors to the 0.3 mg manual injection (Belcher Pharmaceuticals). These auto-injectors were Auvi-Q (epinephrine) 0.3 mg auto-injector, EpiPen (epinephrine) 0.3 mg auto-injector, and the generic equivalent to EpiPen (Teva Pharmaceutical USA). These data will also be used to help identify the appropriate auto-injector(s) and PK values for comparing Anaphylm's performance in the upcoming pivotal study. A repeat dose study with subjects given a second dose 25 minutes after the initial dose. Importantly, minimal administration instructions were utilized potentially simulating non-compliance to Anaphylm's expected administration instructions. Consistent with another repeat dose study described herein, AQU-109 film produced a median Tmax of 8 minutes when re-administered after 25 minutes. Cmax and overall exposure were comparable to the auto-injectors demonstrating that a second dose of AQU-109 film can be used effectively at a later time period, as needed. There were no significant adverse events reported during any arms of this study.

Referring to FIGS. 37A and 37B, AQST-109 film 12 mg repeat-dose PK study in 27 healthy subjects conducted with limited dosing instructions. During this study, AQST-109 film was placed on the sublingual mucosa with no further restrictions. A second dose was administered at 25 minutes using the same dosing instructions. The geometric mean Cmax was 828 μg/mL with a median Tmax of 33 minutes, or 8 minutes following the second administration. The median change in systolic blood pressure following the second administration increased from 24 mmHg. A significant change from baseline systolic blood pressure was maintained until 2 hours following dosing. There were no significant adverse events during the study. The repeat administration at 25 minutes after the initial administration indicates that those in need of a second dose will receive the necessary epinephrine from AQST-109 film and that this can be done either very early, during the initial cascade, or as a booster later in the treatment cycle. Across two clinical studies using different administration instructions and at different administration timings; the repeat dose of AQST-109 film demonstrated consistent timing and proportionality of response.

### Example 38

Referring to FIGS. 38A, 38B and 38C, given that manual intramuscular (IM) injections such as Adrenalin are rarely used outside of a clinical setting, a pilot PK study was conducted to compare three different auto-injectors to the 0.3 mg manual IM injection. These auto-injectors were EpiPen, generic epinephrine auto-injector (Teva), and Auvi Q. The crossover study compared the results from 27 healthy subjects.

The studies can simplify AQU-109 film administration and can lead to optimized or eliminated residence times. For example, an AQU-109 sublingual film (12 mg) can be administered to achieve a 10-minute median time to maximum concentration (Tmax). PK was bracketed between available comparators for proposed pivotal study endpoints—both geometric mean maximum epinephrine concentration (Cmax) and partial area under the curve (pAUC). Safety profile was in line with previous studies—no severe or serious events were observed. With respect to auto-injector and 0.3 mg manual injection characterization, PK variability was established within and across available auto-injectors, data highlight similarities across auto-injectors and a target range (bracket) between IM and auto-injectors, and data can be utilized to statistically power the pivotal study. Endpoints included maximum plasma concentration (Cmax), time to maximum plasma concentration (Tmax), partial area under the curve at 10, 20, 30, and 45 minutes, and subjects reaching 100 pg/mL over time.

As shown in FIG. 38A, the geometric mean Cmax levels for EpiPen, generic epinephrine auto-injector, and Auvi Q were 628, 573, and 646 μg/mL, respectively, while the median Tmax times were 10, 15, and 30 minutes, respectively.

As shown in FIG. 38B, as a comparison, in the same study, the geometric mean Cmax level for Adrenalin 0.3 mg was 344 μg/mL with a median Tmax of 50 minutes.

FIGS. 38A and 38B show that the 12 mg median and mean Cmax levels are bracketed by the current FDA approved products and provides a consistently fast Tmax. Bars above show highest and lowest 75% quartile ranges of approved products.

As shown in FIG. 38C, the AQU-109 exposures are bracketed by those of auto-injectors and manual injection across a range of relevant times characterized as partial Area Under the Curve (pAUC). FIG. 38C depicts a comparison of AQST-109 film 12 mg to Combined Auto-injectors 0.3 mg and Adrenalin (epinephrine) Manual Injection 0.3 mg pAUC. The area under the curve (AUC) for the AQST-109 film provides exposures within the FDA aligned bracket through 45 minutes.

### Example 39

Referring to FIG. 39, this graph shows the geometric mean uncorrected epinephrine concentrations (pg/mL) over time in the first 10 minutes, specifically a comparison of epinephrine plasma concentrations over time of AQST-109 film (12 mg) to various approved auto-injectors (0.3 mg). The AQST-109 film geometric mean value achieved higher AUC within the first 10 minutes than the IM, and within a similar window between the IM and Epipen, comparable to Auvi-Q.

| | 0.3 mg | | | | |
|---|---|---|---|---|---|
| | IM | AQST-109 | EpiPen | Generic | Auvi-Q |
| Parameter | (n = 27) | (n = 12) | (n = 27) | (n = 29) | (n = 29) |
| AUC_{0-10 min} | 5.3 | 23.1 | 43.7 | 37.3 | 26.7 |
| (hr*pg/mL) | | | | | |

### Example 40

Referring to FIG. 40A, this shows a study comparing mean adjusted changes in systolic blood pressure (SBP) after administering the AQST-109 film compared with Epinephrine IM 0.3 mg (manual) and Epinephrine IM 0.3 mg (EpiPen).

Referring to FIG. 40B, this shows a study comparing mean adjusted changes in pulse (PR) after administering the AQST-109 film compared with Epinephrine IM 0.3 mg (manual) and Epinephrine IM 0.3 mg (EpiPen). The data was gathered from healthy volunteers, single period, n=12.

As indicated in the data, the autoinjectors produced rapid increase in systolic blood pressure (SBP) and Pulse and diastolic blood pressure (DBP) was observed within 2 minutes. The AQST-109 produced moderate increases in SBP and pulse with no measurable effect on DBP. The data was gathered from healthy volunteers, single period, n=12.

Referring to FIG. 40C, this shows a repeat dose study comparing the concentration (pg/mL) over time of a pharmaceutical film and 0.3 mg IM Epinephrine. This indicates a repeat dose of the AQST-109 film at 10 minutes and 25 minutes. The data was gathered from healthy volunteers, 6-period, n=24.

| | 0.3 mg IM | AQST-109 Film |
|---|---|---|
| | Repeat Dose | Repeat Dose |
| Description | (10 min) | (25 min) |
| # Subjects | 23 | 27 |
| Cmax (pg/mL) | 755 | 882 |
| AUC_{0-t min} | 1300 | 776 |
| (hr*pg/mL) | | |
| AUC_{0-45 min} | 181 | 207 |
| (hr*pg/mL) | | |
| Tmax (min) | 50 | 33 |
| Tmax (min-max) | 30-70 | 10-70 |

### Geometric Means Presented for Cmax, AUC0-t, AUC0-45. Median Tmax.

Data presented from cross-study analysis of 0.3 mg manual injection repeat dose at 10 min and AQST-109 film repeat dose at 25 minutes. The studies provide a target range between existing epinephrine injections provided by auto injectors, confirm Cmax comparability, and bracketing of pAUC parameters.

In summary, time to maximum blood concentration (median Tmax) for AQST-109 film was 10 minutes with a range of 5 to 20 minutes, early drug exposure at 10 minutes (partial area under the curve, or pAUC0-10 min) for AQST-109 film was similar to Auvi-Q® (epinephrine injection) auto-injector 0.3 mg and over 4 times higher than epinephrine 0.3 mg manual injection, while lower than both EpiPen® (epinephrine) auto-injector 0.3 mg and the generic equivalent product, and pharmacodynamic effects were observed as early as 2 minutes for both AQST-109 film and the auto-injectors.

### Example 41

Referring to FIG. 41, this shows an exemplary method of making a pharmaceutical composition. In an embodiment, a diisobutyryl epinephrine (DESF) dry film composition can have the following formulation. In an embodiment, the pharmaceutical film can include at least one adrenergic receptor interacter (e.g., DESF F5). In certain embodiments, more than one adrenergic receptor interacters can be added (e.g., DESF F10). In certain embodiments, two or three different adrenergic receptors for example, can be combined to modulate, alter or improve the pharmaceutical film's absorption profiles and organoleptic properties (e.g., DESF Formulations 284-1-1, 285-1-1 and 286-1-1):

| Material | Dry Film % w/w |
|---|---|
| Diisobutyryl epinephrine HCl | 25.00-30.00 |
| Pregelatinized hydroxypropyl pea starch | 1.000-19.000 |
| Polyvinylpyrrolidone | 21.000-24.000 |
| Polyethylene oxide | 1.000 |
| Silicon dioxide | 5.000-11.600 |
| Silicon dioxide (second) | 5.000-11.600 |
| Glycerol monooleate | 1.000 |
| Polysaccharide | 1.000 |
| 8-Hydroxy-p-cymene | 3.333-7.500 |
| Benzyl alcohol | 3.333-15.000 |
| Eugenol | 3.333-17.500 |
| Hydrochloric acid | 0-0.667 |
| Sweetener | 1.500-1.990 |
| Polacrilex resin | 1.000 |
| Sodium fluoride | 0.700 |
| Sugar alcohol | 0-0.500 |
| Edetate disodium, dihydrate | 1.000 |
| Surfactant | 1.000 |
| Mint flavor | 1.000 |
| Mixed berry flavor | 7.000-9.000 |
| Monoammonium glycyrrhizinate | 0-0.250 |
| Glycyrrhizic acid | 0-0.030 |
| FD&C Color | 0.010 |

Ester prodrugs can be prone to hydrolysis and loss of assay after storage was attributed to the presence of residual moisture in the films. In order to minimize the presence of moisture (free water) in this example, an absorbant component such as silicon dioxide was used. The absorbant component was found to reduce the hydrolysis degradation and result in a films with reduced tackiness.

The process depicted in FIG. 41A is an exemplary method of making a pharmaceutical film. The indicated times are approximate. FIG. 41A indicates that a process of making a pharmaceutical film can start with step 120, the addition of purified water and dye (e.g., FD&C Color) pregelatinized hydroxypropyl pea starch, polyethylene oxide, polysaccharide, sweetener, edetate disodium dihydrate, flavor, glycyrrhizic acid and hydrochloric acid and then mixing and homogenizing. In a separate container, in step 140A, silicon dioxide is pretreated with selected adrenergic receptor interacter(s) and mixed until dispersed for about 10 minutes or until dispersed as a dry powder. This separate submixture from step 140A is then combined with the mixture from step 120. Next, in step 150, ethyl alcohol is added, followed by water soluble polymer and mixed and homogenized until dispersed and hydrated.

In a separate container, in step 160A, diisobutyryl epinephrine HCl (DESF), labrasol, and ethyl alcohol are mixed and agitated until fully dissolved. Next, in step 160, this submixture is mixed and homogenized with the mixture of step 150 until it is dispersed. After degassing, in step 180, the mixture is then transferred to a coating line. Thereafter, the bulk coated film is unitized and packaged. In certain embodiments, each film unit dosage is weighed, and if within an acceptable range, each unit is packaged.

FIG. 41B depicts a comparison of individual adrenergic receptor interacters in terms of amount permeated as a function of time. In a comparison of adrenergic receptor interacters, cherry propanol has exhibited a low permeation that is concentration dependent (about 0-50 ug over 360 minutes), while benzyl alcohol was observed to have a mid-level permeation that is concentration dependent (about 50 ug-100 ug over 360 minutes), and eugenol was observed to have the most efficient permeation (about 50-275 ug).

FIG. 41C depicts ex vivo permeation as a function of time of different adrenergic receptor interacters in DESF compositions. The ex-vivo permeation in this example involved the exposure of porcine buccal tissue to formulations varying with regard to permeation enhancer(s) used and API concentration. Differences were observed in total API percentage permeated between test formulations and the DESF F5 formulation control. The DESF Formulation 285-1-1 showed a low total API percentage permeated regardless of increase in API concentration compared to the control, while surprisingly, DESF Formulations 284-1-1, 286-1-1 showed an increase in total API percentage permeated. Amount permeated increases with eugenol content, where eugenol contents in both test formulations were lower than that used in the control.

FIG. 41D shows prodrug flux as a function of time different adrenergic receptor interacters. Differences were observed in API flux between formulations DESF 284-1-1, 285-1-1, 286-1-1 and the DESF F5 control. Formulation 285-1-1 showed API flux regardless of increase in API concentration compared to the control. Surprisingly, formulations 284-1-1 and 286-1-1 showed an increase in API flux with increases in eugenol content, where eugenol contents in both test formulations were lower than that used in the control.

FIG. 41E shows amount permeated as % of total load as function of time with different adrenergic receptor interacters. Ex-vivo permeation involved the exposure of porcine buccal tissue to formulations varying with regard to permeation enhancer(s) used and API concentration. Differences were observed in total API percentage permeated between formulations DESF 284-1-1, 285-1-1, 286-1-1 and the DESF F5 control. Formulation 285-1-1 showed low total API percentage permeated regardless of increase in API concentration compared to the control. Surprisingly, formulations 284-1-1, 286-1-1 showed an increase in total API percentage permeated. Amount permeated increased with eugenol content, where eugenol contents in both test formulations were lower than that used in the control. In short, a more complete permeation was demonstrated for the 286-1-1 formulation compared to 284-1-1 and to the control.

Primary endpoints for epinephrine delivery by film had values bracketed between injectable products for (1) Cmax and (2) AUC₀₋₁₀ₘᵢₙ, AUC₀₋₂₀ₘᵢₙ, AUC₀₋₃₀ₘᵢₙ, and AUC₀₋₄₅ₘᵢₙ as follows:

Cmax Bracketing

| | Amnt (pg/mL) |
|---|---|
| Auvi-Q | 520.6 |
| Anaphylm | 470.2 |
| EpiPen | 469.2 |
| Adrenalin | 308.2 |

Partial AUCs Bracketing (hr*pg/mL)

| | AUC_{0-10 min} | AUC_{0-20 min} | AUC_{0-30 min} | AUC_{0-45 min} |
|---|---|---|---|---|
| Adrenalin | 3.3 | 11.3 | 29.7 | 74.6 |
| Anaphylm | 17.7 | 71.5 | 109.7 | 138.6 |
| EpiPen | 23.4 | 62.0 | 112.4 | 179.8 |
| Auvi-Q | 21.8 | 61.5 | 104.8 | 200.3 |

Referring to FIG. 42A, this shows the impact of administration in concentration as a function of time (0-60 minutes).

Referring to FIG. 42B, this shows the impact of administration in concentration as a function of time (0-20 minutes).

Referring to FIG. 43, this shows pharmacokinetic parameters by treatment with the pivotal dosing instructions (pharmacokinetic parameters compared to a manual hold).

Referring to FIG. 44, this shows the administration effect on low responders, indicating an improved proportion of subjects reaching 100 μg/mL threshold.

Referring to FIG. 45, this shows the results of a bridge study with concordant outcomes using the same administration conditions of the pharmaceutical film.

Referring to FIG. 46, this shows a geometric mean plasma profile as a comparison of epinephrine plasma concentrations (in mg/mL) 10 minutes after dosing of the claimed pharmaceutical film and various approved auto-injectors at 0.3 mg.

Referring to FIG. 47, this shows a pAUC comparison of the claimed pharmaceutical film in 14 mg (top yellow line) and 12 mg (lower yellow line) compared to autoinjectors (shaded regions), showing that longer exposure data supports a dose selection of 12 mg based on upper bracket excursion with a 14 mg dose).

Referring to FIG. 48A, this shows Cmax (pg/mL) for the claimed pharmaceutical film bracketed by current FDA approved products (bars showing highest and lowest 75% of approved products).

Referring to FIG. 48B, this shows Tmax (minutes) for the claimed pharmaceutical film bracketed by current FDA approved products (bars showing highest and lowest 75% of approved products).

Referring to FIG. 49A, this shows the increase in systolic blood pressure within 2 minutes (following administration) between the claimed pharmaceutical film and autoinjectors.

Referring to FIG. 49B, this shows the increase in pulse within 2 minutes (following administration) between the claimed pharmaceutical film and autoinjectors.

Referring to FIG. 50, this shows the results from a study of the pharmacokinetic bracketing at a 12 mg dose between adrenaline 0.3 mg and Epipen 0.3 mg in healthy subjects.

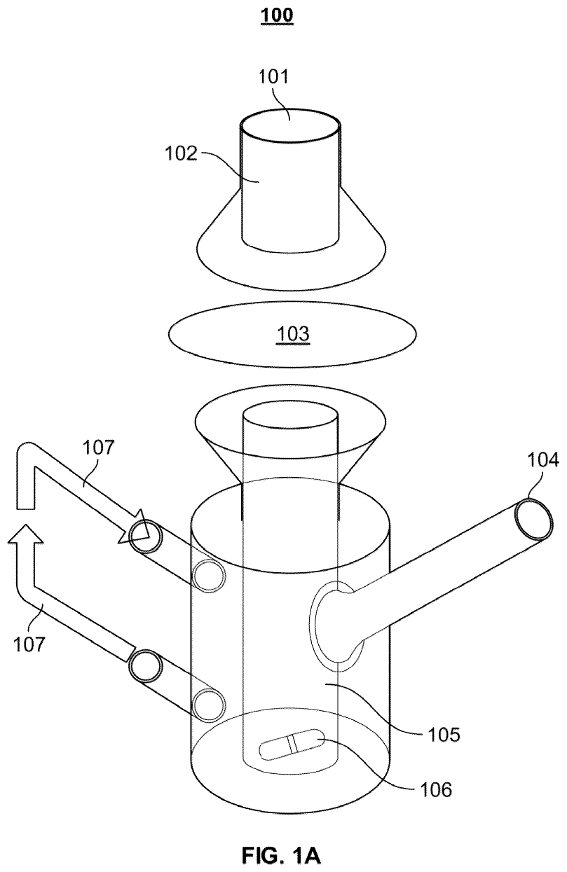

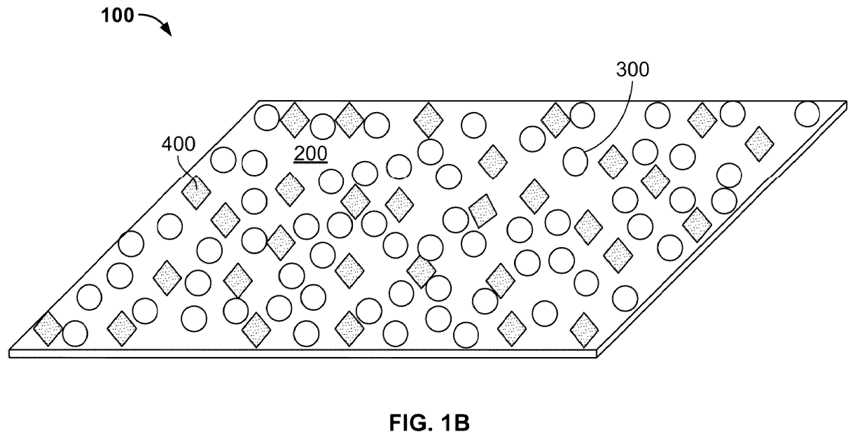

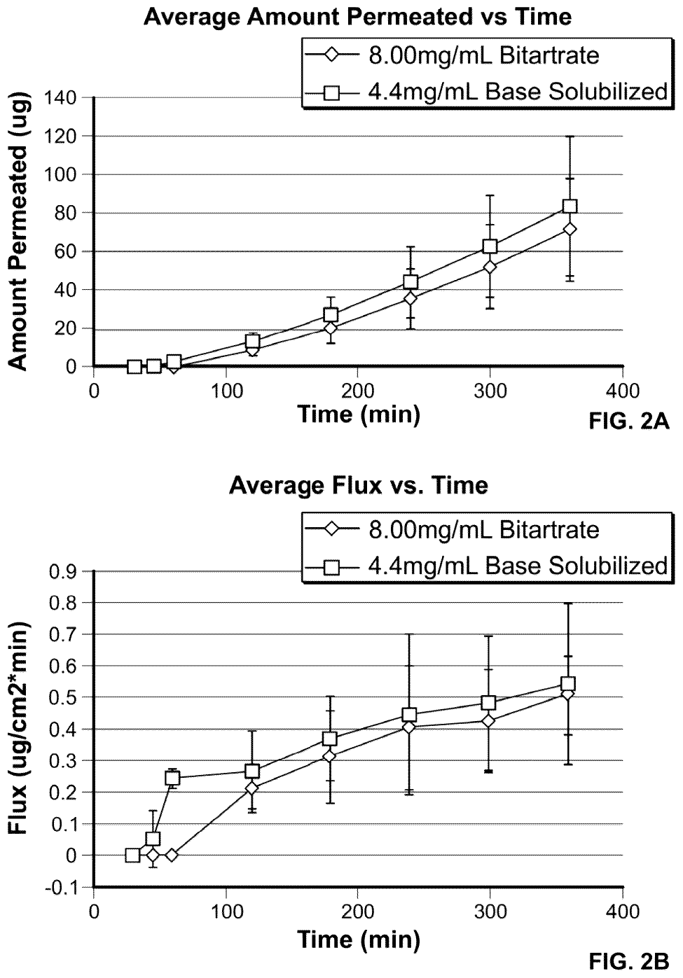

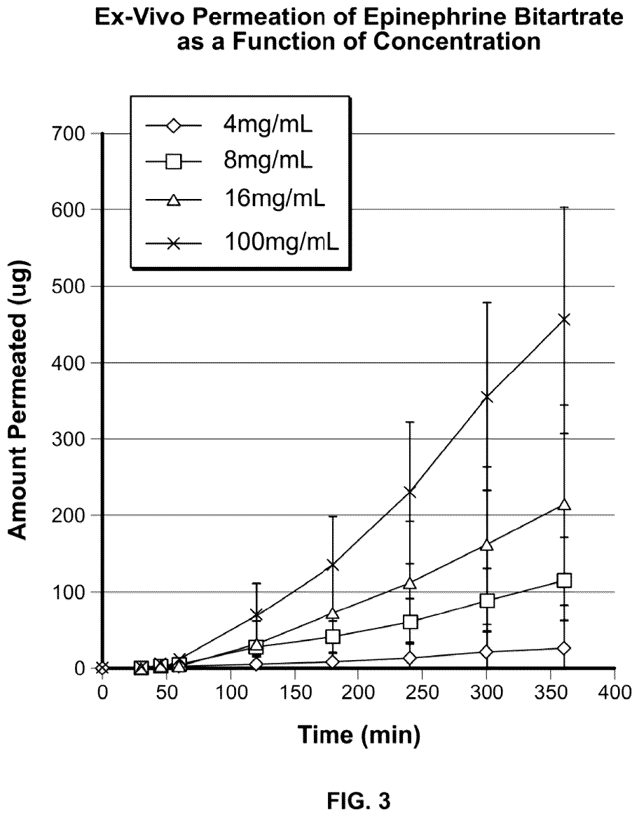

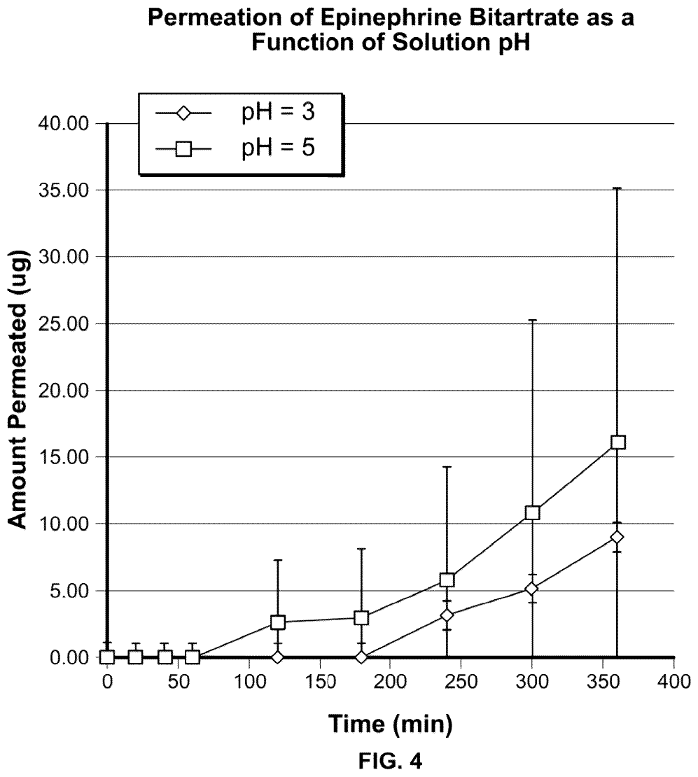

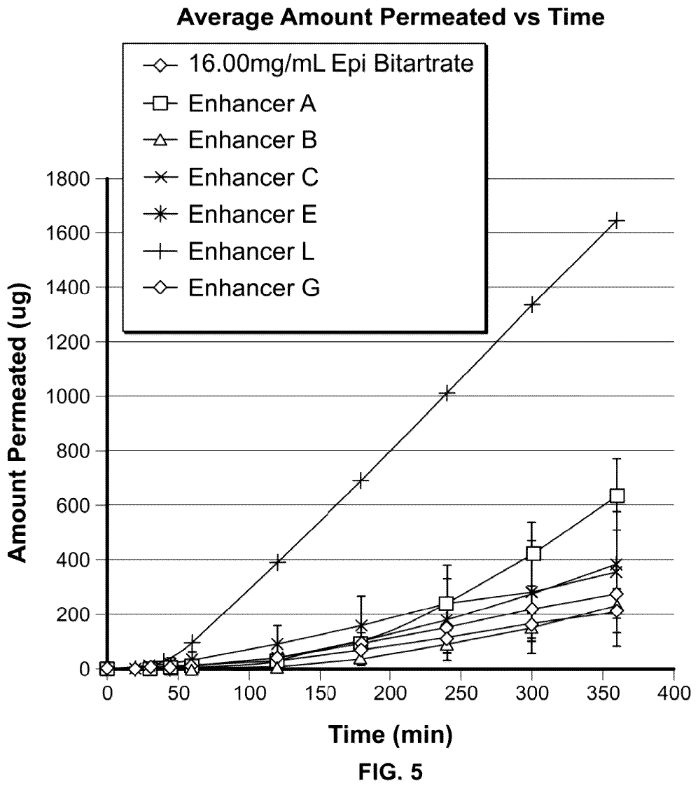

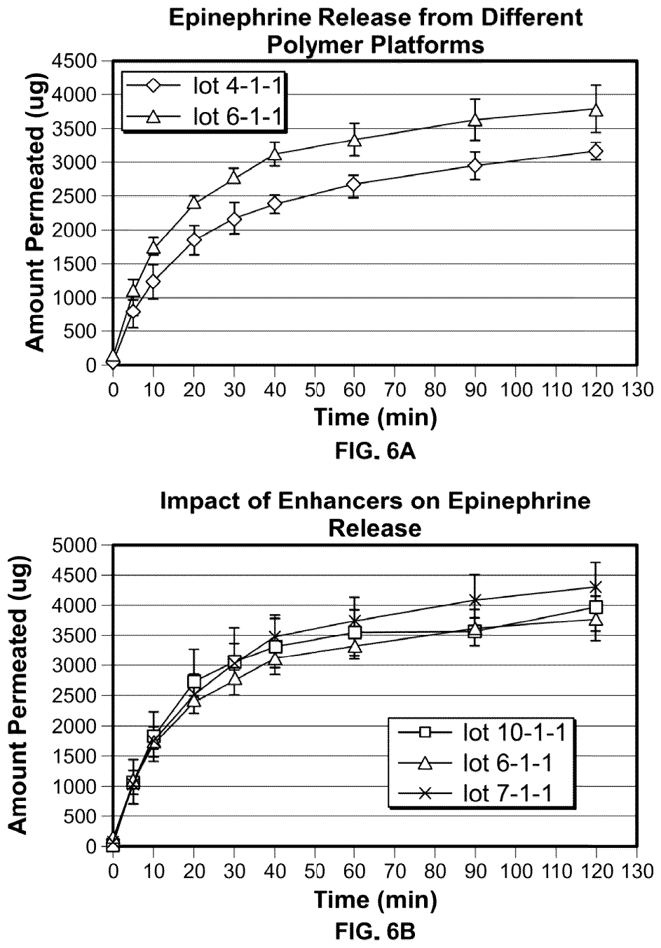

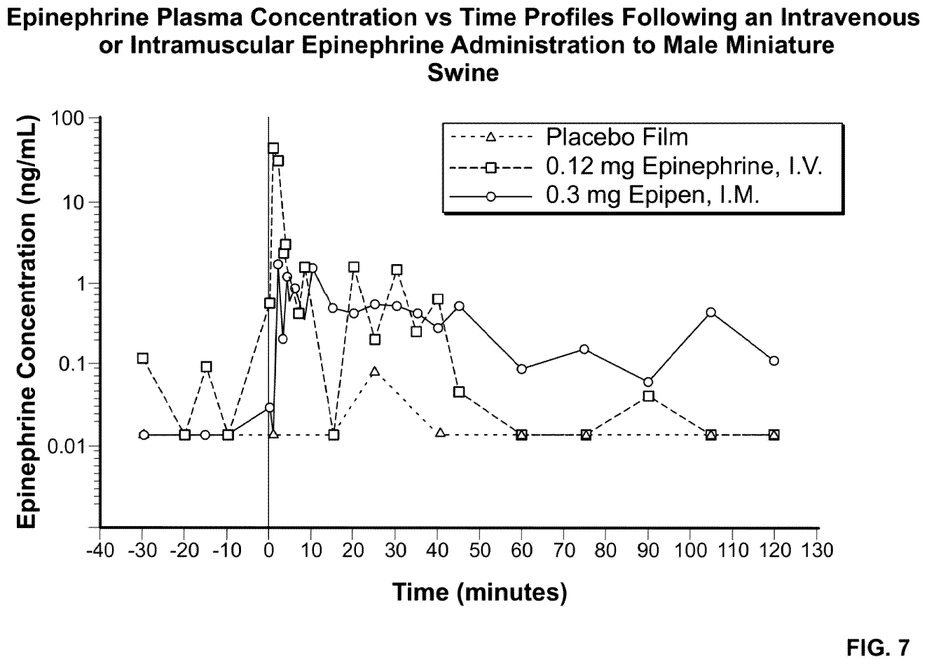

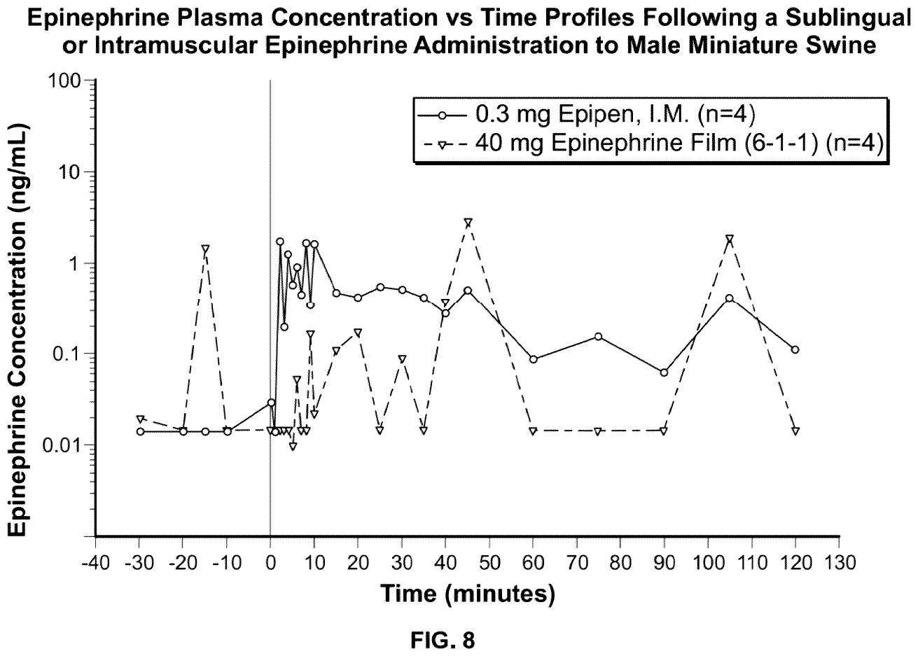

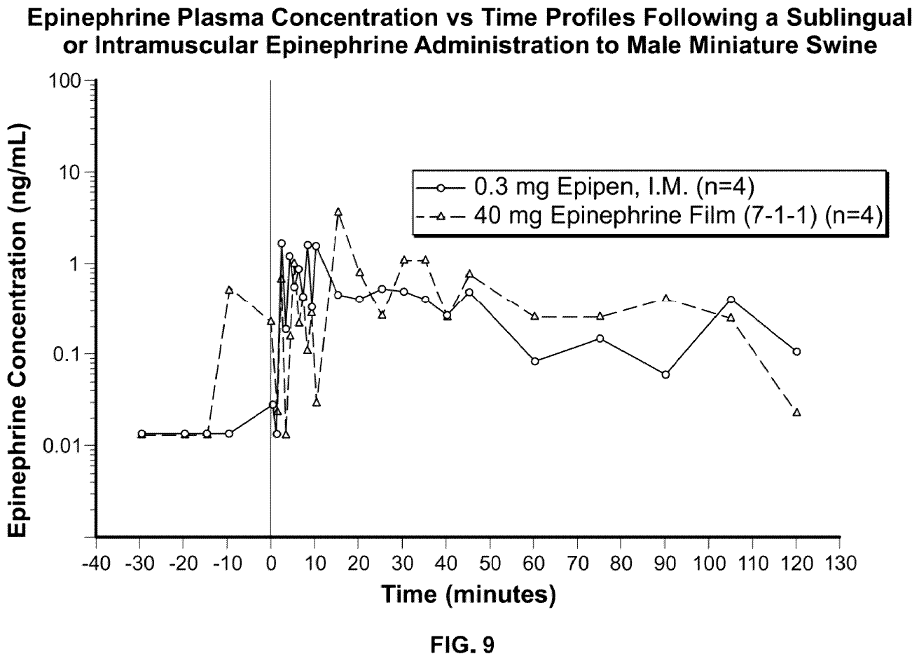

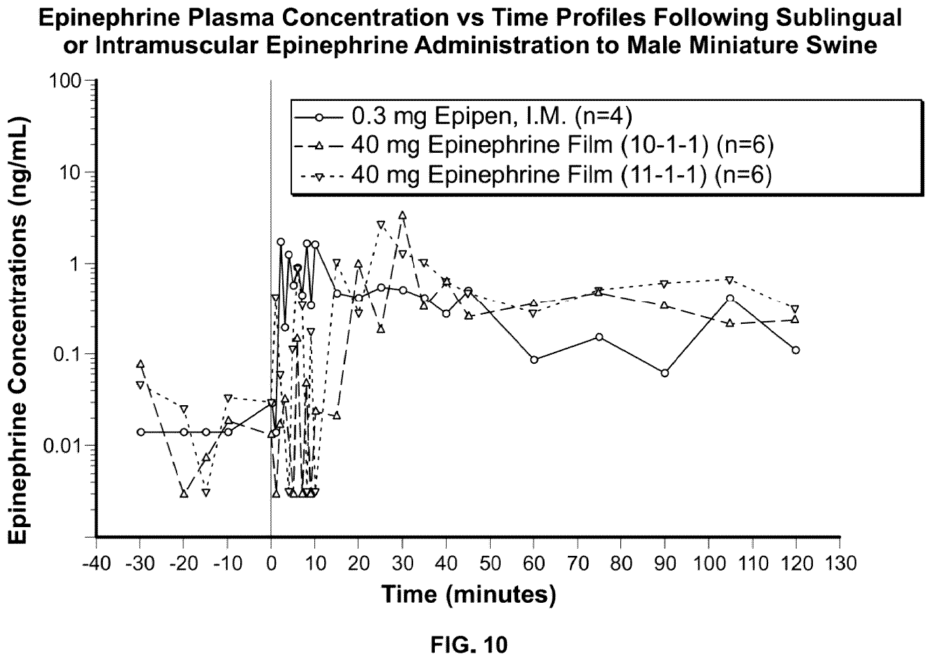

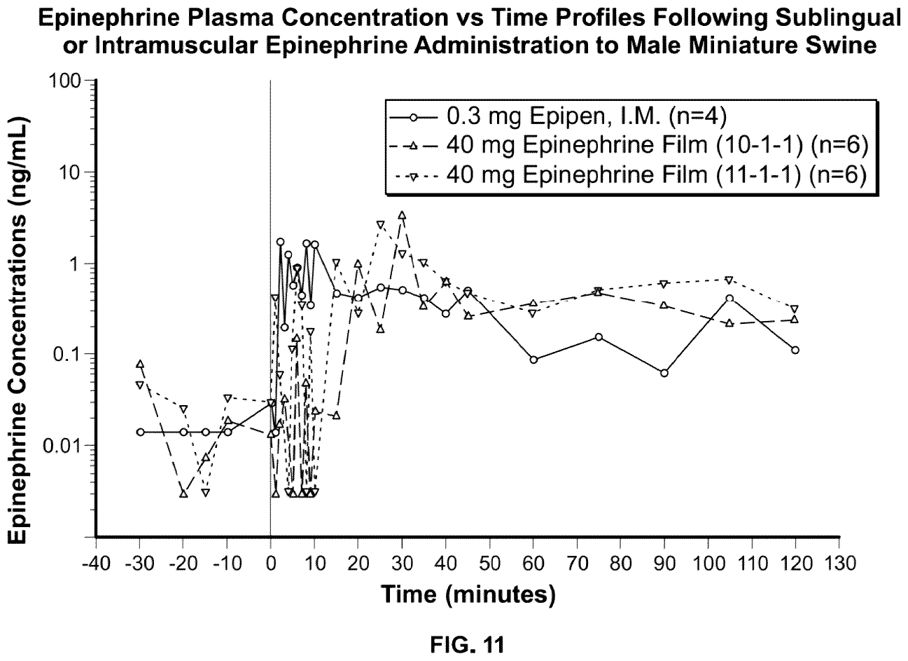

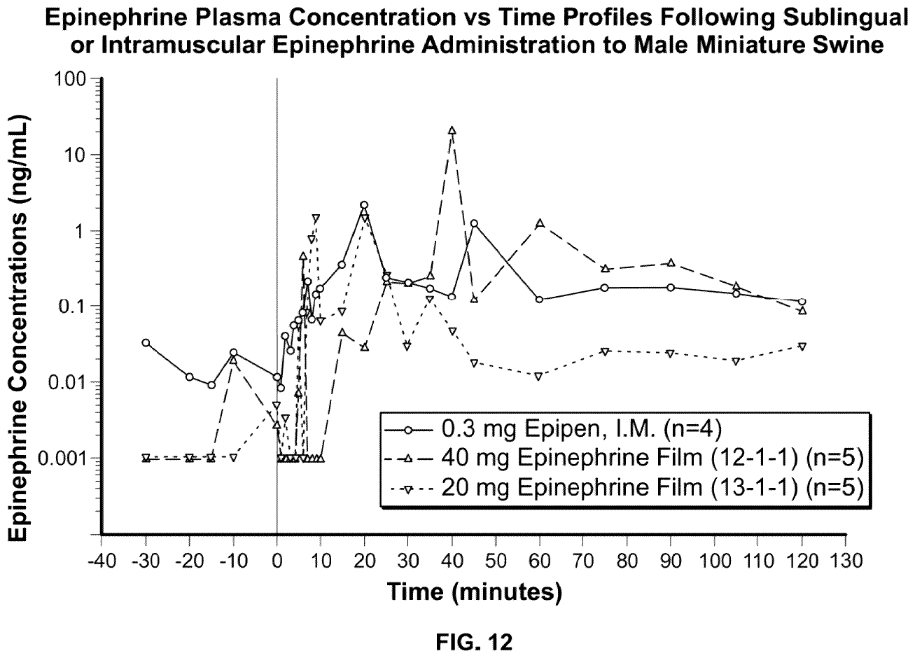

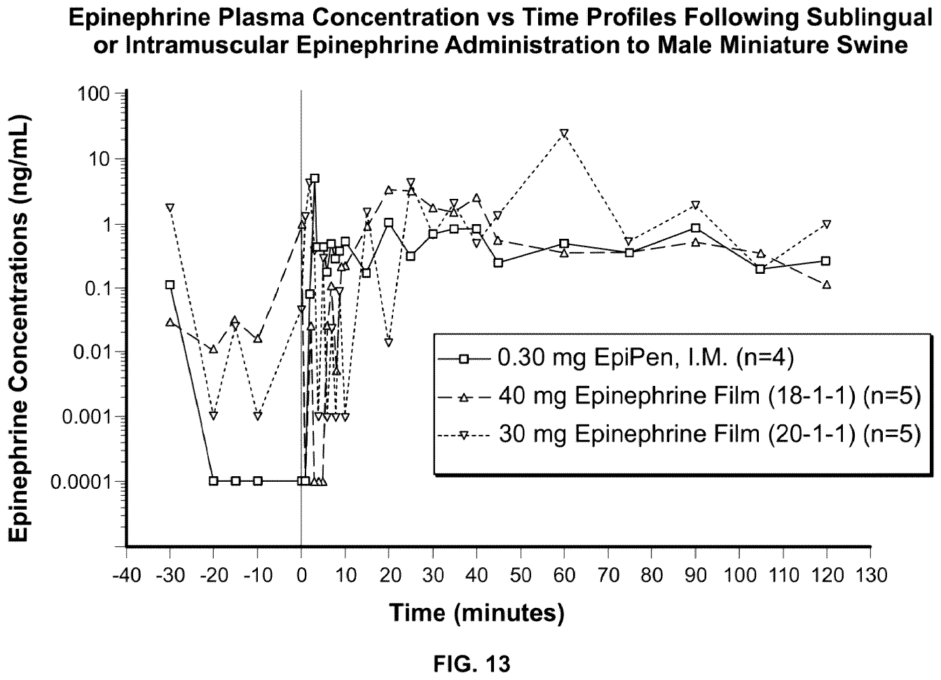

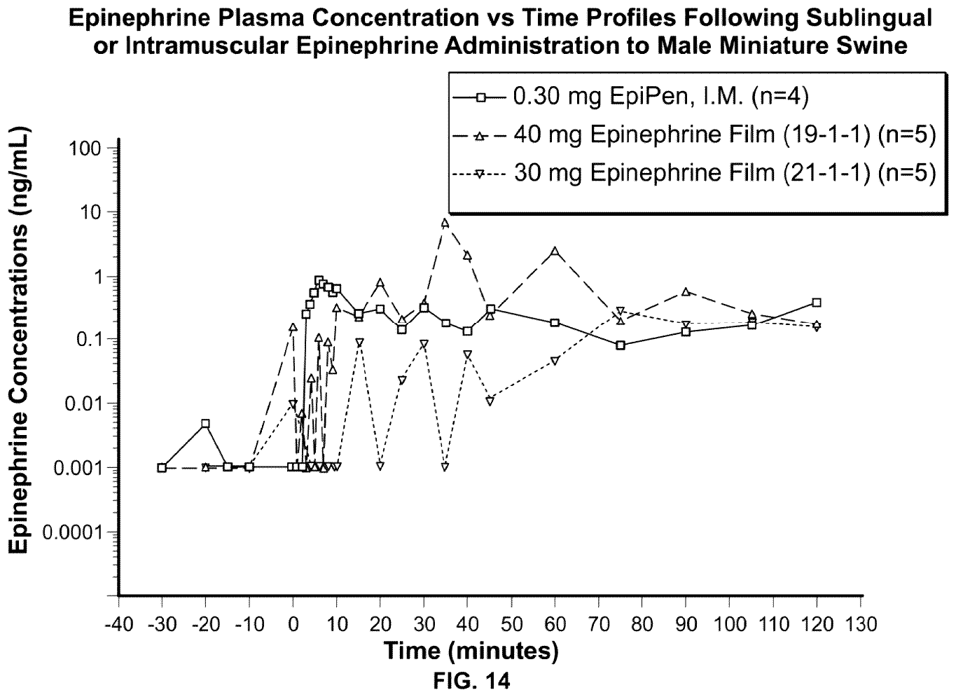

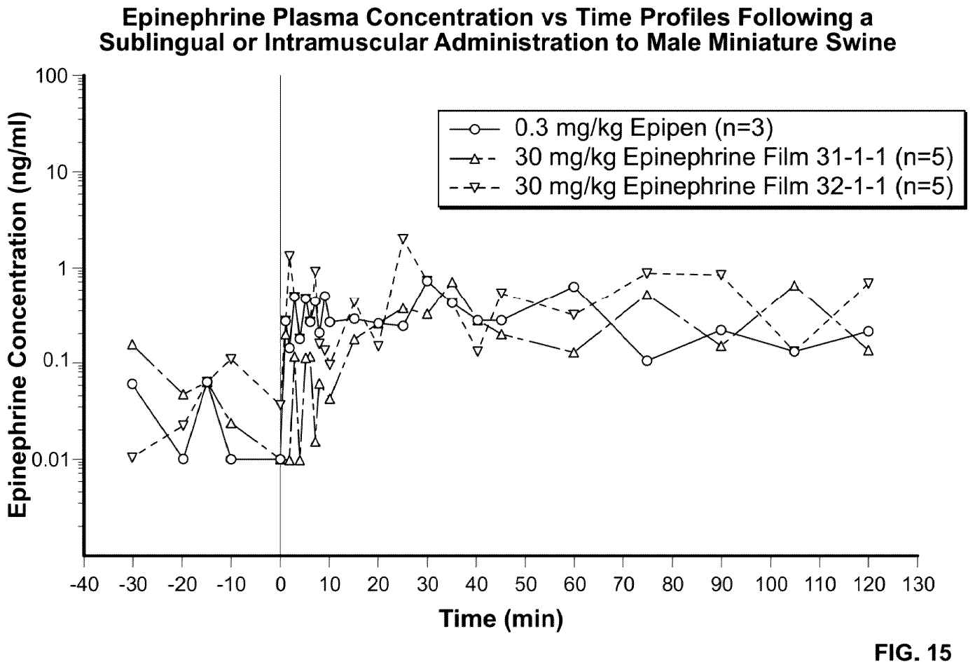

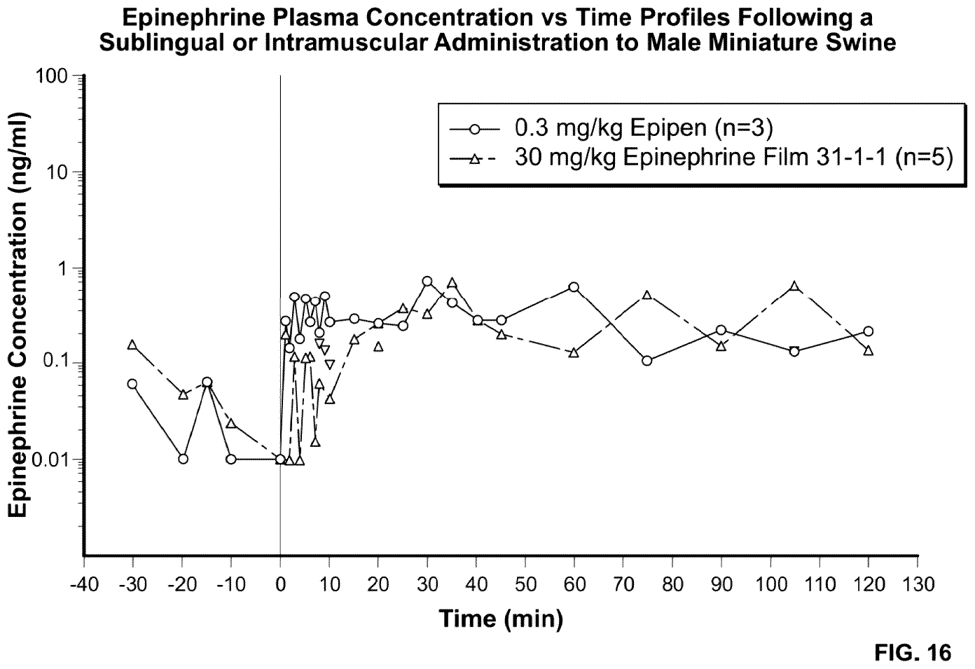

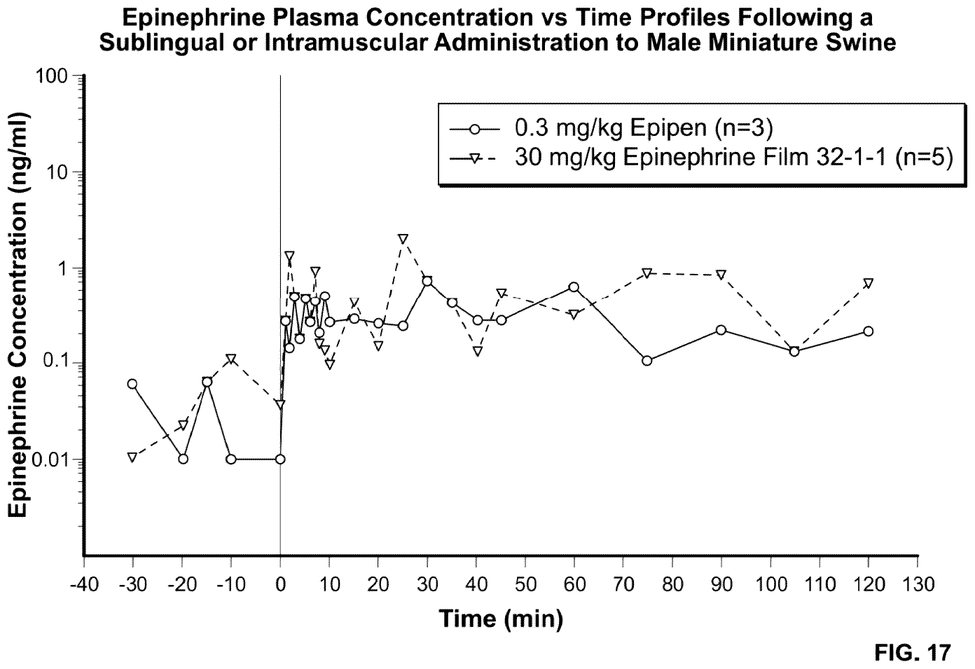

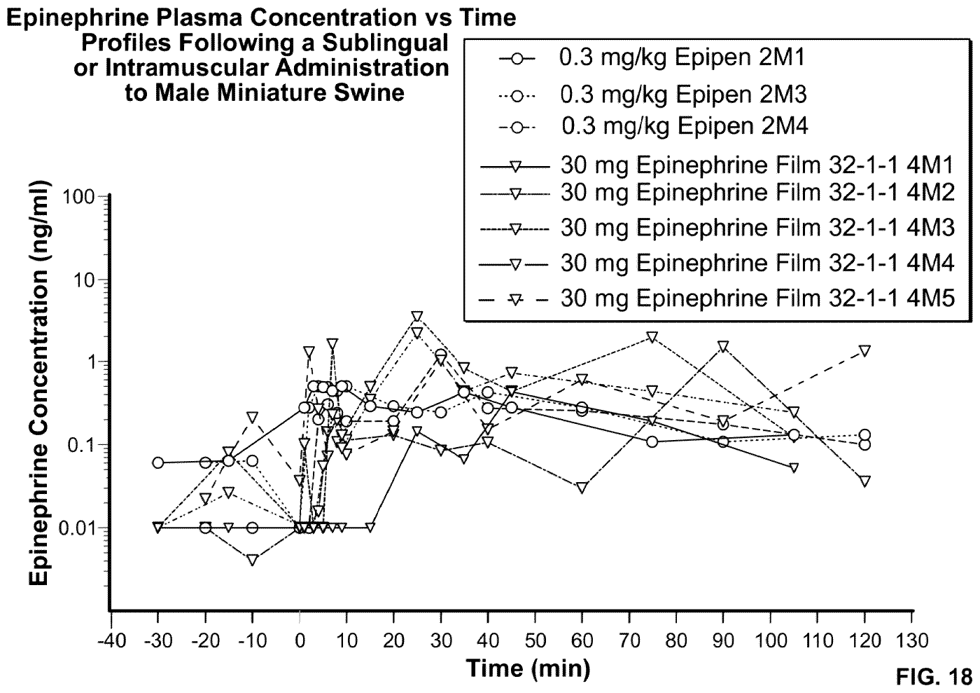

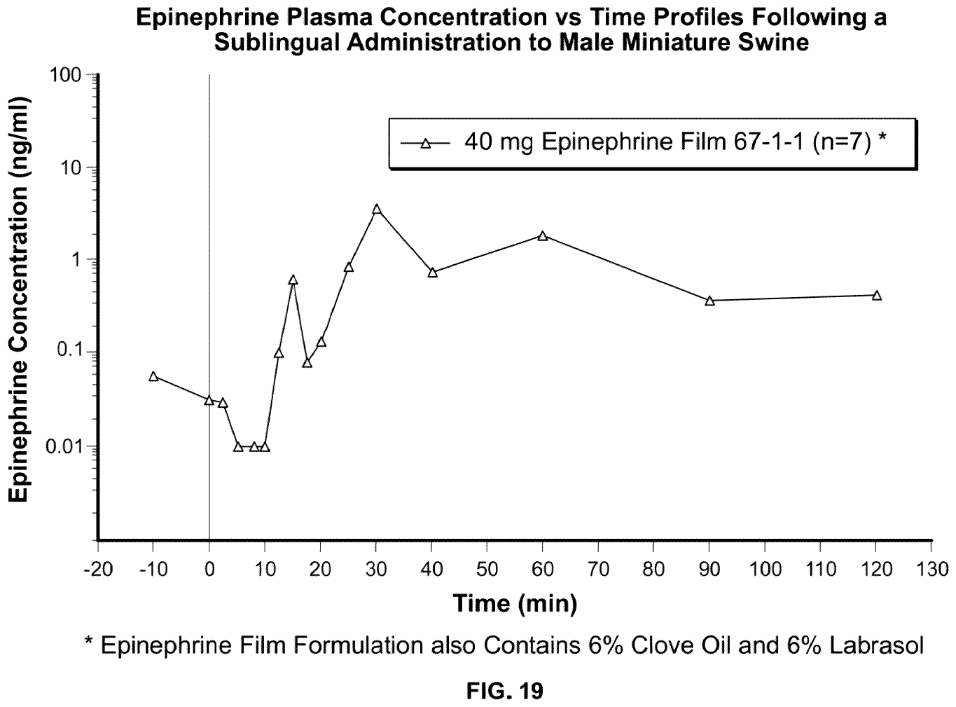

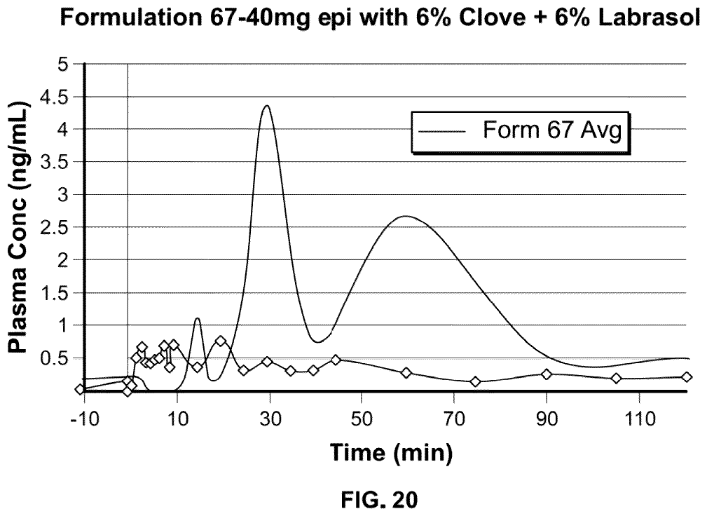

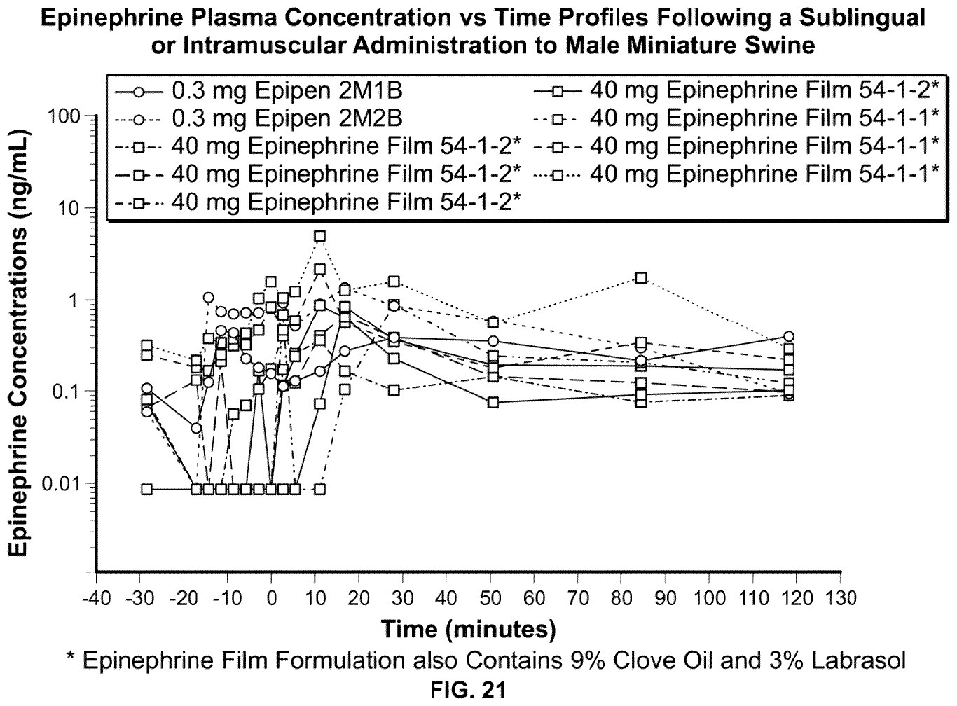

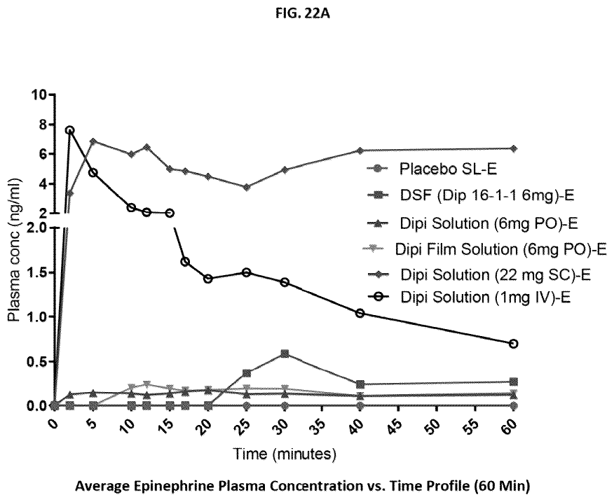

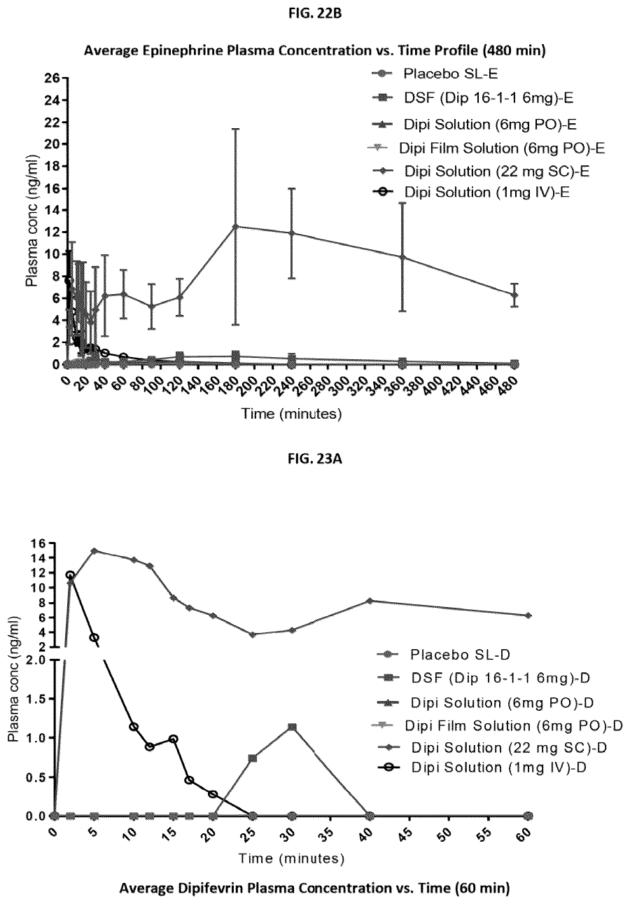

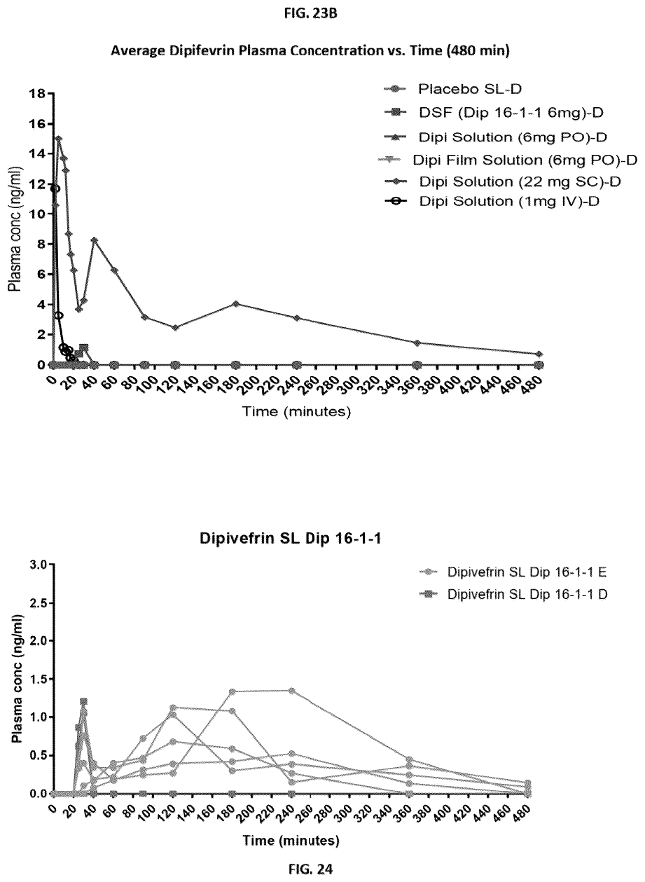

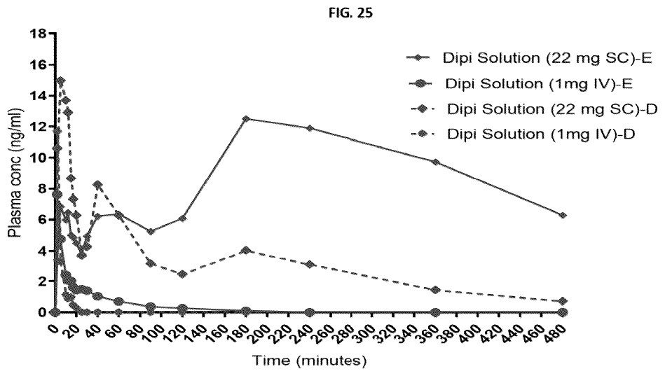

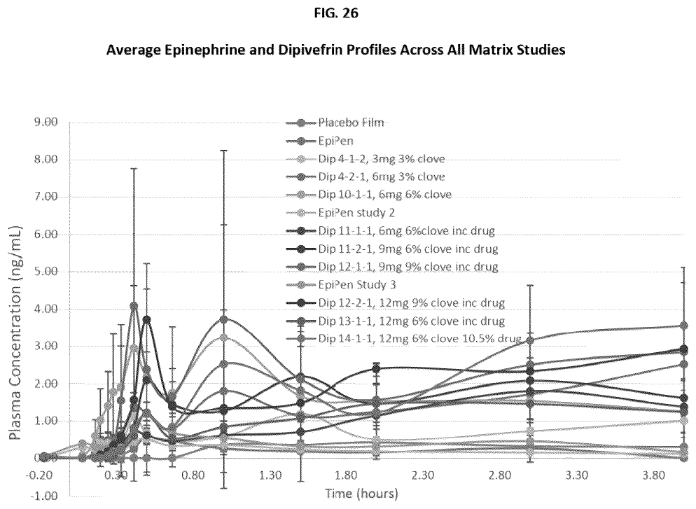

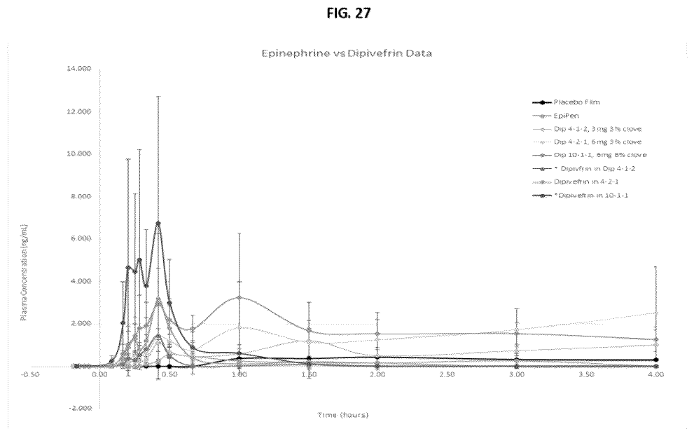

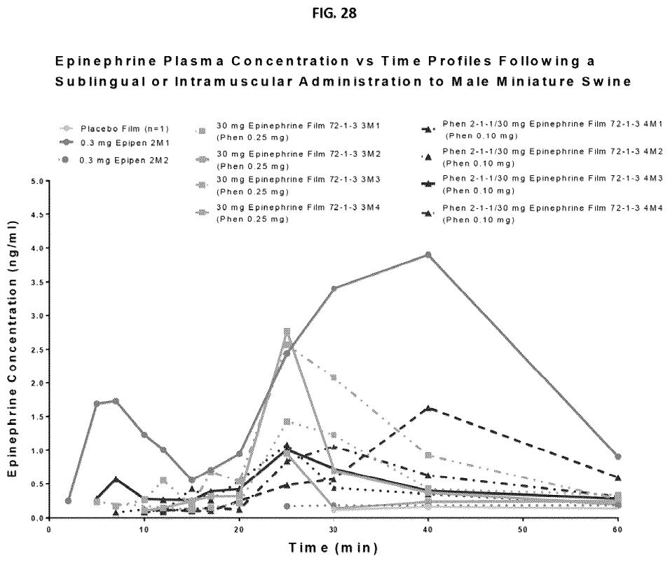

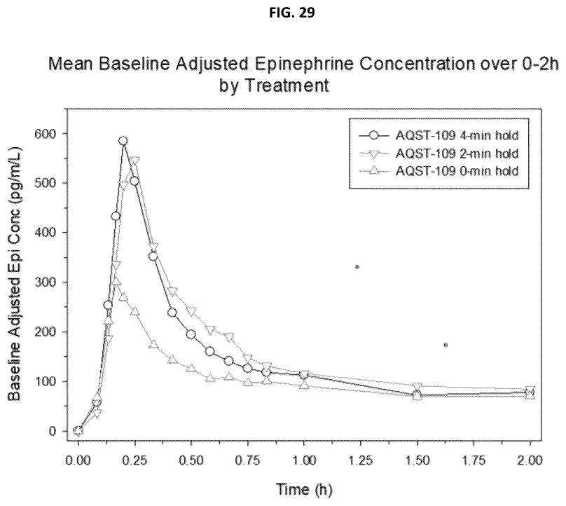

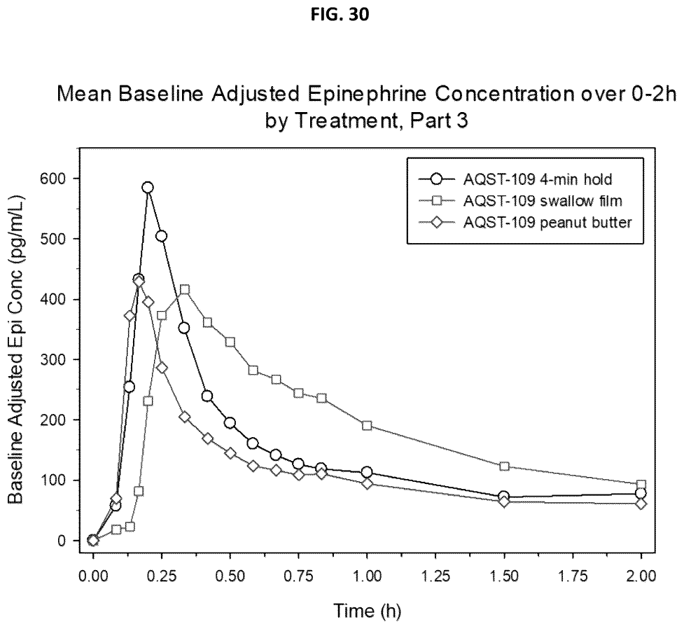

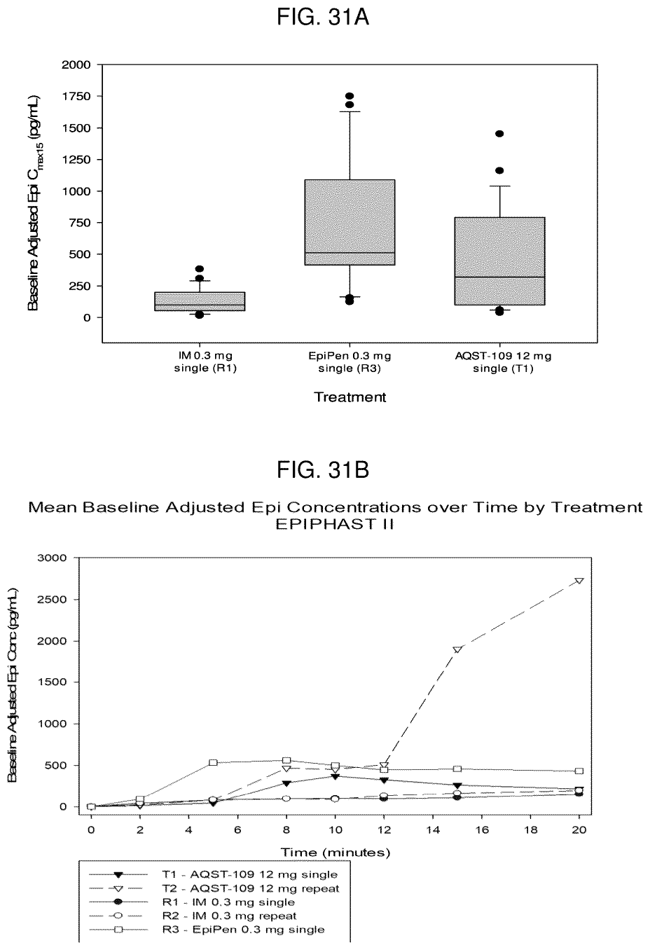

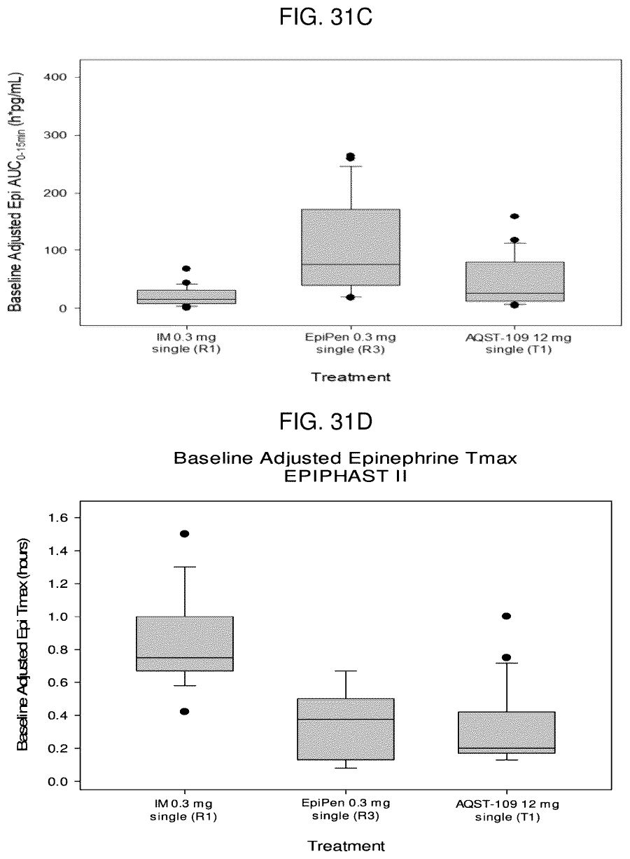

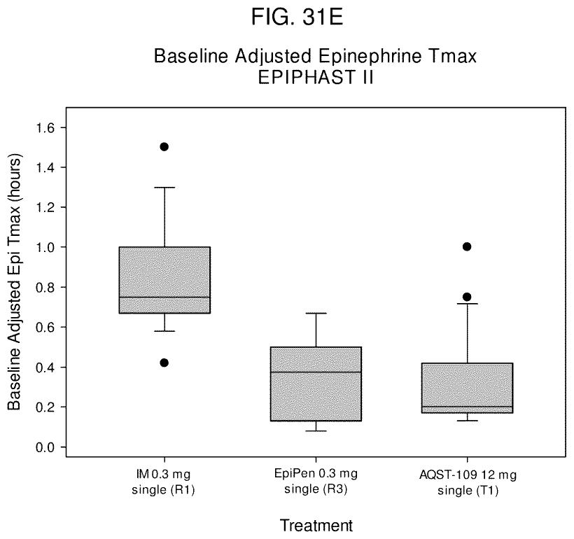

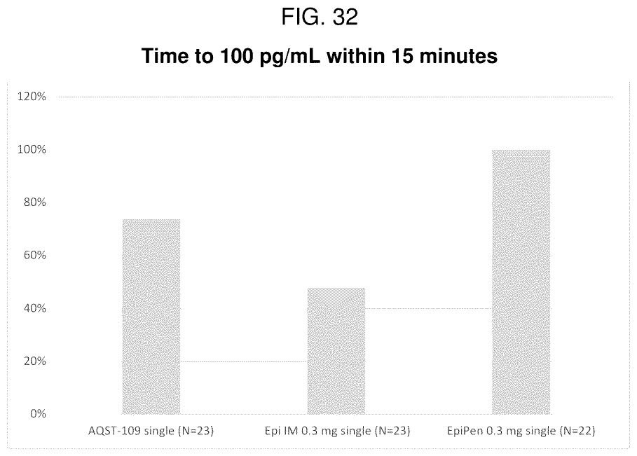

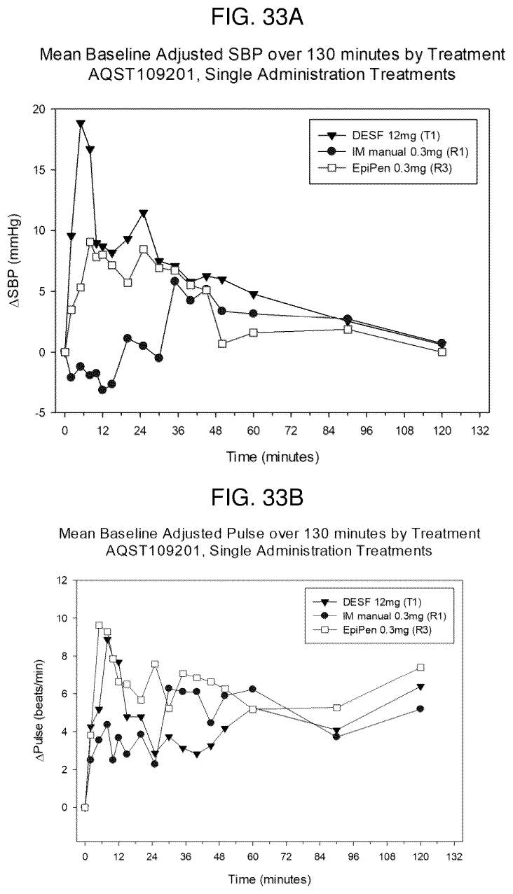

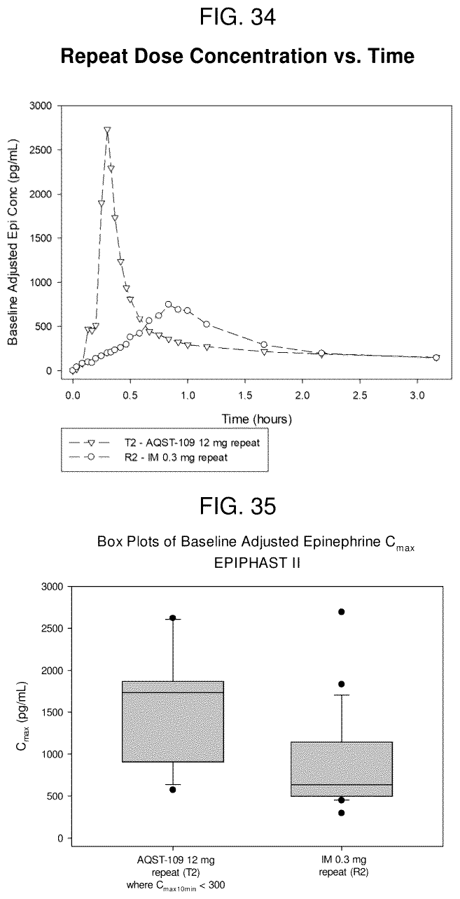

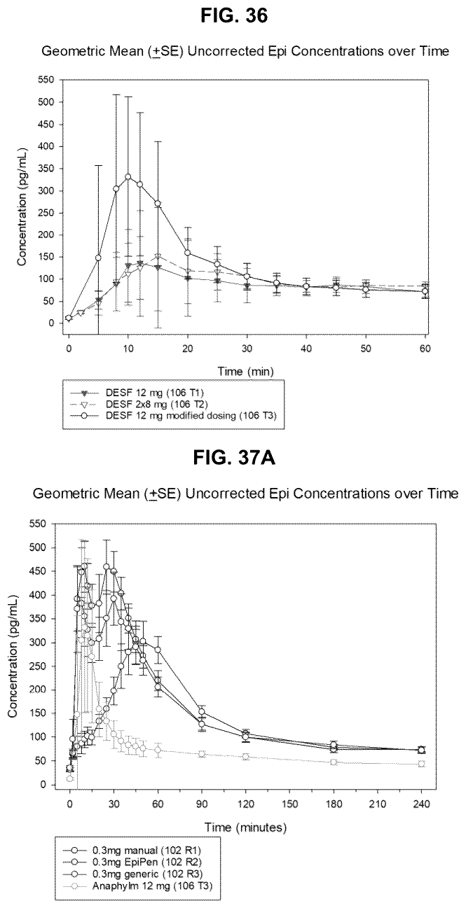

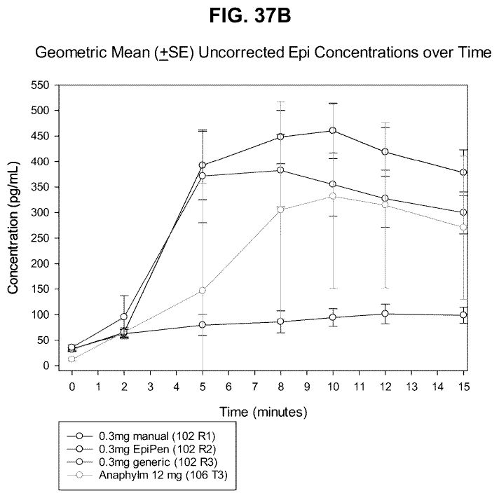

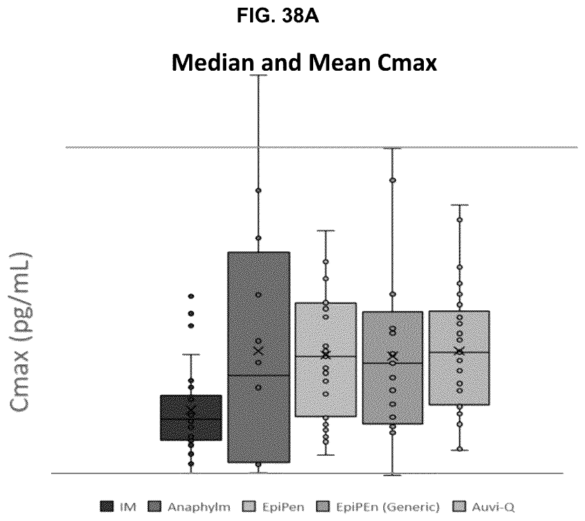

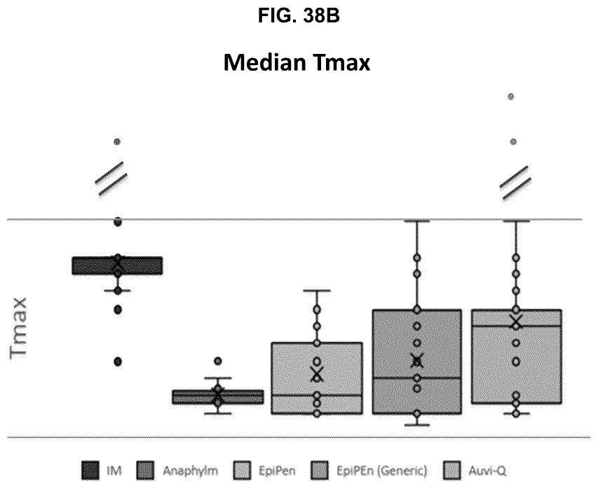

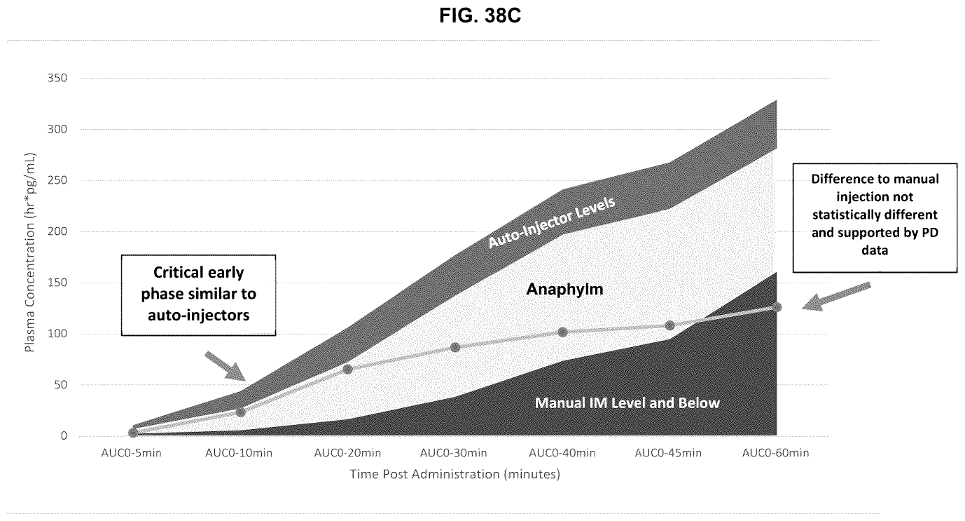

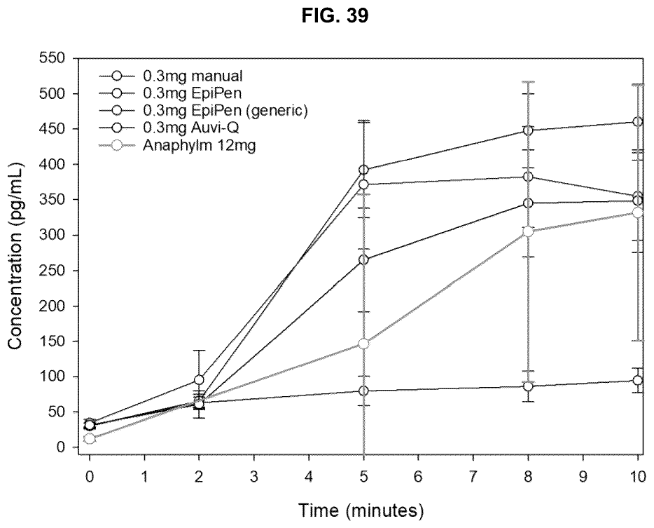

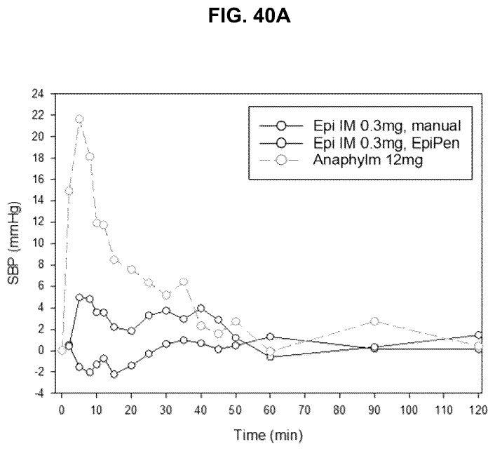

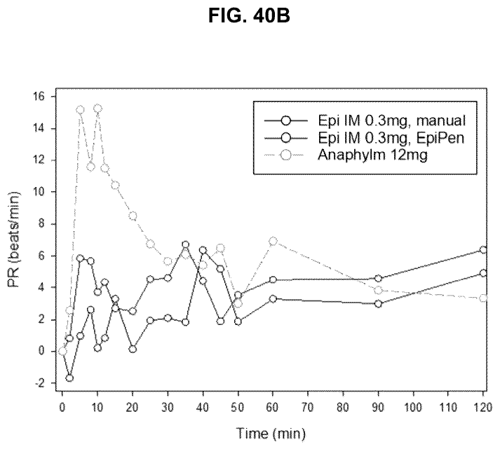

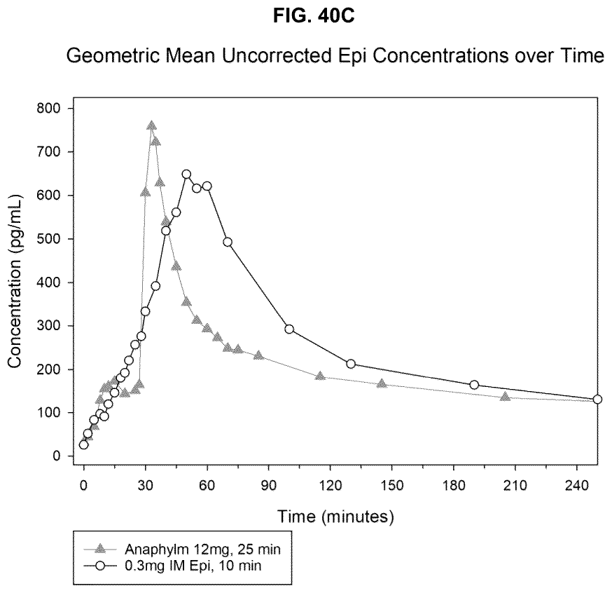

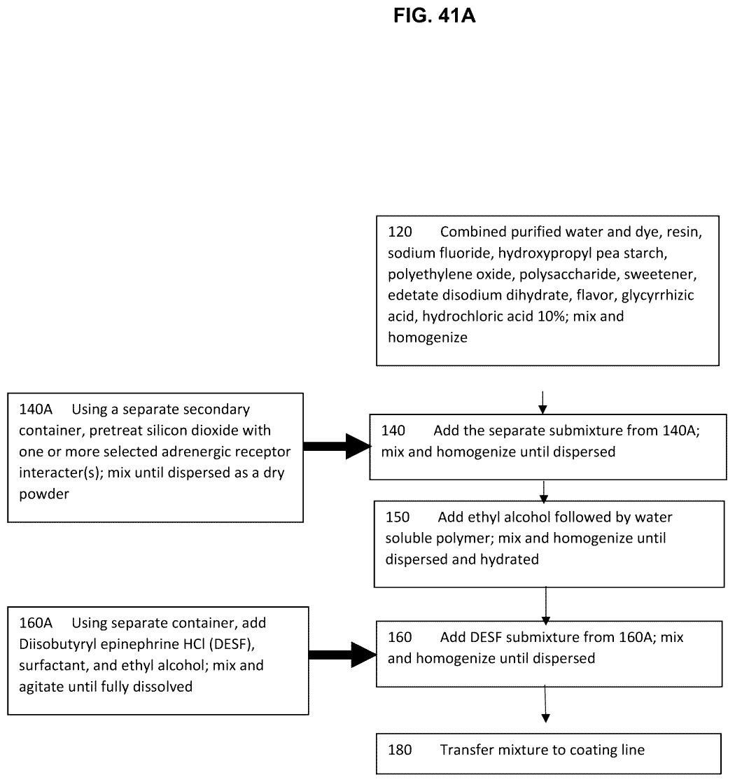

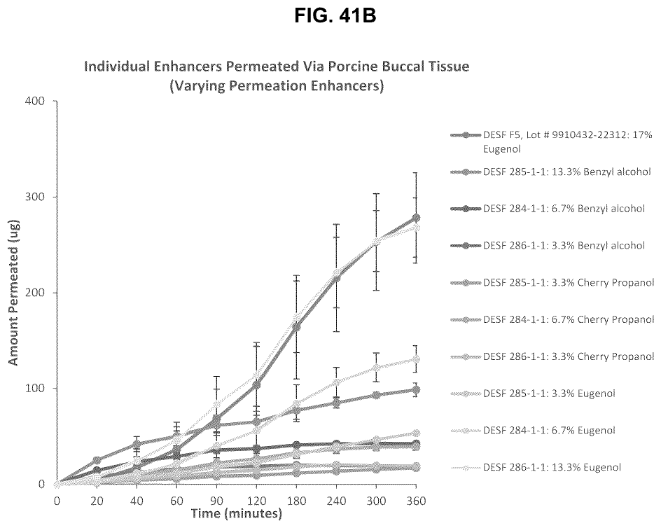

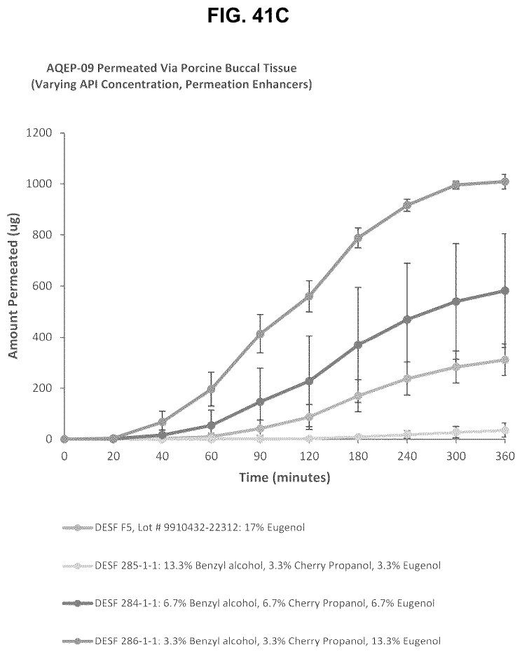

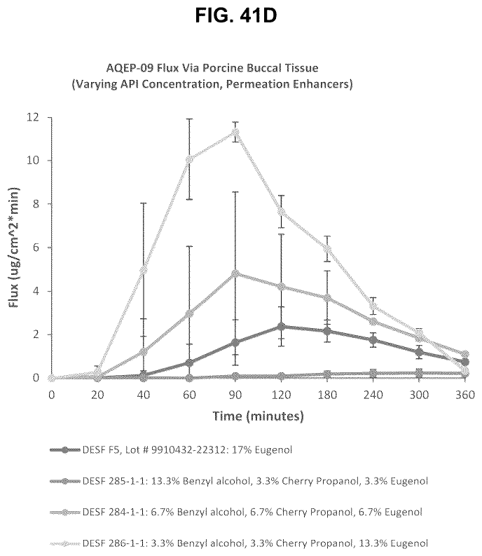

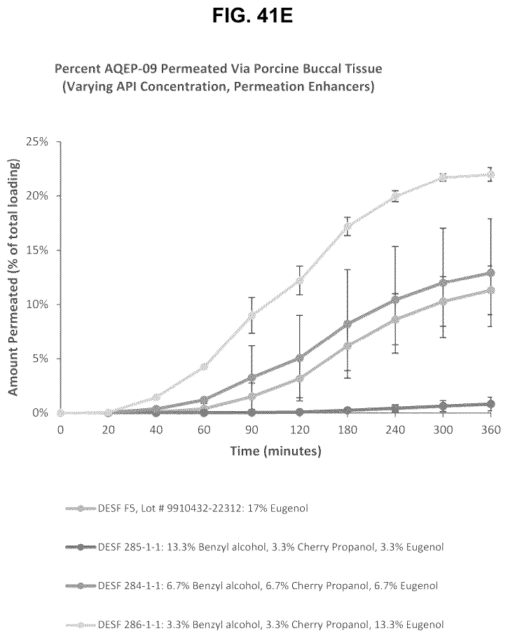

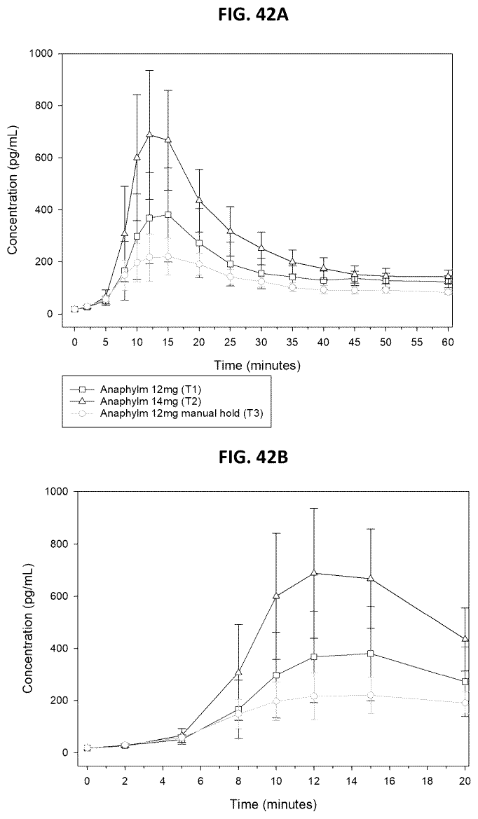

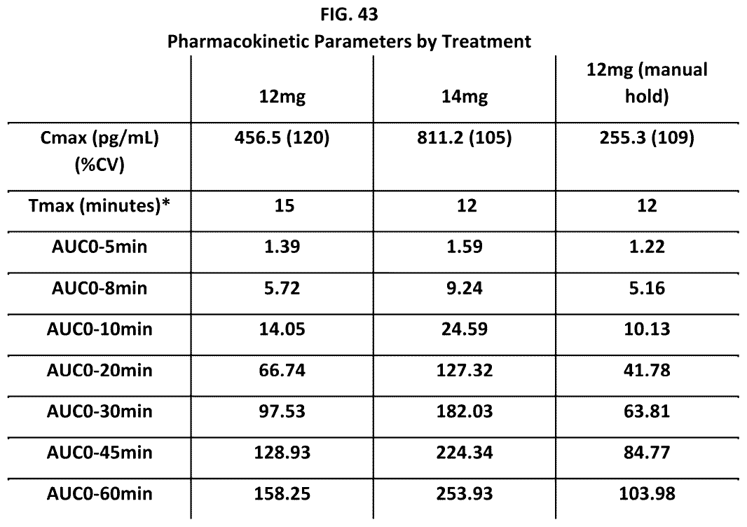

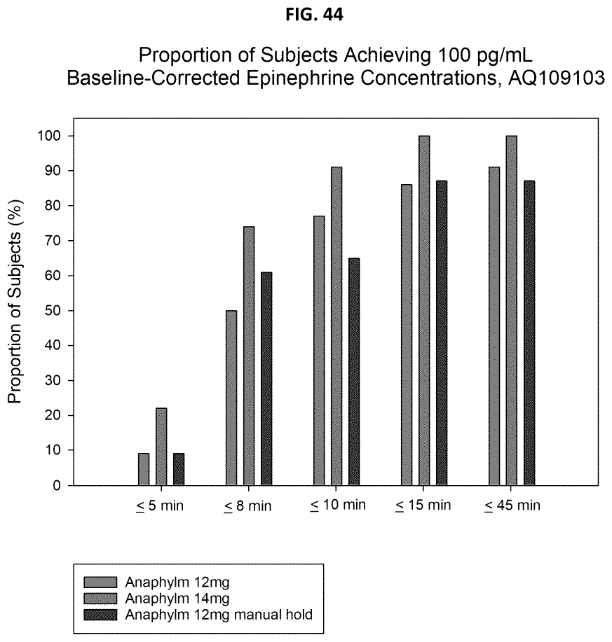

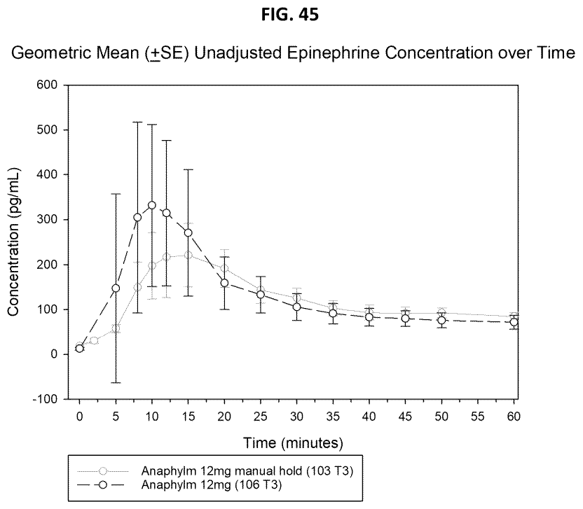

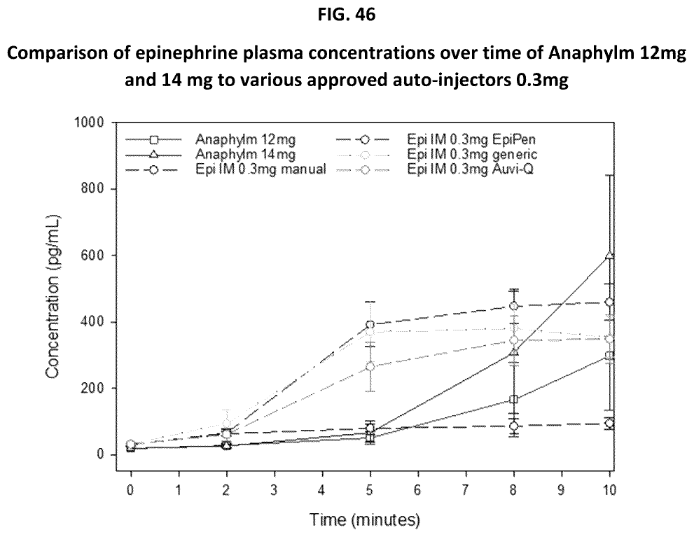

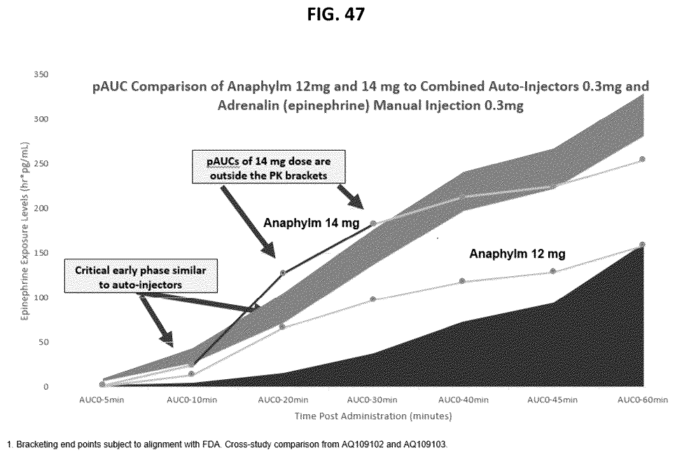

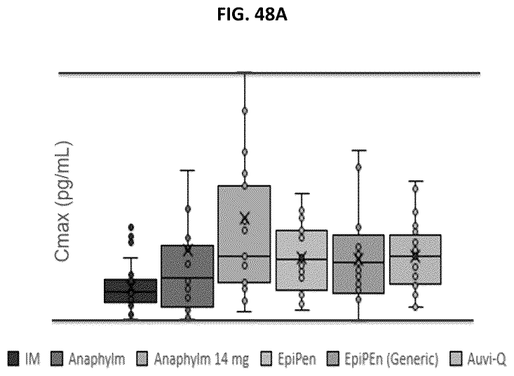

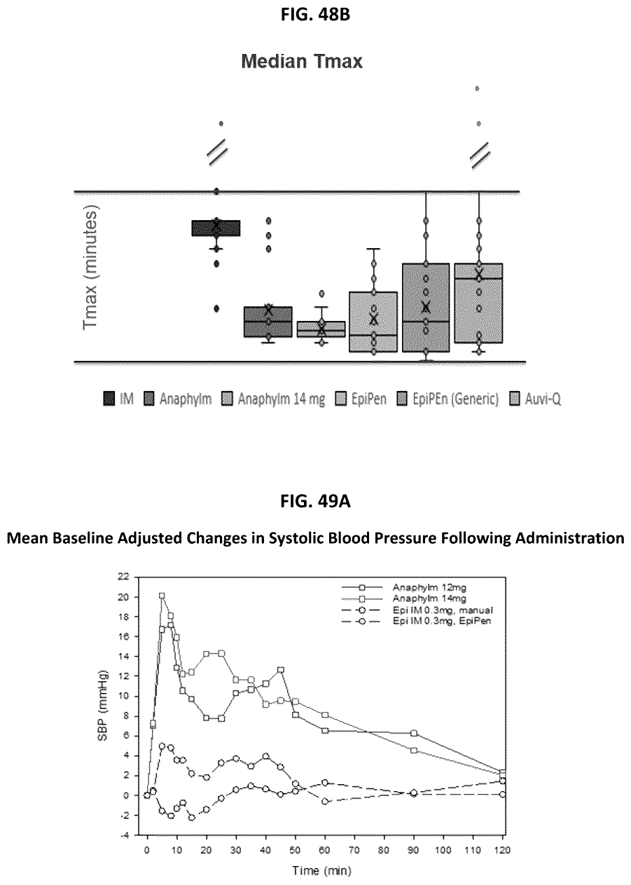

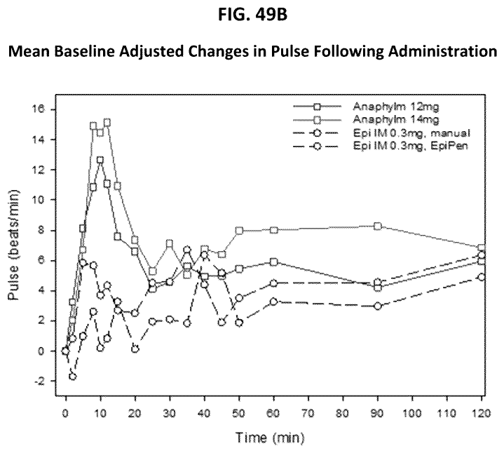

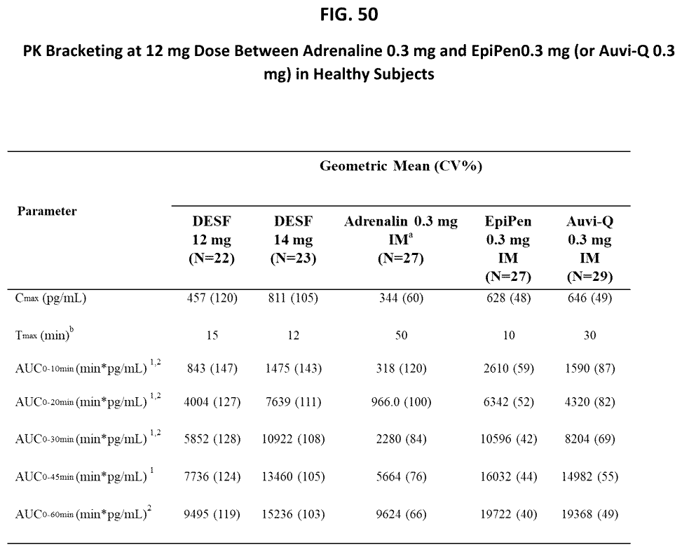

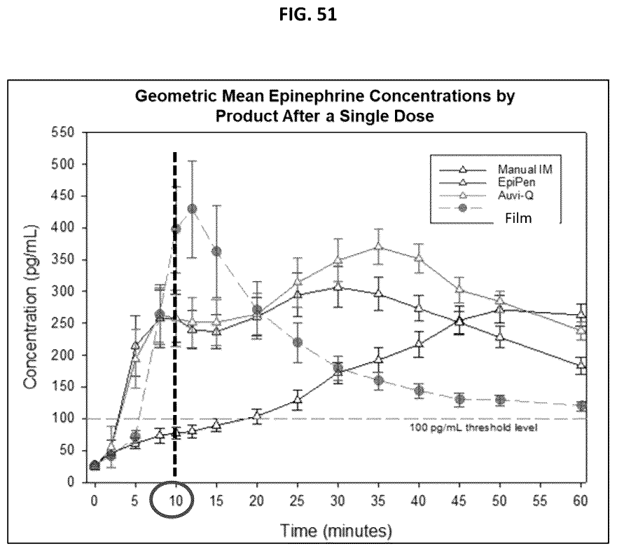

FIG. 51 shows film delivery as a biocomparable to injector delivery. Film achieves rapid PK within first 10 minutes similar to autoinjectors, exceeds Adrenalin beginning at 2 minutes, PK is sustained greater than Adrenalin out to 35 minutes and PK is sustained greater than 100 μg/mL for duration of observation period. Rapid uptake was observed during first 10 minutes.

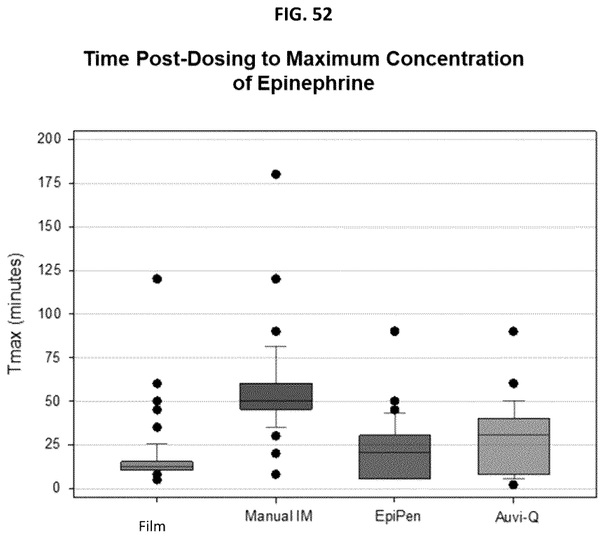

FIG. 52 shows a graph of time to maximum concentration (Tmax) of film significantly more consistent compared to autoinjectors. Tmax is a surrogate for speed of absorption, a critical factor in treating anaphylaxis. Tmax consistency is an important measure of clinical performance. Film Tmax interquartile range (5 min) is significantly more consistent than EpiPen, Auvi Q, and Adrenalin. Film median Tmax of 12 minutes is faster than EpiPen (20 mins), Auvi Q (30 mins), and Adrenalin (50 mins).

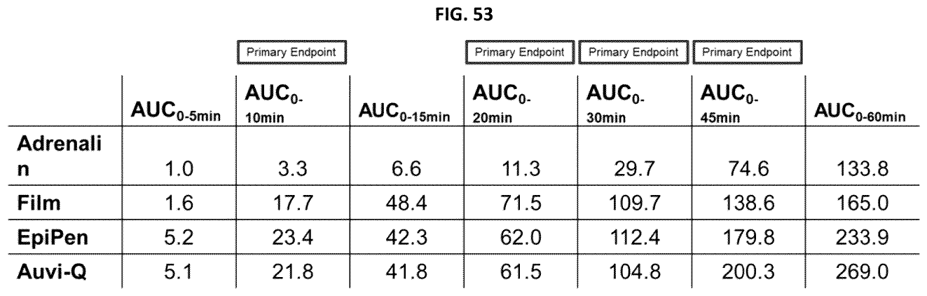

FIG. 53 shows film exposure overtime (pAUC) is consistent and bracketed between injectors through 60 minutes. Film pAUC is substantially the same as EpiPen at AUC₀₋₃₀ₘᵢₙ and is higher than EpiPen at 15 and 20 minutes. Film higher than Adrenalin at all pAUC measurement points. Film is within 80%-120% comparison range to autoinjectors across a majority of critical timepoints. All pAUC measures were predefined as primary, secondary, or exploratory.

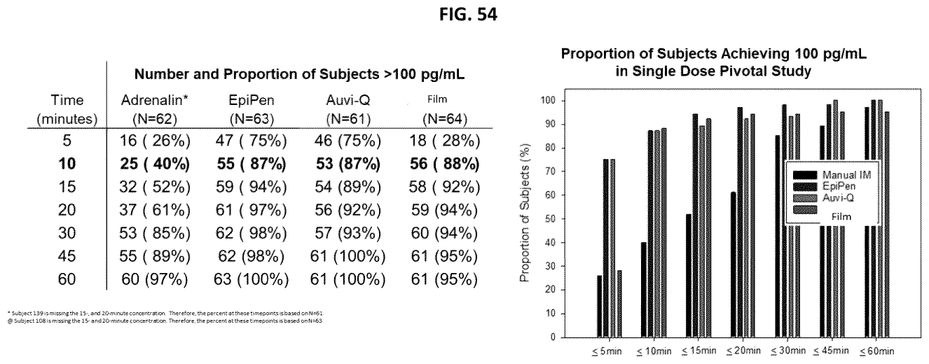

FIG. 54 shows film reaches 100 μg/mL threshold as rapidly as EpiPen and faster than Adrenalin.

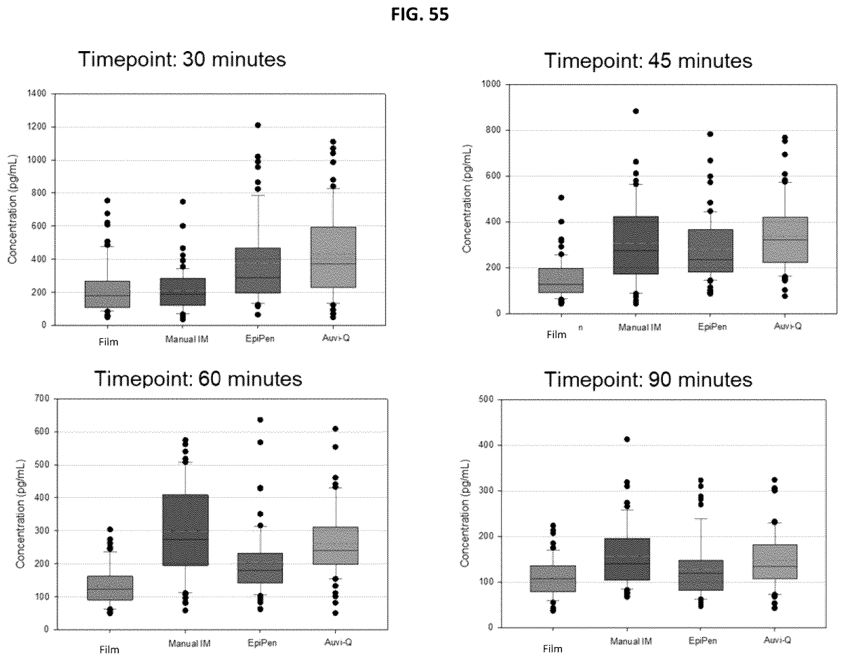

FIG. 55 shows film with comparators at later timepoints (PK Sustainability). Film remains above the 100 μg/mL threshold well past the critical time for treatment. Overlapping exposures with comparators reinforces comparable sustainability beyond the critical time for treatment.

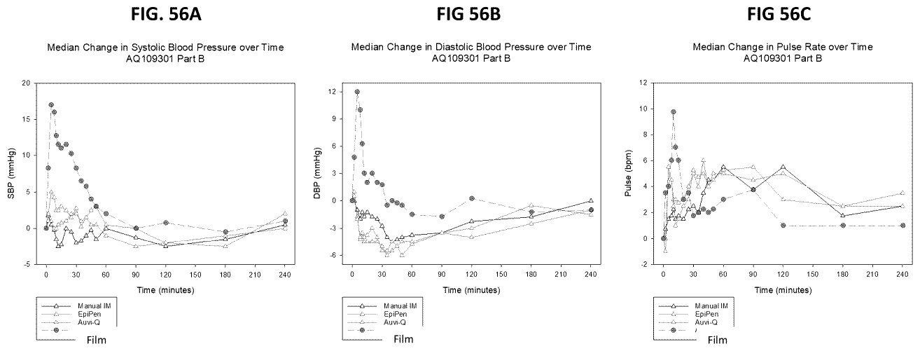

FIG. 56A-56C shows blood pressure over time. Film displays consistent pharmacodynamics with rapid, self-limiting increases in key markers to offset hypotension during anaphylaxis after single dose administration.

A repeat dose study was performed in which primary endpoints predefined as film values greater than or equal to (1) geometric mean concentration at 45 minutes and 60 minutes and (2) AUC₀₋₁₀ₘᵢₙ, AUC₀₋₂₀ₘᵢₙ, and AUC₀₋₃₀ₘᵢₙ.

Geometric Mean Concentrations (pg/mL)

| | 45 | 60 | | |
|---|---|---|---|---|
| | minutes | minutes | Cmax | CV % |
| Anaphylm | 380 | 275 | 2028 | 91% |
| EpiPen | 497 | 347 | 899 | 47% |
| Adrenalin | 365 | 432 | 539 | 56% |

Partial AUCs Bracketing (hr*pg/mL)

| | AUC_{0-10 min} | AUC_{0-20 min} | AUC_{0-30 min} |
|---|---|---|---|
| Adrenalin | 4.1 | 13.8 | 39.0 |
| Anaphylm | 20.3 | 120.8 | 378.5 |
| EpiPen | 21.0 | 69.8 | 172.3 |

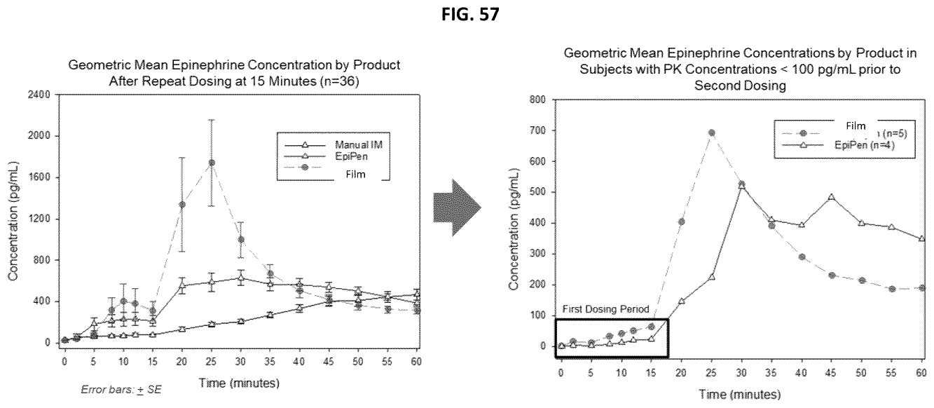

FIG. 57 shows concentration with time with a repeat dose at 15 minutes. Repeat dose study indicates subjects at risk for needing a second dose rapidly exceed threshold value of 100 μg/mL with film. 100% of subjects were >100 μg/mL within 5 minutes of receiving a second dose of film compared to 30 minutes for EpiPen. Overall study data demonstrates repeat dosing is well-tolerated for all evaluated subjects.

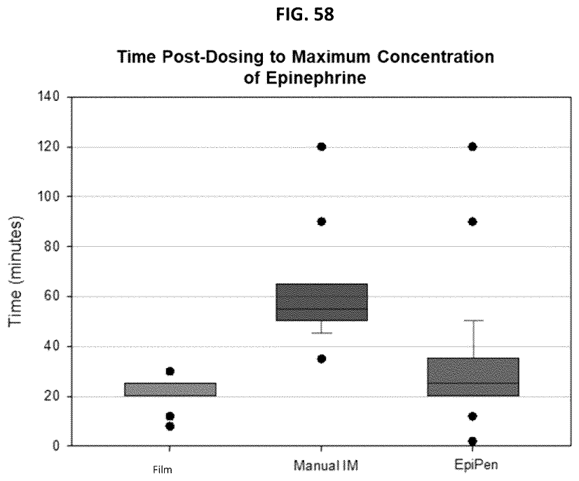

FIG. 58 shows a graph of time post-dosing to maximum concentration of epinephrine. Film median Tmax of 10 minutes after second dosing demonstrates rapid absorption, a critical factor for initial treatment failures. Film Tmax interquartile range (5 min) is significantly more consistent than EpiPen and Adrenalin. Film median Tmax is the same as EpiPen and is significantly faster than Adrenalin.

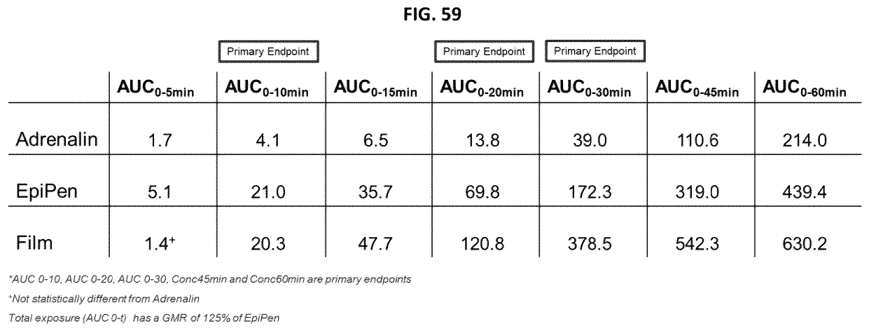

FIG. 59 shows data for exposure over time. Film expo sure over time (pAUC) after repeat dosing meets endpoint (defined as exceeding the lowest injection levels). Repeat dosing given to all subjects at 15 minutes regardless of exposure levels following first dose. Total exposure (AUC₀₋ₜ consistent with EpiPen).

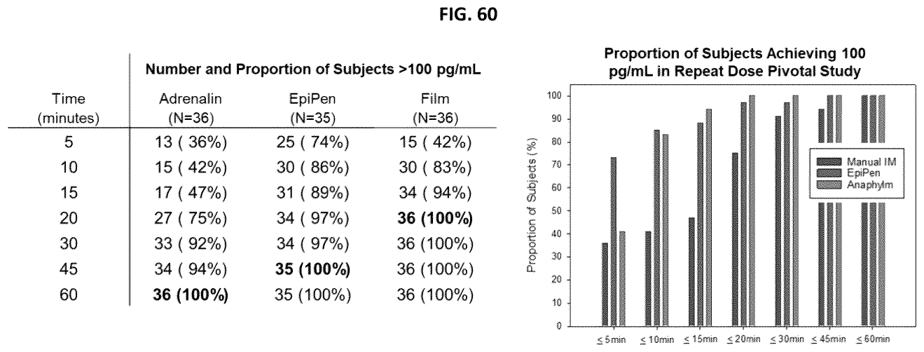

FIG. 60 shows film subjects reach or exceed 100 μg/mL five minutes after second dosing. This is a key threshold level is an important target to achieve hemodynamic stability and curb the progression of anaphylaxis.

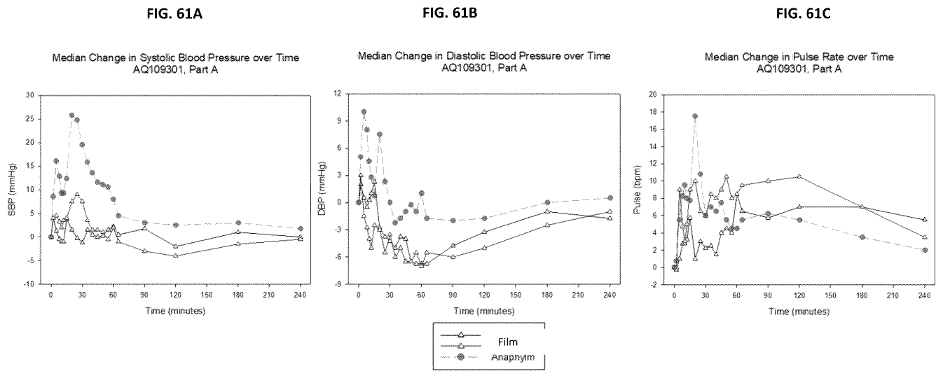

FIG. 61A-C shows graphs of blood pressure over time. FIG. 61A shows median changes in systolic blood pressure. FIG. 61B shows median change in diastolic blood pressure. FIG. 61C shows median changes in pulse.

Film displays consistent pharmacodynamics with rapid, self-limiting increases in key markers to offset hypotension during anaphylaxis after both initial and repeat dose.

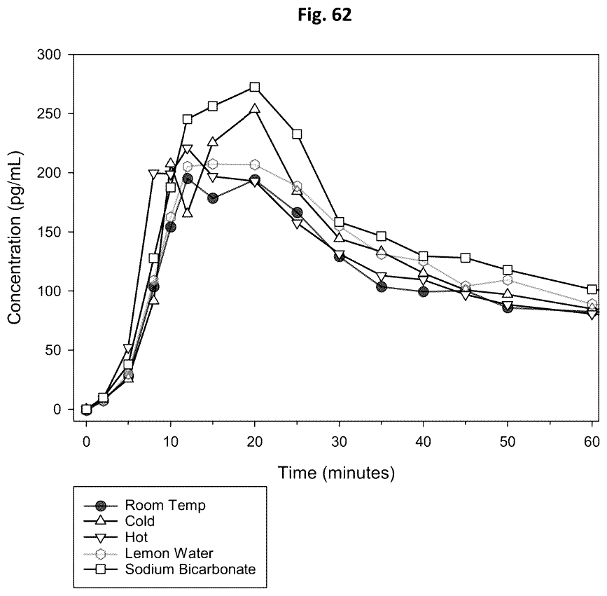

FIG. 62 shows means baseline corrected epinephrine concentration over time by treatment with the film under different pH and temperatures conditions. The study shows the impact of various liquids consumed immediately prior to film administration. The filled circle data points indicate room temperature. The upward pointing triangle data points indicate cold. The downward pointing triangles data points indicate hot. The hexagon data points indicate lemon water. The squares indicate sodium bicarbonate. The Tmax captured was 25 minutes for room temperature water and 20 minutes for all other liquids.

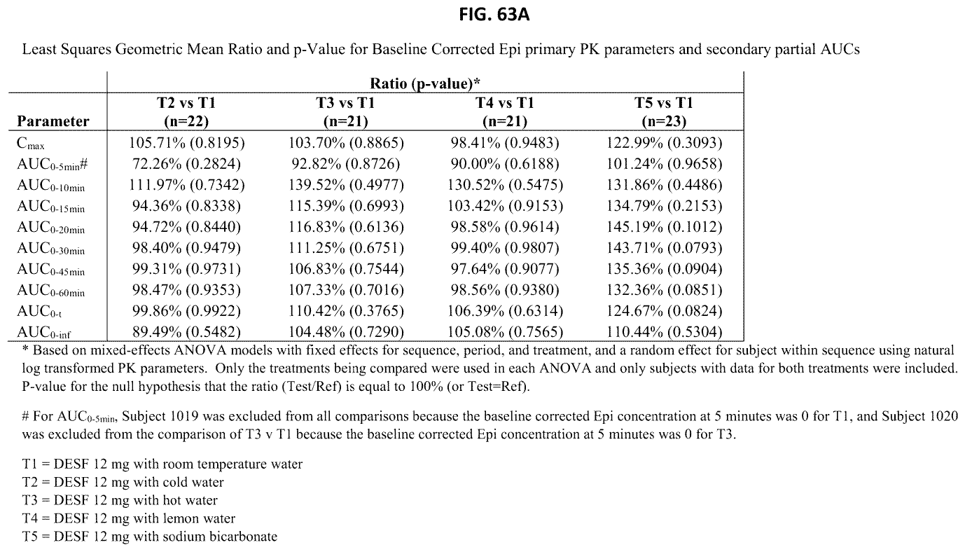

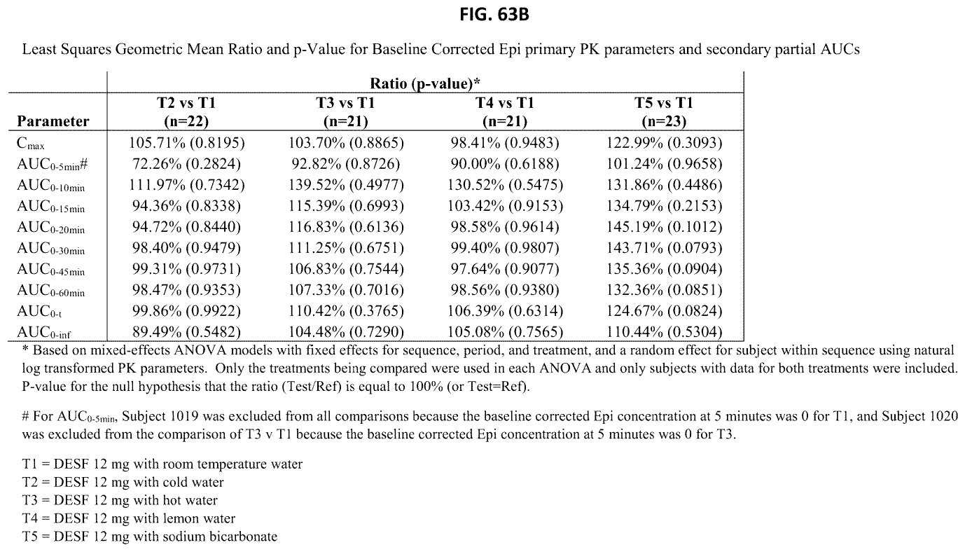

FIG. 63A shows the least squares geometric mean ratio and p-value for baseline corrected epinephrine primary PK parameters and secondary partial AUCs.

FIG. 63B shows the least squares geometric mean ratio and p-value for baseline corrected epinephrine primary PK parameters and secondary partial AUCs.

All references cited herein are hereby incorporated by reference herein in their entirety.

Other embodiments are within the scope of the following claims.

What is claimed is:

1. A method of administering a pharmaceutical composition, comprising:
providing an oral film comprising:
a polymeric matrix;
a pharmaceutically active component including epinephrine or its prodrug in the polymeric matrix; and
an adrenergic receptor interacter;
positioning the film in an oral mucosa for a residence time; and
allowing the film to deliver the pharmaceutically active component.

2. The method according to claim 1, wherein the residence time is no less than 4 minutes.

3. The method according to claim 1, wherein the residence time is no less than 2 minutes.

4. The method according to claim 1, wherein the residence time is 0 minutes.

5. The method according to claim 1, wherein the AUC for 0-10 minutes is equal to or greater than an intramuscular injection of a composition having a same pharmaceutically active component.

6. The method according to claim 1, wherein the AUC for 0-10 minutes is greater than 10 hr*pg/mL for a 4-minutes residence time.

7. The method according to claim 1, wherein the AUC for 0-10 minutes is about 9 hr*pg/mL for a 2-minute residence time.

8. The method according to claim 1, wherein the AUC for 0-10 minutes is about 9 hr*pg/mL for a 0-minute residence time.

9. The method according to claim 1, wherein the AUC for 0-20 minutes is equal to or greater than an intramuscular injection of a composition having a same pharmaceutically active component.

10. The method according to claim 1, wherein the AUC for 0-20 minutes is about 50 hr*pg/mL for a 4-minute residence time.

11. The method according to claim 1, wherein the AUC for 0-20 minutes is about 45 hr*pg/mL for a 2-minute residence time.

12. The method according to claim 1, wherein the AUC for 0-20 minutes is about 30 hr*pg/mL for a 0-minute residence time.

13. The method according to claim 1 wherein the AUC for 0-30 minutes is equal to or greater than an intramuscular injection of a composition having a same pharmaceutically active component.

14. The method according to claim 1, wherein the AUC for 0-30 minutes is about 80 hr*pg/mL for a 4-minute residence time.

15. The method according to claim 1, wherein the AUC for 0-30 minutes is about 50 hr*pg/mL for a 2-minute residence time.

16. The method according to claim 1, wherein the AUC for 0-30 minutes is about 50 hr*pg/mL for a 0-minute residence time.

17. The method according to claim 1, wherein the composition is a film further comprising a polymeric matrix, the pharmaceutically active component being contained in the polymeric matrix.

18. The method according to claim 1, wherein the composition further includes a mixture of two adrenergic receptor interacters.

19. The method according to claim 1, wherein the composition further includes a mixture of three adrenergic receptor interacters.

20. The method according to claim 1, wherein the adrenergic receptor interacter includes an aromatic compound.

21. The method according to claim 1, wherein the adrenergic receptor interacter includes a phenylpropanoid.

22. The method according to claim 1, wherein the adrenergic receptor interacter includes farnesol or Labrasol.

23. The method according to claim 1, wherein the adrenergic receptor interacter includes linoleic acid.

24. The method according to claim 21, wherein the phenylpropanoid is eugenol or eugenol acetate.

25. The method according to claim 21, wherein the phenylpropanoid is a cinnamic acid, cinnamic acid ester, cinnamic aldehyde or hydrocinnamic acid.

26. The method according to claim 21, wherein the phenylpropanoid is chavicol.

27. The method according to claim 21, wherein the phenylpropanoid is safrole.

28. The method according to claim 1, wherein the adrenergic receptor interacter is a phytoextract.

29. The method according to claim 28, wherein the phytoextract is synthetic or biosynthetic.

30. The method according to claim 28, wherein the phytoextract further includes an essential oil extract of a clove plant.

31. The method according to claim 28, wherein the phytoextract further includes 40-95% eugenol.

32. The method according to claim 1, wherein the adrenergic receptor interacter includes a terpenoid, terpene or a sesquiterpene.

33. The method according to claim 1, wherein the oral film comprises an absorbant component.

34. The method according to claim 1, wherein the oral film comprises an adsorbant component.

35. The method according to claim 33, wherein the oral film comprises an amorphous silicon dioxide.

36. The method according to claim 33, wherein the absorbant component is 1-15% by weight of the pharmaceutical composition.

37. The method according to claim 33, wherein the absorbant component has an average surface area of 25-300 m²/g.

38. The method according to claim 33, wherein the absorbant component has an average particle size of 1-10 microns.

39. The method according to claim 33, wherein one or more oil components of the composition can be adsorbed on the absorbant component.

40. The method according to claim 33, wherein can adsorb up to about 0.5-2.0 mL of liquid per gram of absorbant component.

41. The method according to claim 1, wherein the polymer matrix includes a polymer.

42. The method according to claim 41, wherein the polymer is a water soluble polymer.

43. The method according to claim 41, wherein the polymer includes a polyethylene oxide.

44. The method according to claim 42, wherein the polymer includes a cellulosic polymer is selected from the group of: hydroxypropylmethyl cellulose, hydroxyethyl cellulose, hydroxyethylmethyl cellulose, hydroxypropyl cellulose, methylcellulose and carboxymethyl cellulose.

45. The method according to claim 43, wherein the polymeric matrix comprises a cellulosic polymer, polyethylene oxide and polyvinyl pyrrolidone, polyethylene oxide and a polysaccharide, polyethylene oxide, hydroxypropyl methylcellulose and a polysaccharide, or polyethylene oxide, hydroxypropyl methylcellulose, polysaccharide and polyvinylpyrrolidone.

46. The method according to claim 43, wherein the polymeric matrix comprises at least one polymer selected from the group of: pullulan, polyvinyl pyrrolidone, polyvinyl alcohol, sodium alginate, polyethylene glycol, xanthan gum, tragancanth gum, guar gum, acacia gum, arabic gum, polyacrylic acid, methylmethacrylate copolymer, carboxyvinyl copolymers, starch, gelatin, ethylene oxide-propylene oxide co-polymers, collagen, albumin, poly-amino acids, polyphosphazenes, polysaccharides, chitin, chitosan, and derivatives thereof.

47. The method according to claim 1, further comprising a stabilizer.

48. The method according to claim 1, wherein the polymeric matrix comprises a dendritic polymer or a hyperbranched polymer.

49. The method according to claim 1, wherein the prodrug is dipifevrin.

50. The method according to claim 1, wherein the Tmax is 25 minutes or faster.

51. The method according to claim 1, wherein the Tmax is 20 minutes or faster.

52. The method according to claim 1, wherein the Tmax is 15 minutes or faster.

53. The method according to claim 1, wherein swallowing is permitted after the residence time.

54. The method according to claim 1, wherein the pharmaceutically active component includes a prodrug of epinephrine.

55. The method according to claim 54, wherein the prodrug of epinephrine has a half-life of less than one minute.

56. The method according to claim 1, wherein the adrenergic receptor interactor is an active drug moiety, or a derivative or prodrug thereof.

57. A method of administering epinephrine to a subject, comprising:
administering a swallowable composition including a prodrug of epinephrine to the oral cavity of the subject to deliver epinephrine through a digestive tract of the subject.

58. The method according to claim 57, wherein the composition is an oral film.

59. The method according to claim 57, wherein the composition further includes an adrenergic receptor interacter.

60. The method according to claim 57, wherein the composition is formulated to be absorbed through the stomach, intestine or gastrointestinal tract.

61. The method according to claim 57, wherein the composition is administered without food or drink.

62. The method according to claim 57, wherein the composition is swallowed after a residence time.

63. The method according to claim 57, wherein the composition is swallowed without a residence time.

64. The method according to claim 62, wherein the residence time is no less than 4 minutes.

65. The method according to claim 62, wherein the residence time is no less than 2 minutes.

66. The method according to claim 62, wherein the residence time is less than 2 minutes.

67. The method according to claim 62, wherein the AUC for 0-10 minutes is greater than 10 hr*pg/mL for a 4-minutes residence time.

68. The method according to claim 60, wherein the AUC for 0-10 minutes is about 15 hr*pg/mL after food intake.

69. The method according to claim 63, wherein the AUC for 0-10 minutes is about 2 hr*pg/mL with no residence time.

70. The method according to claim 62, wherein the AUC for 0-20 minutes is about 50 hr*pg/mL for a 4-minute residence time.

71. The method according to claim 61, wherein the AUC for 0-20 minutes is about 49 hr*pg/mL after food intake.

72. The method according to claim 63, wherein the AUC for 0-20 minutes is about 28 hr*pg/mL with no residence time.

73. The method according to claim 62, wherein the AUC for 0-30 minutes is about 79 hr*pg/mL for a 4-minute residence time.

74. The method according to claim 61, wherein the AUC for 0-30 minutes is about 65 hr*pg/mL after food intake.

75. The method according to claim 63, wherein the AUC for 0-30 minutes is about 73 hr*pg/mL with no residence time.
